(19) United States
(12) Patent Application Publication
(10) Pub. No.: US 2026/0226011 A1
(43) Pub. Date: Aug. 6, 2026

(54) COMPOSITIONS AND METHODS FOR TREATING OCULAR DISEASES OR DISORDERS

(71) Applicant: Translatum Medicus Inc., Toronto (CA)

(72) Inventors: Shelley Romayne BOYD, Toronto (CA); Natalie PANKOVA, London (GB)

(21) Appl. No.: 19/155,073

(22) PCT Filed: Feb. 9, 2024
(86) PCT No.: PCT/IB2024/000081

§ 371 Date: Aug. 8, 2025

Related U.S. Application Data
(60) Provisional application No. 63/484,395, filed on Feb. 10, 2023.
(60) Provisional application No. 63/539,413, filed on Sep. 20, 2023.
(60) Provisional application No. 63/592,789, filed on Oct. 24, 2023.

(51) Int. Cl. C07D 231/56; A61K 31/05; A61K 31/137; A61K 31/192; A61K 31/405; A61K 31/416; A61K 31/436; A61K 31/498; A61K 31/57; A61K 31/65; A61K 47/54; A61K 47/55; A61K 47/60; A61K 47/64; A61K 47/68; A61P 27/02; C07D 403/12

(52) U.S. Cl. CPC C07D 231/56; A61K 31/05; A61K 31/137; A61K 31/192; A61K 31/405; A61K 31/416; A61K 31/436; A61K 31/498; A61K 31/57; A61K 31/65; A61K 47/54; A61K 47/55; A61K 47/60; A61K 47/64; A61K 47/6803; A61P 27/02; C07D 403/12

(57) ABSTRACT

Disclosed herein are methods, compounds, and compositions that are useful for the treatment or prevention of an ocular disease or disorder.

### CROSS-REFERENCE TO RELATED APPLICATIONS

This application claims the benefit of priority to U.S. Provisional Patent Application Nos. 63/484,395, filed Feb. 10, 2023, 63/539,413, filed Sep. 20, 2023, and 63/592,798, filed Oct. 24, 2023, each of which is incorporated by reference herein in its entirety.

### FIELD

This disclosure relates to, in part, methods and compositions that are useful for the treatment or prevention of an ocular disease or disorder, including the discovery of agents, including immunomodulatory agents, that are efficacious against these disorders and which have physiologically relevant release profiles.

### BACKGROUND

Ocular diseases or disorders, which may reduce or eliminate ones sight, among other effects, are a major medical concern. For instance, age-related macular degeneration (AMD) is a leading cause of ocular dysfunction, including irreversible blindness, especially in patients over 50 years old. All patients start with early so-called “dry” AMD. This is characterized by deposits that can be seen clinically in the posterior pole of the eye known as the macula. Advanced AMD can take two forms-late “wet” or late “dry”. The primary pharmaceutical option in advanced “wet” AMD is regular intra-ocular injections of antiangiogenic drugs. These injections are given after pathological new blood vessels grow beneath or into the central retina, where they leak, bleed or cause tissue damage and scarring. By contrast, there are no treatments for late or early dry AMD. Late dry AMD is characterized by the death or atrophy of the photoreceptors and their nurse cells, the retinal pigment epithelium (RPE). Patches of tissue loss are known as “geographic atrophy” GA.

A major challenge for the treatment of ocular diseases or disorders is delivery. Delivery of drugs to the targeted ocular tissues is confounded by various ocular barriers, such as precorneal, dynamic and static ocular barriers. Also, even when targeting is possible, therapeutic drug levels are not maintained for longer duration in ocular target tissues. Accordingly, even the most successful ocular therapeutics, e.g. anti-VEGF therapies for wet AMD, require regular and invasive administration that is expensive and complicated by possible lack of patient adherence.

Accordingly, there is presently a paucity of satisfactory agents for effective treatment of ocular diseases or disorders. Therefore, there remains a need for therapies that are useful for treating these disorders. Further, there is a need for effective delivery of these agents to treat these disorders in manner that is practical.

### SUMMARY OF THE DISCLOSURE

Accordingly, in one aspect, the disclosure provides compounds that are amenable to ocular delivery, e.g. sustained release in the eye. In one aspect, the disclosure provides a compound of formula (I), or a pharmaceutically acceptable salt thereof:

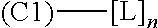

wherein in formula (I):
C1 is bindarit or an analogue thereof;
L is independently at each occurrence a linker covalently linked to C1, wherein each L is optionally further covalently linked to an active agent C2, and each occurrence of C2 may be the same or different; and
n is an integer selected from 1 to 4.

In some embodiments, C1 comprises a group selected from:

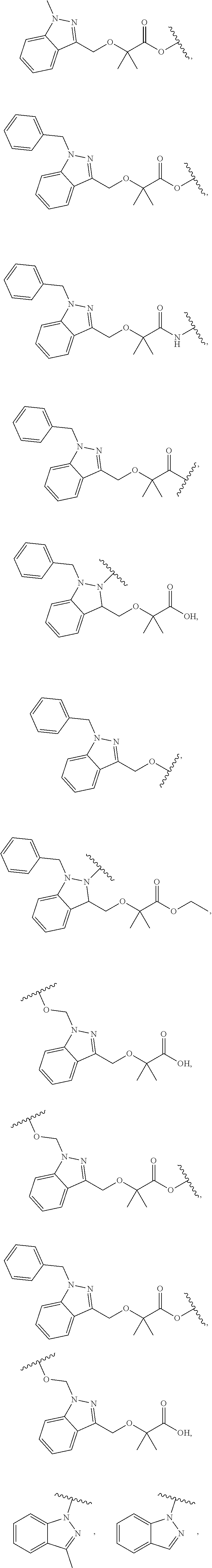

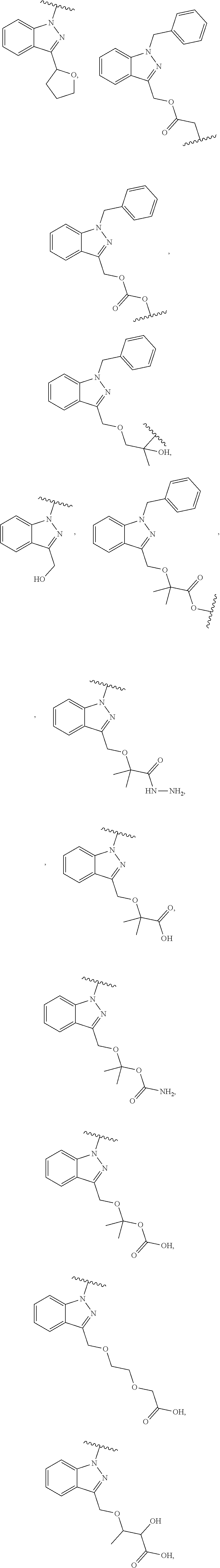

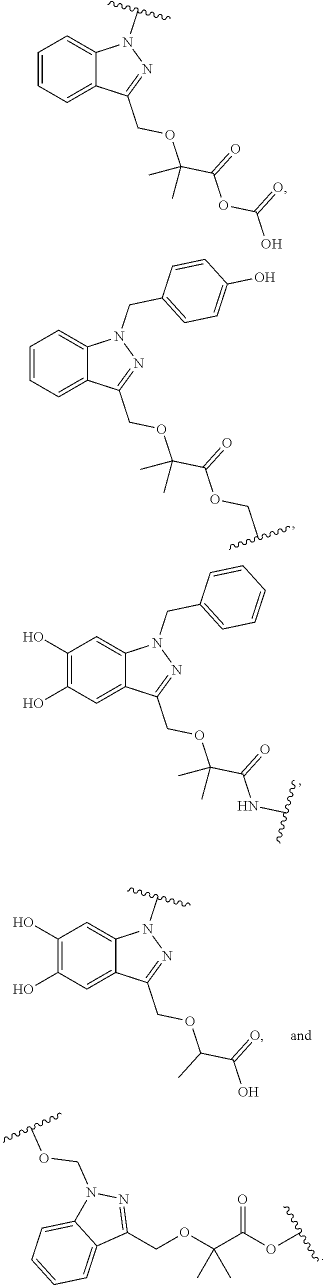

In some embodiments, the compound of formula (I) is a compound of formula (Ia), or a pharmaceutically acceptable salt thereof. In some embodiments, the compound of formula (I) is a compound of formula (Ib), or a pharmaceutically acceptable salt thereof. In some embodiments, the compound of formula (I) is a compound of formula (Ic), or a pharmaceutically acceptable salt thereof. In some embodiments, the compound of formula (I) is a compound of formula (Id), or a pharmaceutically acceptable salt thereof. In some embodiments, the compound of formula (I) is a compound of formula (Ie), or a pharmaceutically acceptable salt thereof. In some embodiments, the compound of formula (I) is a compound of formula (Ia1), or a pharmaceutically acceptable salt thereof. In some embodiments, the compound of formula (I) is a compound of formula (Ia2), or a pharmaceutically acceptable salt thereof. In some embodiments, the compound of formula (I) is a compound of formula (Ia3), or a pharmaceutically acceptable salt thereof. In some embodiments, the compound of formula (I) is a compound of formula (Iia), or a pharmaceutically acceptable salt thereof. In some embodiments, the compound of formula (I) is a compound of formula (i2a), or a pharmaceutically acceptable salt thereof. In some embodiments, the compound of formula (I) is a compound of formula (i2b), or a pharmaceutically acceptable salt thereof. In some embodiments, C1 is bindarit ethyl ester or an analogue thereof. In some embodiments, the compound of formula (I) is a compound of formula (Iib), or a pharmaceutically acceptable salt thereof. In some embodiments, the compound of formula (I) is a compound of formula (Iic) or a pharmaceutically acceptable salt thereof. In some embodiments, the compound of formula (I) is a compound of formula (IIIB), or a pharmaceutically acceptable salt thereof. In some embodiments, the compound of formula (I) is a compound of formula (IV), or a pharmaceutically acceptable salt thereof. In some embodiments, the compound of formula (I) is a compound of formula (V), or a pharmaceutically acceptable salt thereof.

In some embodiments, wherein in formula (Iia), (Iic), (i2a), or (i2b), at least one of (a)-(e) is satisfied: (a) R^{1a}, R^{1b}, R^{1c}, R^{1d}, and Rie are each hydrogen; (b) R² is hydrogen; (c) R^{3a}, R^{3b}, R^{3c}, and R^{3d} are each hydrogen; (d) R⁴ is hydrogen; or (e) R^{5a} and R^{5b} are each hydrogen.

In some embodiments, each L independently comprises one or more of a bond, —(CH₂CH₂O)—, —C(O), —(CH₂)—, —O—, —C(O)O—, —OC(O)—, —C(O)NR^{a}—, —NR^{a}C(O)—, —S—, —S—S—, —SCH₂CH₂S—, —NR^{a}—, —S(O)—, —S(O)₂—, —CR^{a}₂—, —C(O)S—, —SC(O)—, —C(O)NR^{a}SO₂—, —SO₂NR^{a}C, (O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

In some embodiments, each L independently comprises one or more of a PEG-based linker, a polyester, polypropylene, polyolefin, polyurethane, Alhydrogel, vitamin K, caffeine, menadione, benzoic acid, cholesterol, PVP, β-cyclodextrin,

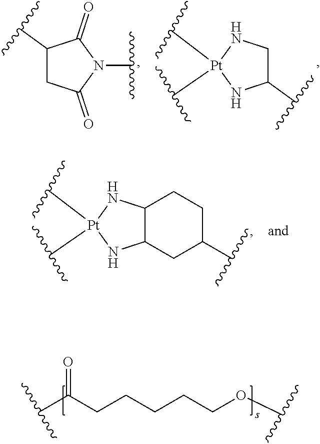

wherein s is 1 to 5:

In some embodiments, the PEG-based linker comprises one or more of an amine reactive PEG-based linker (e.g. Bis-PEG-acid, Bis-PEG-NHS, Boc-PEG, Fmoc-PEG, PEG Acid, PEG Aldehyde, PEG NHS ester, PEG Phosphonate, PEG PFP ester, PEG Tosylate), a biotinylated PEG-based linker (e.g. PEG-biotin), a branched PEGbased linker; a reactive carbonyl PEG-based linker (e.g. aminooxy-PEG), a carboxyl and/or active ester reactive PEG-based linker (e.g. amine PEG), a click-reagent PEG-based linker (e.g. alkyne PEG, azide-PEG); a copper-free click chemistry PEG-based linker (e.g. DBCO-PEG, BCN-PEG); a Cu-catalyzed click chemistry PEG-based linker (e.g. propargyl-PEG); a fluorescent PEG labeling and homobifunctional PEG-based linker (e.g. bis-PEG-acid, bis-PEG-NHS, bis-PEG-PFP, bis-propargyl-PEG, amine-PEG-amine, azido-PEG-azide, bromo-PEG-bromide), a lipid PEG, a metal surface binding and thiol reactive PEG-based linker (e.g. Thiol PEG, Bromo-PEG, Mal PEG), PEG-Pasp, PEG-Pglu, PEG-b-PMPMC, PEG-PGLA,

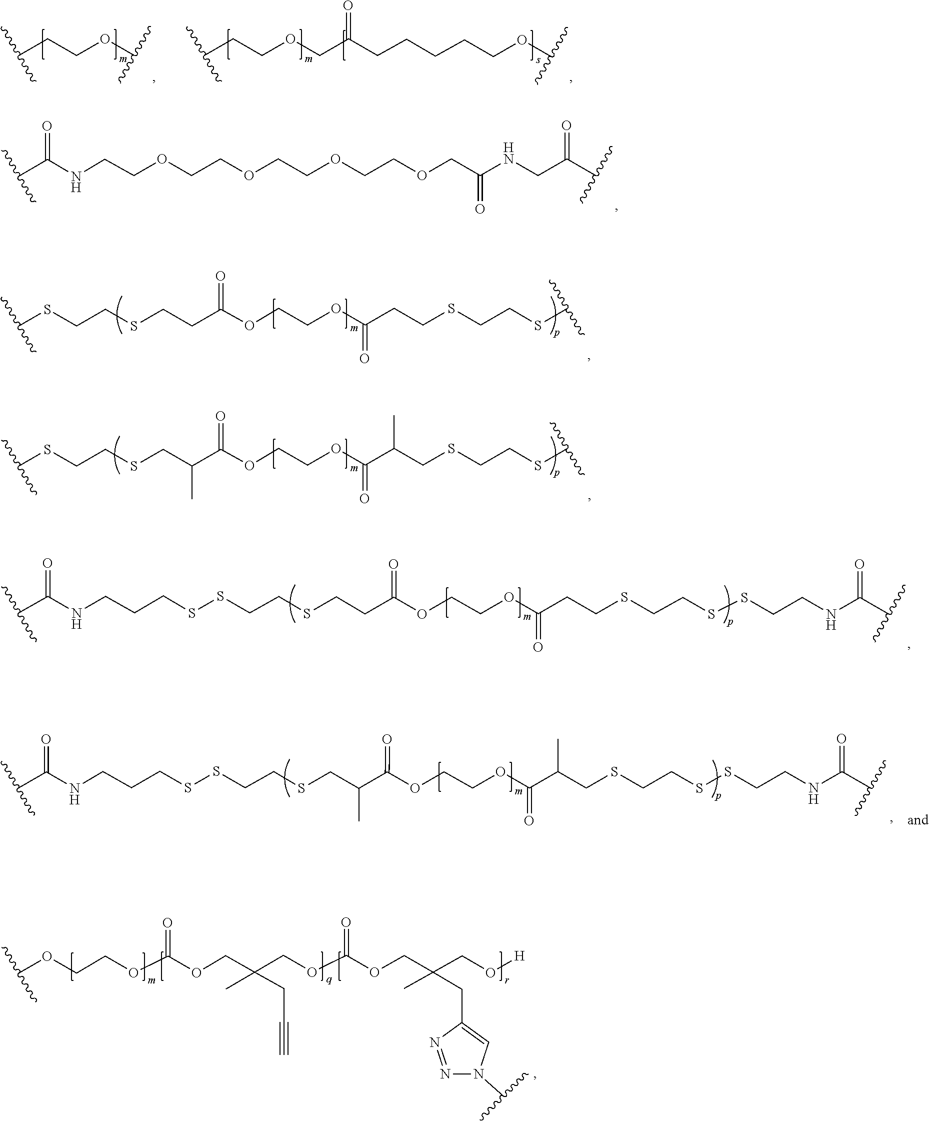

wherein:
m is an integer selected from 1 to 5;
p is an integer selected from 1 to 3;
q is an integer selected from 1 to 5;
r is an integer selected from 1 to 5; and
s is an integer selected from 1 to 5.

In some embodiments, one or more L is further covalently linked to C2. In some embodiments, C2 comprises one or more of an anti-vascular endothelial growth factor (VEGF) agent, a modulator of the complement cascade, an angiotensin-converting enzyme (ACE) inhibitor, a peroxisome proliferator-activated receptor (PPAR) modulator, a renin inhibitor, a corticosteroid, an antibody, a soluble receptor, a biological molecule, a nuclear receptor protein (e.g. LXR), a Liver X receptor (LXR) agonist, a steroid (e.g. fluocinolone acetonide, dexamethasone), and an agent that modulates autophagy. In some embodiments, the PPAR modulator is selected from a PPAR-α modulator, a PPAR-β/δ modulator, and a PPAR-γ modulator. In some embodiments, C2 comprises one or more of bindarit or an analogue thereof, an anti-angiogenic agent, including but not limited to a biological anti-angiogenic agent, (e.g. angiopoietin and placental growth factor), an anti-VEGF agent, including but not limited to a low molecular weight anti-VEGF agent, a nutraceutical, optionally resveratrol, one or more peptides, optionally taurine, carnosine or aspartame, or one or more proteins, optionally a an anti-VEGF antibody or a soluble receptor. In some embodiments, the anti-angiogenic agent is selected from Axitinib (Inlyta), Bevacizumab (Avastin), Cabozantinib (Cometriq), Everolimus (Afinitor, Zortress), Lenalidomide (Revlimid), Pazopanib (Votrient), Ramucirumab (Cyramza), Regorafenib (Stivarga), Sorafenib (Nexavar), Sunitinib (Sutent), Thalidomide (Synovir, Thalomid), Vandetanib (Caprelsa), Ziv-aflibercept (Zaltrap), Aflibercept (Eylea), Ranibizumab (Lucentis), Brolucizumab, Abicipar (a DARPin), KSI-301 Antibody Biopolymer Conjugate (Kodiak sciences) (e.g. for extended release in ophthalmology), OPT-302 (Opthea) (VEGF-C/D trap molecule), Faricimab (anti-VEGF & anti-angiopoietin-2), DE-122, and anti-endoglin (Tracon & Santen) (e.g. for anti-angiogenesis in ophthalmology). In some embodiments, the nutraceutical agent is selected from nutraceutical agents include, resveratrol, taurine, carnosine, quercetin, curcumin, glycyrrhizin, naringin, folate, glutathione, n-acetylcysteine, alpha lipoic acid, quercetin, zinc, vitamin a, vitamin c, vitamin d, vitamin k2, vitamin b, vitamin b6, vitamin b9, vitamin b12, vitamin e, magnesium, bromelain, cannabidiolic acid, cannabigerolic acid, cannabidiol, cannabigerol, cannabinol, nigeIIa sativa/thymoquinone, selenium, curcumin, piperine, astaxanthin, and sulforaphane. In some embodiments, the peptide is selected from a monopeptide (e.g. taurine, or carnosine) and a dipeptide (e.g. aspartame and glutamine-taurine). In some embodiments, the protein is selected from an anti-VEGF antibody (e.g. Ranibizumab) or a soluble receptor (e.g. solVEGFR1 [sol flt-1] or solVEGFR2). In some embodiments, the low molecular weight anti-VEGF agent is selected from thalidomide (Synovir, Thalomide), an mTOR inhibitor (e.g. temsirolimus, everolimus (Afinitor, Zortress), tacrolimus or rapalogues (eg rapamycin), or corticosteroids (dexamethasone, Fluocinolone acetonide, triamcinolone), aflibercept, and a tyrosine kinase inhibitor. In some embodiments, the tyrosine kinase inhibitor is selected from Vatalanib (PTK787), lapatinib, Sunitinib (Sutent), Sorafenib (Nexavar), Ramucirumab (Cyramza), Nintedanib, Lenvatinib, Anlotinib, Fruquintinib, Apatinib, Pazopanib (Votrient), Axitinib (Inlyta), Cabozantinib (Cometriq), Vandetanib (Caprelsa), and Regorafenib (Stivarga). In some embodiments, wherein the biological molecule is an antibody, an antibody fragment, a soluble receptor (e.g. solVEGFR1 [sol flt-1] or solVEGFR2), a DARPin, a microRNA (miRNA), Bevacizumab (Avastin), Ziv-aflibercept (Zaltrap), Aflibercept (Eylea), Ranibizumab (Lucentis), Brolucizumab (Beovu), Abicipar (AbbVie, a DARPin), KSI-301 Antibody Biopolymer Conjugate (Kodiak sciences) (e.g. for extended release in ophthalmology), OPT-302 (Opthea) (VEGF-C/D trap molecule), Faricimab (anti-VEGF & anti-angiopoietin-2), and DE-122, anti-endoglin antibody. In some embodiments, C2 comprises a polyphenol, optionally wherein the polyphenol is selected from catechin, resveratrol, hesperidin, chlorogenic acid, flavonols, isoflavones, tannins, quercetin, and anthocyanins. In some embodiments, C2 comprises resveratrol.

In some embodiments, bindarit or the analogue thereof comprises a group selected from:

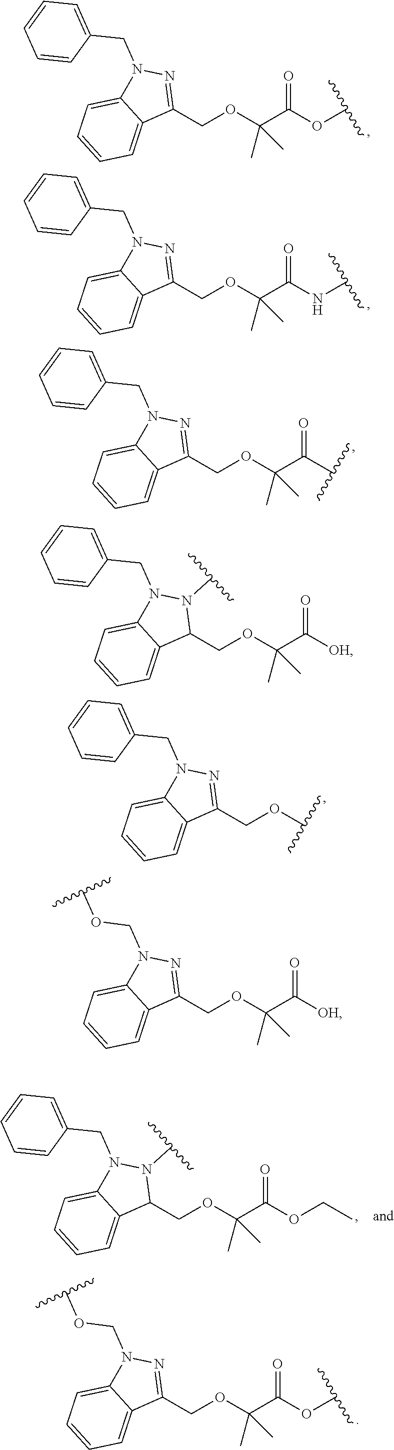

In some embodiments, the compound of formula (I) is a compound of formula (If), or a pharmaceutically acceptable salt thereof. In some embodiments, the compound of formula (I) is a compound of formula (Ig), or a pharmaceutically acceptable salt thereof. In some embodiments, the compound of formula (I) is a compound of formula (Ih), or a pharmaceutically acceptable salt thereof. In some embodiments, the compound of formula (I) is a compound of formula (Ii), or a pharmaceutically acceptable salt thereof. In some embodiments, the compound of formula (I) is a compound of formula (Ij), or a pharmaceutically acceptable salt thereof. In some embodiments, the compound of formula (I) is a compound of formula (Ik), or a pharmaceutically acceptable salt thereof. In some embodiments, the compound of formula (I) is a compound of formula (Im), or a pharmaceutically acceptable salt thereof. In some embodiments, the compound of formula (I) is a compound of formula (In), or a pharmaceutically acceptable salt thereof.

In some embodiments, the compound of formula (I) is a macrocycle. In some embodiments, the compound of formula (I) is a compound of formula (IIIa), or a pharmaceutically acceptable salt thereof. In some embodiments, the compound of formula (I) is a compound of formula (IIIb), or a pharmaceutically acceptable salt thereof. In some embodiments, the compound of formula (I) is a compound of formula (Iic), or a pharmaceutically acceptable salt thereof. In some embodiments, the compound of formula (I) is a compound of formula (IV), or a pharmaceutically acceptable salt thereof. In some embodiments, the compound of formula (I) is a compound of formula (V), or a pharmaceutically acceptable salt thereof.

In some embodiments, the compound of formula (I) is a homodimer. In some embodiments, the compound of formula (I) is a heterodimer.

In some embodiments, the compound of formula (I) is of any one of formulas 1001 to 1005, or a pharmaceutically acceptable salt thereof.

In various aspects, the present disclosure provides a compound of formula (I) wherein:
C1 is selected from bindarit or an analogue thereof and bindarit ethyl ester or an analogue thereof;
L comprises one or more of a bond, —(CH₂CH₂O)—, —C(O), —(CH₂)—, —O—, —C(O)O—, —OC(O)—, —C(O)NR^{a}—, —NR^{a}C(O)—, —S—, —S—S—, —SCH₂CH₂S—, —NR^{a}—, —S(O)—, —S(O)₂—, —CR^{a}₂—, —C(O)S—, —SC(O)—, —C(O)NR^{a}SO₂—, —SO₂NR^{a}C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl:
L is further covalently linked to C2;
C2 is selected from PTK787, aflibercept, Brolucizumab, Triamcinolone, Dexamethasone, OPT-302, and DE-122; and
n is 1.

In some embodiments, L independently one or more of a PEG-based linker, a polyester, polypropylene, polyolefin, polyurethane, Alhydrogel, vitamin K, caffeine, menadione, benzoic acid, cholesterol, PVP, β-cyclodextrin,

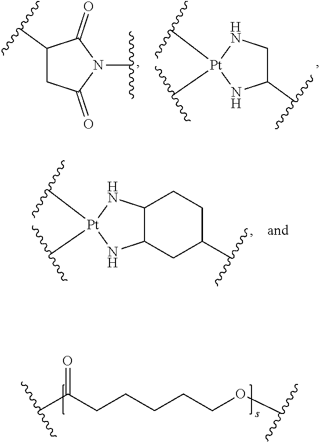

wherein s is 1 to 5, optionally wherein the polyester is selected from: polylactic acids, poly-L-lactic acid (PLLA), and a random copolymer of L-lactic acid and D-lactic acid, and derivatives thereof, polycaprolactone, polyhydroxybutyric acid, polyhydroxyvaleric acid, polyethylene succinate, polybutylene succinate, polybutylene adipate, polymalic acid, polyglycolic acid, polysuccinate, polyoxalate, polybutylene diglycolate, and polydioxanone.

In some embodiments, the PEG-based linker comprises one or more of an amine reactive PEG-based linker (e.g. Bis-PEG-acid, Bis-PEG-NHS, Boc-PEG, Fmoc-PEG, PEG Acid, PEG Aldehyde, PEG NHS ester, PEG Phosphonate, PEG PFP ester, PEG Tosylate), a biotinylated PEG-based linker (e.g. PEG-biotin), a branched PEG-based linker; a reactive carbonyl PEG-based linker (e.g. aminooxy-PEG), a carboxyl and/or active ester reactive PEG-based linker (e.g. amine PEG), a click-reagent PEG-based linker (e.g. alkyne PEG, azide-PEG); a copper-free click chemistry PEG-based linker (e.g. DBCO-PEG, BCN-PEG); a Cu-catalyzed click chemistry PEG-based linker (e.g. propargyl-PEG); a fluorescent PEG labeling and homobifunctional PEG-based linker (e.g. bis-PEG-acid, bis-PEG-NHS, bis-PEG-PFP, bis-propargyl-PEG, amine-PEG-amine, azido-PEG-azide, bromo-PEG-bromide), a lipid PEG, a metal surface binding and thiol reactive PEG-based linker (e.g. Thiol PEG, Bromo-PEG, Mal PEG), PEG-Pasp, PEG-Pglu, PEG-b-PMPMC, PEG-PGLA,

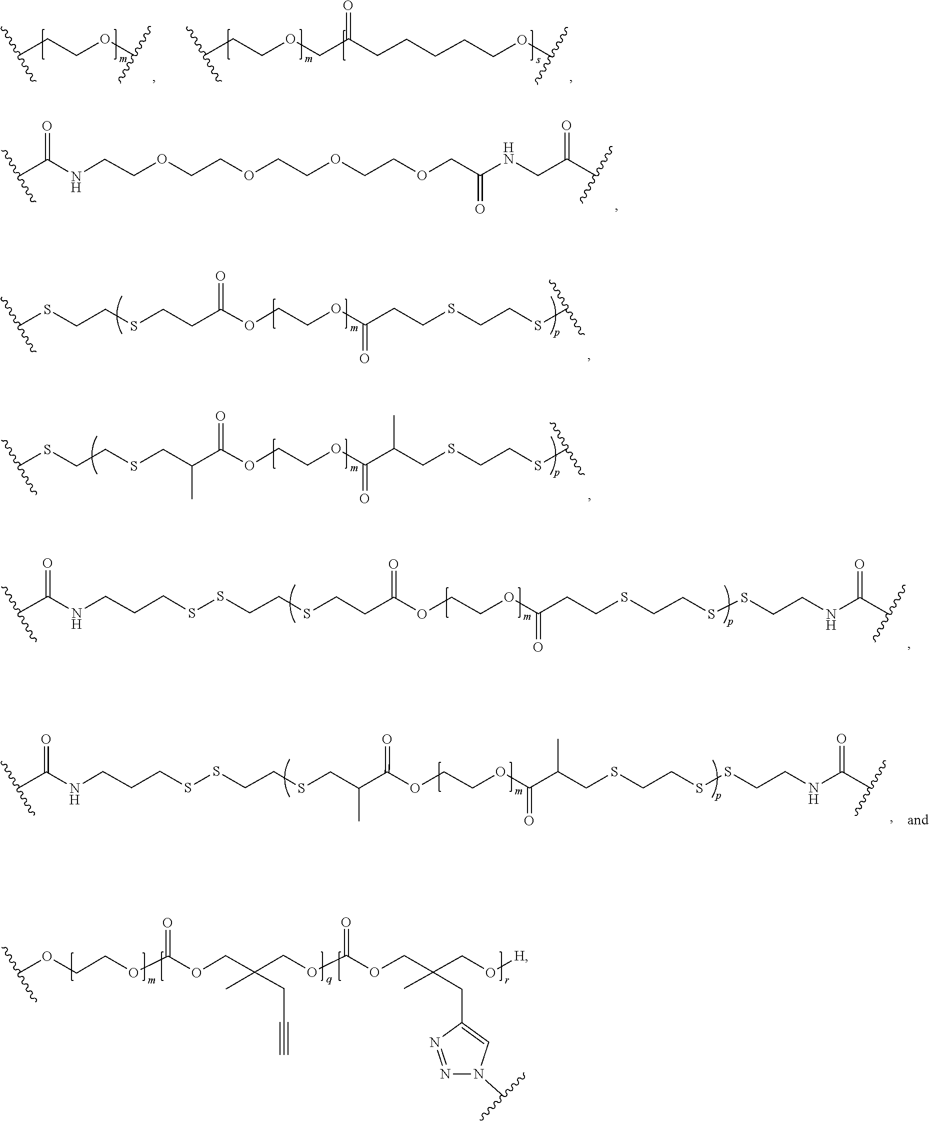

wherein:
m is an integer selected from 1 to 5;
p is an integer selected from 1 to 3;
q is an integer selected from 1 to 5;
r is an integer selected from 1 to 5; and
s is an integer selected from 1 to 5.

In various aspects, the present disclosure relates to a pharmaceutical composition comprising one or more compounds of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIIa), formula (VIIb), and formulas 1001 to 1005, and a pharmaceutically acceptable carrier. In some embodiments, wherein the pharmaceutical composition comprises two or more compounds, wherein at least one compound is a homodimer and at least one compound is a heterodimer. In some embodiments, the molar ratio of the homodimer to the heterodimer is selected from between about 1:99 to about 99:1. In some embodiments, the pharmaceutical composition is formulated for extended release. In some embodiments, the pharmaceutical composition is formulated for ocular delivery.

In various aspects, the present disclosure relates to a method for treating or preventing an ocular disease or disorder, comprising administering to a subject in need thereof a pharmaceutical composition comprising an effective amount of one or more compounds of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, and a pharmaceutically acceptable carrier or excipient. In some embodiments, In some embodiments, the ocular disease or disorder is one or more of dry age-related macular degeneration (AMD), reticular drusenoid disease (RPD), white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), diffuse subretinal fibrosis (DSF)), LORDs, and central serous retinopathy (CSR). In some embodiments, the ocular disease or disorder is one or more of Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; and Wegener granulomatosis. In some embodiments, the method further comprises reducing the subject's expression of one or more of CD64, IDO, SOCS1, CXCL10 Marco, Nos2, 1112b, Ptgs2 (Cox2), MCP-1 (CCL2), MCP-2 (CCL8), MCP-3 (CCL7), 1123a (H23p19), Idol, Adipoq, Ccl20, IL17 (subtype a)Ccl5, CD163, Cx3Cr1, Faslg, Gfap, Csf2, Icaml, Ifng, 1110, 1112b, 1113, 1117, 1118, 1M b, 1122, 114, 116, Klf4, Mrd, Myd88, Nlrp3, Pparv, Tgfb1, Tlr4, Tnf, Vcaml, Ccl2, Ccl5, Ccl7, Ccr2, Socsl, Socs3, Statl, Stat3, and Stat6. In some embodiments, the subject is not undergoing treatment with and/or is unresponsive and/or is substantially unresponsive and/or is partially responsive to one or more of an anti-factor D antibody (e.g. lampalizumab (Genentech)), an anti-β-amyloid (anti-AB) antibody (e.g. GSK933776 (GSK), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), Zimura (Iveric), an anti-C3 complement antibody (e.g. APL-2 (Apellis) or related compounds thereof, NGM621 (NGM Biopharmaceutical), doxycycline (ORACEA), emixustat hydrochloride, rapalogues (e.g. sirolimus (RAPAMUNE)), metformin or analogues thereof, and glatiramer acetate (COPAXONE). In some embodiments, the subject has evidence of intermediate AMD as confirmed by the presence of at least 1 large druse and/or retinal pigment changes and/or geographic atrophy of retinal pigment epithelium (RPE) not in the center of the fovea. In some embodiments, the subject has early stage dry macular degeneration as evidenced by several small drusen and/or a few medium-sized drusen and/or mild hypopigmentation or hyperpigmentation of the retinal pigment epithelium (RPE) in at least one eye. In some embodiments, the subject has late stage dry macular degeneration as evidenced by a large number of medium-sized drusen and/or one or more large drusen and/or degeneration of retinal pigment epithelium (RPE) in the foveal center and/or presence of scars in the foveal area and/or loss of central vision. In some embodiments, the subject has late stage dry macular degeneration as evidenced by several large drusen and/or an extensive breakdown of cells in the macula. In some embodiments, small drusen are less than about 63 jum in diameter and/or pigment. In some embodiments, medium-sized drusen are between about 63 μm and 125 μm in diameter and/or pigment. In some embodiments, large drusen are greater than about 125 μm in diameter and/or pigment. In some embodiments, the subject has reticular pseudodrusen, or early or late stage dry AMD as evidenced by subretinal drusenoid deposits. In some embodiments, the subject has a variant of AMD. In embodiments, the subject has atrophic AMD. In some embodiments, the variant of AMD is selected from polypoidal choroidal vasculopathy (PCV) and retinal angiomatous proliferation (RAP). In some embodiments, subject has no evidence of prior or active choroidal neovascularization (CNV). In some embodiments, the subject has evidence of prior or active choroidal neovascularization (CNV). In some embodiments, the subject has one or more disease features suggesting the subject is at risk for CNV such as sub- or intra-retinal fluid, sub-RPE fluid, non-exudative CNV, and a double-layer sign. In some embodiments, the subject has one or more well-demarcated GA lesions of a total area of about 2 to about 20 mm² in one or more eye, about 0.2 to 2.0 to about 20 mm² using en face imaging in one or more eye, and/or a diameter of below about 200 μm, between about 200 and about 250 μm, or greater than about 250 μm, optionally measured using cross-sectional OCT. In some embodiments, the subject has a best-corrected visual acuity score of greater than about 35 letters or a Snellen VA equivalent of about 20/400 or better. In some embodiments, the subject has a GA lesion of less than one disc area up to more than 10 disc areas. In some embodiments, the subject has a GA lesion of less than about 200 μm diameter up to more than about 500 μm.

In various aspects, the present disclosure relates to a method for treating or preventing an ocular disease or disorder, comprising administering to a subject in need thereof a pharmaceutical composition comprising an effective amount of one or more compounds of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, and a pharmaceutically acceptable carrier or excipient. In some embodiments, the ocular disease or disorder is a diabetic eye disease. In some embodiments, the ocular disease or disorder is a cataract or glaucoma. In some embodiments, the diabetic eye disease is diabetic retinopathy or diabetic macular edema (DME). In some embodiments, the subject has type 1 or type 2 diabetes. In some embodiments, the subject has a blood glucose level that exceeds normal levels. In some embodiments, the method further comprises reducing NFkb translocation to the nucleus in eye cells of the subject.

In various aspects, the present disclosure relates to a method for treating or preventing an ocular disease or disorder, comprising administering to a subject in need thereof a pharmaceutical composition comprising an effective amount of one or more compounds of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, and a pharmaceutically acceptable carrier or excipient. In some embodiments, the method further comprises reducing translocation of, or activity of, nuclear receptors, to or within the nucleus in the eye cells of the subject, wherein reducing translocation or activity results in the alteration of transcriptional activity. In some embodiments, the method further comprises normalizing cellular stress in cells under hypoxic, oxidative, superoxide, nitrosyl or glycemic stress. In some embodiments, the method further comprises administering one or more additional therapeutic agents. In some embodiments, the one or more additional therapeutic agents is selected from an anti-factor D antibody (e.g. lampalizumab (Genentech)), an anti-β-amyloid (anti-AB) antibody (e.g. GSK933776 (GSK)), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), doxycycline (ORACEA), emixustat hydrochloride, sirolimus (RAPAMUNE), and glatiramer acetate (COPAXONE). In some embodiments, the one or more additional therapeutic agents is selected from an anti-VEGF agent, an ACE inhibitor, a PPAR-gamma agonist or partial agonist, a renin inhibitor, a steroid, an agent that modulates nuclear receptor activity or gene transcription, a rapalogue, metformin or analogues thereof, an agent that modulates autophagy, a semapimod, a MIF inhibitor, a CCR2 inhibitor, CKR-2B, a 2-thioimidazole, CAS 445479-97-0, CCX140, clodronate, a 12indarit12e-liposome preparation and gadolinium chloride. In some embodiments, the additional therapeutic agent is one or more of an anti-vascular endothelial growth factor (VEGF) agent, a modulator of the complement cascade, an angiotensin-converting enzyme (ACE) inhibitor, a peroxisome proliferator-activated receptor (PPAR)-gamma agonist, a renin inhibitor, a corticosteroid, and an agent that modulates autophagy.

In embodiments, the one or more additional therapeutic agents is a nutraceutical agent, optionally selected from resveratrol, quercetin, taurine, carnosine, curcumin, glycyrrhizin, naringin, vitamin A, vitamin B, vitamin C, vitamin E, folate, and vitamin D.

In embodiments, the one or more additional therapeutic agents is a fatty acid agent, optionally selected from polyunsaturated fatty acid (PUFA), monounsaturated fatty acid (MUFA), or a saturated fatty acid (SFA).

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### BRIEF DESCRIPTION OF THE FIGURES

FIG. 1 is a graph of experimental data demonstrating MCP-1 (ccl2) mRNA expression in vitro following LPS stimulation of THP-1 macrophages treated with bindarit and bindarit ethyl ester. A=undifferentiated THP-1 monocytes. B=undifferentiated THP-1 monocytes exposed to LPS. C=THP-1 monocytes differentiated to macrophages (MO) with PMA. D=differentiated, PMA-exposed THP-1 MOs, exposed to LPS. E=differentiated, PMA-exposed THP-1 MOs, exposed to LPS and treated with bindarit. F=differentiated, PMA-exposed THP-1 MOs, exposed to LPS and treated with bindarit ethyl ester.

FIG. 2 is an image of a ¹H NMR spectrum of the bindarit-resveratrol conjugate (formula (V)).

FIG. 3 is an image of a mass spectroscopy spectrum of the bindarit-resveratrol conjugate (formula (V)).

FIG. 4 is an image of a ¹H NMR spectrum of bindarit-NHS ester in CDCl3.

FIG. 5 is an image of an overlay of the bindarit ¹H NMR spectrum and the bindarit-NHS ester 1H NMR spectrum. *=bindarit; +=bindarit-NHS ester

FIG. 6 is an image of a ¹H NMR spectrum of the bindarit-taurine conjugate (formula (VI) in D2O.

FIG. 7 is an image of a ¹H NMR spectrum of the bindarit-taurine conjugate (formula (VI)) in DMSO.

FIG. 8 is an image of a ¹H NMR spectrum of the bindarit-carnosine conjugate (formula (VIla) in D2O.

FIG. 9 is an image of a ¹H NMR spectrum of the bindarit-carnosine conjugate (formula (VIIa) in DMSO.

FIG. 10 is an image of an overlay of the bindarit-taurine conjugate ¹H NMR spectrum in D20 before and after acid hydrolysis (mol ratio-set sum of integrals to 1).

FIGS. 11A-11B are images of the bindarit-taurine conjugate ¹H NMR spectrum in D2O after enzymatic (esterase) hydrolysis showing 17% hydrolysis (mol %) (FIG. 11A), and showing broadening of bindarit-taurine conjugate peaks (FIG. 11B).

FIG. 12 is an image of an overlay of the bindarit-carnosine conjugate ¹H NMR spectrum in D2O before and after acid hydrolysis (mol ratio-set sum of integrals to 1).

### DETAILED DESCRIPTION

The present disclosure relates to, in part, the surprising finding that conjugates of bindarit and analogues thereof have immunomodulatory effects that makes them an effective agent for ocular diseases or disorders, such as dry AMD and RPD including, for example, protecting the retinal pigment epithelium (RPE).

In various aspects, the present disclosure provides for a method for treating or preventing any of the diseases or disorders described herein, including diabetic blinding eye diseases, dry AMD and/or RPD. In some embodiments, one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIIa), formula (VIIb), and formulas 1001 to 1005, or a pharmaceutically acceptable salt thereof, is also administered with an additional therapeutic agent. In various embodiments, the compounds effective for treating an ocular disease or disorder described herein is administered to a patient undergoing treatment with one or more additional therapeutic agents. Additional therapeutic agents, include, for example, one or more of an anti-VEGF agent, an ACE inhibitor, a PPAR-gamma agonist or partial agonist, a renin inhibitor, a steroid, and an agent that modulates autophagy, as well as a semapimod, a MIF inhibitor, a CCR2 inhibitor, CKR-2B, a 2-thioimidazole, CAS 445479-97-0, CCX140, clodronate, a clodonate-liposome preparation or gadolinium chloride.

### Compounds of the Disclosure

In one aspect, the present disclosure is related to a compound of formula (I), or a pharmaceutically acceptable salt thereof:

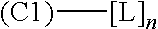

wherein in formula (I):
C1 is bindarit or an analogue thereof;
L is independently at each occurrence a linker covalently linked to C1, wherein each L is optionally further covalently linked to an active agent C2, and each occurrence of C2 may be the same or different; and
n is an integer selected from 1 to 4.

Bindarit is an inhibitor of MCP-1 production in vitro and in vivo and, without wishing to be bound by theory, its beneficial effects in animal models of inflammation may be related to this anti-MCP-1 activity (see Mirolo, et al. Eur Cytokine Netw 2008; 19:119-122). Bindarit has also been shown to selectively inhibit the production of MCP-2 and MCP-3 (see Mirolo, et al. Eur Cytokine Netw 2008; 19:119-122).

In some embodiments, C1 is a radical formed from bindarit or the analogue thereof, and each L is covalently linked to C1.

In some embodiments, C1 comprises a group selected from:

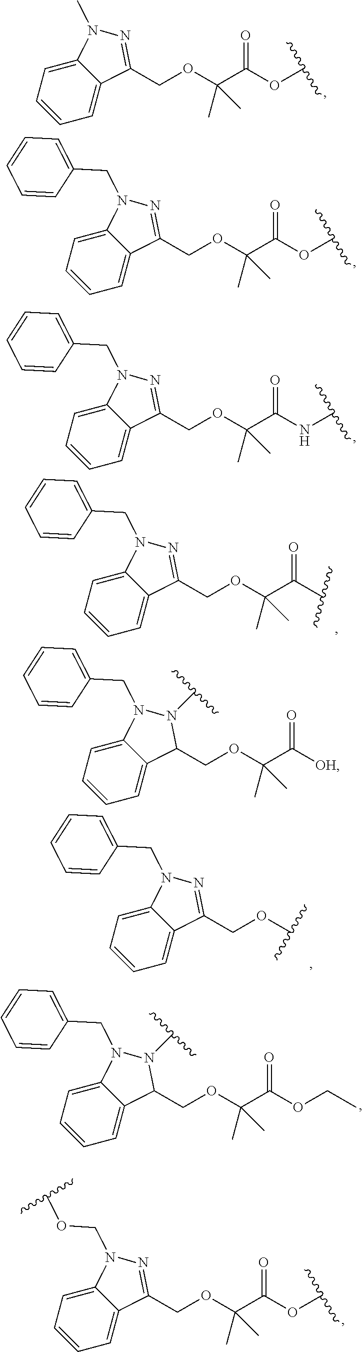

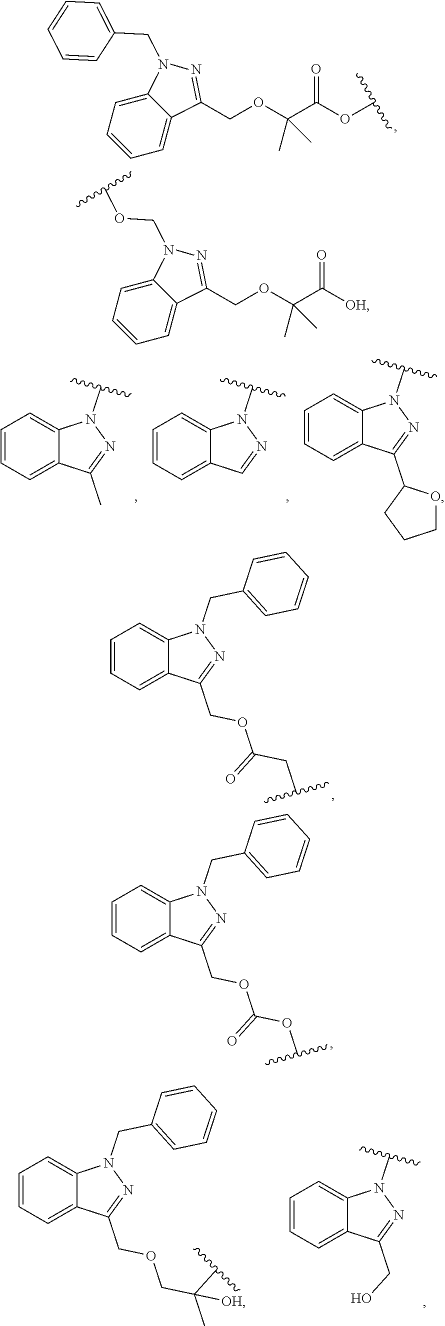

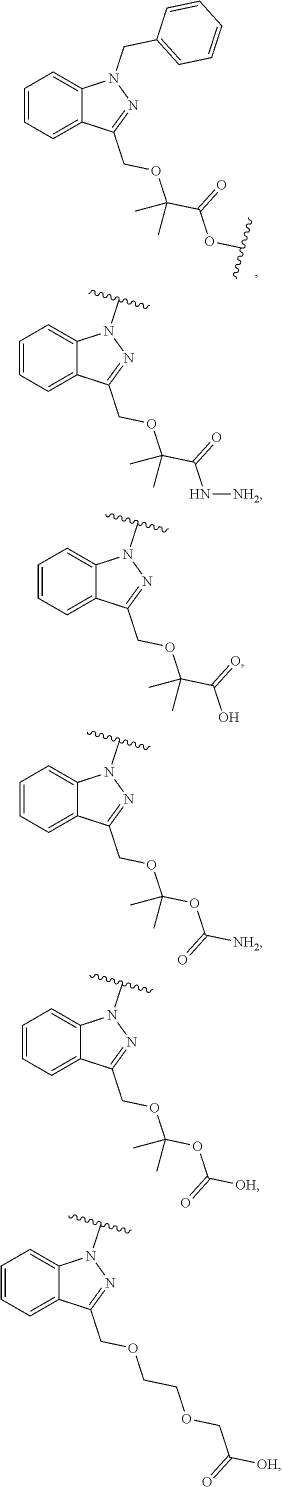

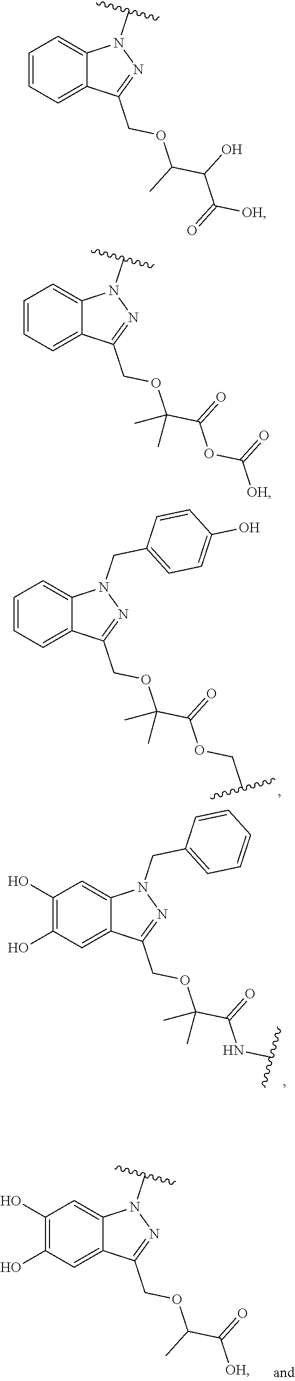

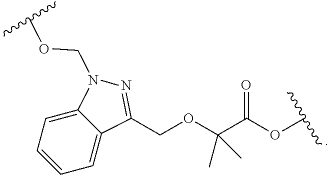 [SMILES: *OCn1nc(COC(C)(C)C(=O)O*)c2ccccc21]

In some embodiments, the compound of formula (I) is a compound of formula (Iia):

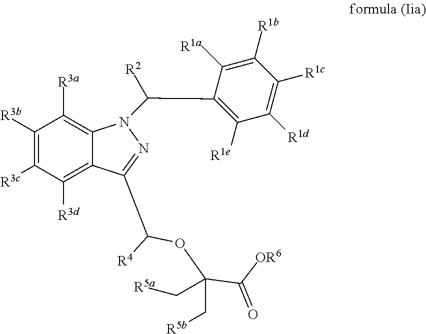

wherein in formula (Iia):
one to four of Ria, R^{1b}, R^{1c}, R^{1d}, R^{1e}, R², R^{3a}, R³⁶, R^{3c}, R^{3d}, R⁴, R^{5a}, R^{5b}, and R⁶ each independently represents -L or -L-C2 when L is further covalently linked to C2, and the remaining R^{1a}, R^{1b}, R^{1c}, R^{1d}, R^{1e}, R², R^{3a}, R^{3b}, R^{3c}, R^{3d}, R⁴, R^{5a}, R^{5b}, and R⁶ are independently selected at each occurrence from hydrogen, deuterium, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O), R^{a} (where t is 1 or 2), —S(O), R^{a} (where t is 1 or 2), —S(O)OR^{a} (where t is 1 or 2), —S(O)ₜN(R^{a})₂ (where t is 1 or 2), and PO₃(R^{a})₂;
L Is independently at each occurrence a linker, wherein each L is optionally further covalently linked to an active agent C2, and each occurrence of C2 may be the same or different; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

In some embodiments, the compound of formula (I) is a compound of formula (Iic):

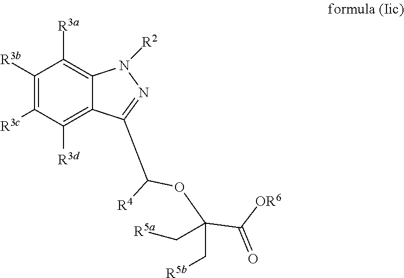

wherein in formula (Iic):
one to four of R², R^{3a}, R^{3b}, R^{3c}, R^{3d}, R⁴, R^{5a}, R^{5b}, and R⁶ each independently represents -L or -L-C2 when L is further covalently linked to C2, and the remaining R², R^{3a}, R^{3b}, R^{3c}, R^{3d}, R⁴, R^{5a}, R^{5b}, and R⁶ are independently selected at each occurrence from hydrogen, deuterium, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), and PO₃(R^{a}) 2;
L Is independently at each occurrence a linker, wherein each L is optionally further covalently linked to an active agent C2, and each occurrence of C2 may be the same or different; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

In some embodiments, C1 is bindarit ethyl ester (a.k.a. ethyl 2-((1-benzl-1H-indazol-3-yl)methoxy)-2-methylpropanoate) or an analogue thereof. In some embodiments, C1 is a radical formed from bindarit ethyl ester or an analogue thereof, and each L is covalently linked to C1. In a non-limiting embodiment, bindari ethyl ester is a useful intermediate for preparing C1 and/or compounds of formula (I). Non-limiting examples of methods for preparing bindarit ethyl ester can be found in WO2020261158, which is incorporated by reference herein in its entirety. The structure of bindarit ethyl ester (a.k.a. ethyl 2-((1-benzl-1H-indazol-3-yl)methoxy)-2-methylpropanoate) is shown below:

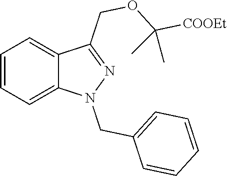 [SMILES: CCOC(=O)C(C)(C)OCc1nn(Cc2ccccc2)c2ccccc12]

In some embodiments, the compound of formula (I) is a compound of formula (iIb):

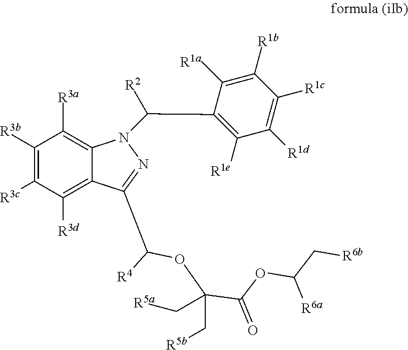

wherein in formula (iIb):
one to four of R^{1a}, R^{1b}, R^{1c}, R^{1d}, R^{1e}, R², R^{3a}, R^{3b}, R^{3c}, R^{3d}, R⁴, R^{5a}, R^{5b}, R^{6a}, and R^{6b} each independently represents -L or -L-C2 when L is further covalently linked to C2, and the remaining R^{1a}, R^{1b}, R^{1c}, R^{1d}, R^{1e}, R², R^{3a}, R^{3b}, R^{3c}, R^{3d}, R⁴, R^{5a}, R^{5b}, R^{6a}, and R^{6b} are independently selected at each occurrence from hydrogen, deuterium, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O), R^{a} (where t is 1 or 2), —S(O), R^{a} (where t is 1 or 2), —S(O)OR^{a} (where t is 1 or 2), —S(O)ₜN(R^{a}) 2 (where t is 1 or 2), and PO₃(R^{a}) 2;
L is independently at each occurrence a linker, wherein each L is optionally further covalently linked to an active agent C2, and each occurrence of C2 may be the same or different; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

In some embodiments, the compound of formula (I) is a compound of formula (i2a):

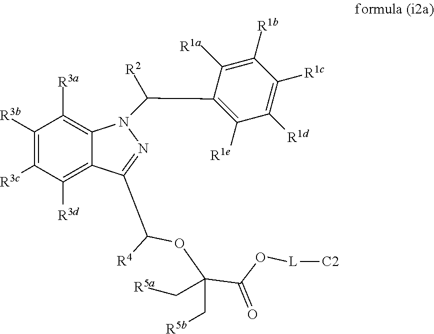

wherein in formula (i2a):
R^{1a}, R^{1b}, R^{1c}, R^{1d}, R^{1e}, R², R^{3a}, R^{3b}, R^{3c}, R^{3d}, R⁴, R^{5a}, and R^{5b} are independently selected at each occurrence from hydrogen, deuterium, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), PO₃(R^{a})₂, -L, and -L-C2 when L is further covalently linked to C2;
L is independently at each occurrence a linker, wherein each Lis optionally further covalently linked to an active agent C2;
C2 is an active agent, wherein each occurrence of C2 may be the same or different; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

In some embodiments, the compound of formula (I) is a compound of formula (i2b):

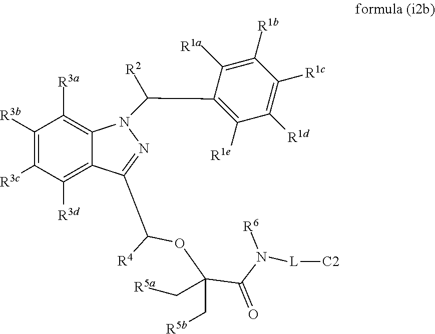

wherein in formula (i2a):
R^{1a}, R^{1b}, R^{1c}, R^{1d}, R^{1e}, R², R^{3a}, R^{3b}, R^{3c}, R^{3d}, R⁴, R^{5a}, R^{5b} and R⁶ are independently selected at each occurrence from hydrogen, deuterium, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O) &R^{a} (where is 1 or 2), —S(O), R^{a} (where t is 1 or 2), —S(O)OR^{a} (where t is 1 or 2), —S(O) \N(R^{a}) 2 (where t is 1 or 2), PO₃(R^{a})₂, -L, and -L-C2 when L is further covalently linked to C2;
L is independently at each occurrence a linker, wherein each L is optionally further covalently linked to an active agent C2;
C2 is an active agent, wherein each occurrence of C2 may be the same or different; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

In some embodiments, in formula (Iia), R^{1a}, R^{1b}, R^{1c}, R^{1d}, and Rie are each hydrogen. In some embodiments, in formula (Iia), R² is hydrogen. In some embodiments, in formula (Iia), R^{3a}, R^{3b}, R^{3c}, and R^{3d} are each hydrogen. In some embodiments, in formula (Iia), R⁴ is hydrogen. In some embodiments, in formula (Iia), R^{5a} and R^{5b} are each hydrogen. In some embodiments, in formula (Iia), R⁶ is -L. In some embodiments, in formula (Iia), R⁶ is -L-C2. In some embodiments, in formula (Iia), C2 is selected from resveratrol, carnosine, and taurine. In some embodiments, in formula (Iia), L is a single bond and C2 is selected from resveratrol, carnosine, and taurine. In some embodiments, in formula (Iia), C2 is selected from carnosine and taurine. In some embodiments, in formula (lia), L is a single bond and C2 is selected from carnosine and taurine. In some embodiments, in formula (Iia), C2 comprises carnosine. In some embodiments, in formula (Iia), L is a single bond and C2 comprises carnosine. In some embodiments, in formula (Iia), C2 comprises taurine. In some embodiments, in formula (Iia), L is a single bond and C2 comprises taurine. In some embodiments, in formula (Iia), L is a single bond and C2 comprises resveratrol. In some embodiments, in formula (Iia), L is a single bond and C2 comprises resveratrol. In some embodiments, in formula (Iia), C2 is a radical of resveratrol, taurine, or carnosine. In some embodiments, in formula (lia), C2 is a radical of resveratrol. In some embodiments, in formula (Iia), C2 is a radical of taurine. In some embodiments, in formula (Iia), C2 is a radical of carnosine. In some embodiments, in formula (Iia), L is a single bond and C2 is a radical of resveratrol, taurine, or carnosine. In some embodiments, in formula (Iia), L is a single bond and C2 is a radical of resveratrol. In some embodiments, in formula (Iia), L is a single bond and C2 is a radical of taurine. In some embodiments, in formula (Iia), L is a single bond and C2 is a radical of carnosine.

In some embodiments, in formula (iIb), R^{1a}, R^{1b}, R^{1c}, R^{1d}, and Rie are each hydrogen. In some embodiments, in formula (iIb), R² is hydrogen. In some embodiments, in formula (iIb), R^{3a}, R^{3b}, R^{3c}, and R^{3d} are each hydrogen. In some embodiments, in formula (iIb), R⁴ is hydrogen. In some embodiments, in formula (iIb), R^{5a} and R^{5b} are each hydrogen. In some embodiments, in formula (iIb), R^{6a} and R^{6b} are each hydrogen. In some embodiments, in formula (iIb), R^{6a} is -L. In some embodiments, in formula (iIb), R^{6a} is -L-C2. In some embodiments, in formula (iIb), R^{6b} is -L. In some embodiments, in formula (iIb), R^{6b} is -L-C2. In some embodiments, in formula (iIb), R^{6a} is -L and R^{6b} is hydrogen. In some embodiments, in formula (iIb), R^{6a} is -L-C2 and R^{6b} is hydrogen. In some embodiments, in formula (iIb), R^{6a} is hydrogen and R^{6b} is -L. In some embodiments, in formula (iIb), R^{6a} is hydrogen and R^{6b} is -L-C2. In some embodiments, in formula (Iib), C2 is selected from resveratrol, carnosine, and taurine. In some embodiments, in formula (Iib), L is a single bond and C2 is selected from resveratrol, carnosine, and taurine. In some embodiments, in formula (Iib), C2 is selected from carnosine and taurine. In some embodiments, in formula (Iib), L is a single bond and C2 is selected from carnosine and taurine. In some embodiments, in formula (Iib), C2 comprises carnosine. In some embodiments, in formula (Iib), L is a single bond and C2 comprises carnosine. In some embodiments, in formula (Iib), C2 comprises taurine. In some embodiments, in formula (Iib), L is a single bond and C2 comprises taurine. In some embodiments, in formula (Iib), L is a single bond and C2 comprises resveratrol. In some embodiments, in formula (Iib), L is a single bond and C2 comprises resveratrol. In some embodiments, in formula (Iib), C2 is a radical of resveratrol, taurine, or carnosine. In some embodiments, in formula (Iib), C2 is a radical of resveratrol. In some embodiments, in formula (Iib), C2 is a radical of taurine. In some embodiments, in formula (Iib), C2 is a radical of carnosine. In some embodiments, in formula (lib), L is a single bond and C2 is a radical of resveratrol, taurine, or carnosine. In some embodiments, in formula (lib), L is a single bond and C2 is a radical of resveratrol. In some embodiments, in formula (Iib), L is a single bond and C2 is a radical of taurine. In some embodiments, in formula (Iib), L is a single bond and C2 is a radical of carnosine.

In some embodiments, in formula (Iic), R² is hydrogen. In some embodiments, in formula (Iic), R^{3a}, R^{3b}, R^{3c}, and R^{3d} are each hydrogen. In some embodiments, in formula (Iic), R⁴ is hydrogen. In some embodiments, in formula (Iic), R^{5a} and R^{5b} are each hydrogen. In some embodiments, in formula (Iic), R⁶ is -L. In some embodiments, in formula (Iic), R⁶ is -L-C2. In some embodiments, in formula (Iic), C2 is selected from resveratrol, carnosine, and taurine. In some embodiments, in formula (Iic), L is a single bond and C2 is selected from resveratrol, carnosine, and taurine. In some embodiments, in formula (Iic), C2 is selected from carnosine and taurine. In some embodiments, in formula (Iic), L is a single bond and C2 is selected from carnosine and taurine. In some embodiments, in formula (Iic), C2 comprises carnosine. In some embodiments, in formula (Iic), L is a single bond and C2 comprises carnosine. In some embodiments, in formula (Iic), C2 comprises taurine. In some embodiments, in formula (Iic), L is a single bond and C2 comprises taurine. In some embodiments, in formula (Iic), L is a single bond and C2 comprises resveratrol. In some embodiments, in formula (Iic), L is a single bond and C2 comprises resveratrol. In some embodiments, in formula (Iic), C2 is a radical of resveratrol, taurine, or carnosine. In some embodiments, in formula (Iic), C2 is a radical of resveratrol. In some embodiments, in formula (Iic), C2 is a radical of taurine. In some embodiments, in formula (Iic), C2 is a radical of carnosine. In some embodiments, in formula (lic), L is a single bond and C2 is a radical of resveratrol, taurine, or carnosine. In some embodiments, in formula (lic), L is a single bond and C2 is a radical of resveratrol. In some embodiments, in formula (Iic), L is a single bond and C2 is a radical of taurine. In some embodiments, in formula (Iic), L is a single bond and C2 is a radical of carnosine.

In some embodiments, in formula (i2a), R^{1a}, R^{1b}, R^{1c}, R^{1d}, and Rie are each hydrogen. In some embodiments, in formula (i2a), R² is hydrogen. In some embodiments, in formula (i2a), R^{3a}, R^{3b}, R^{3c}, and R^{3d} are each hydrogen. In some embodiments, in formula (12a), R⁴ is hydrogen. In some embodiments, in formula (i2a), R^{5a} and R^{5b} are each hydrogen. In some embodiments, in formula (i2a), L is a single bond. In some embodiments, in formula (i2a), C2 is selected from resveratrol, carnosine, and taurine. In some embodiments, in formula (i2a), L is a single bond and C2 is selected from resveratrol, carnosine, and taurine. In some embodiments, in formula (i2a), C2 comprises carnosine. In some embodiments, in formula (i2a), L is a single bond and C2 comprises carnosine. In some embodiments, in formula (i2a), C2 comprises taurine. In some embodiments, in formula (i2a), L is a single bond and C2 comprises taurine. In some embodiments, in formula (i2a), L is a single bond and C2 comprises resveratrol. In some embodiments, in formula (12a), L is a single bond and C2 comprises resveratrol. In some embodiments, in formula (i2a), C2 is a radical of resveratrol, taurine, or carnosine. In some embodiments, in formula (i2a), C2 is a radical of resveratrol. In some embodiments, in formula (i2a), C2 is a radical of taurine. In some embodiments, in formula (i2a), C2 is a radical of carnosine. In some embodiments, in formula (i2a), L is a single bond and C2 is a radical of resveratrol, taurine, or carnosine. In some embodiments, in formula (i2a), L is a single bond and C2 is a radical of resveratrol. In some embodiments, in formula (12a), L is a single bond and C2 is a radical of taurine. In some embodiments, in formula (i2a), L is a single bond and C2 is a radical of carnosine.

In some embodiments, in formula (i2b), R^{1a}, R^{1b}, R^{1c}, R^{1d}, and Rie are each hydrogen. In some embodiments, in formula (i2b), R² is hydrogen. In some embodiments, in formula (i2a), R^{3a}, R^{3b}, R^{3c}, and R^{3d} are each hydrogen. In some embodiments, in formula (i2b), R⁴ is hydrogen. In some embodiments, in formula (i2a), R^{5a} and R^{5b} are each hydrogen. In some embodiments, in formula (i2b), R⁶ is hydrogen or optionally substituted alkyl. In some embodiments, in formula (i2b), R⁶ is hydrogen. In some embodiments, in formula (i2a), L is a single bond. In some embodiments, in formula (i2b), C2 is selected from resveratrol, carnosine, and taurine. In some embodiments, in formula (i2b), L is a single bond and C2 is selected from resveratrol, carnosine, and taurine. In some embodiments, in formula (i2b), C2 is selected from carnosine and taurine. In some embodiments, in formula (i2b), L is a single bond and C2 is selected from carnosine and taurine. In some embodiments, in formula (i2b), C2 comprises carnosine. In some embodiments, in formula (i2b), L is a single bond and C2 comprises carnosine. In some embodiments, in formula (i2b), C2 comprises taurine. In some embodiments, in formula (i2b), L is a single bond and C2 comprises taurine. In some embodiments, in formula (i2b), L is a single bond and C2 comprises resveratrol. In some embodiments, in formula (i2b), L is a single bond and C2 comprises resveratrol. In some embodiments, in formula (i2b), C2 is a radical of resveratrol, taurine, or carnosine. In some embodiments, in formula (i2b), C2 is a radical of resveratrol. In some embodiments, in formula (i2b), C2 is a radical of taurine. In some embodiments, in formula (i2b), C2 is a radical of carnosine. In some embodiments, in formula (i2b), L is a single bond and C2 is a radical of resveratrol, taurine, or carnosine. In some embodiments, in formula (i2b), L is a single bond and C2 is a radical of resveratrol. In some embodiments, in formula (i2b), L is a single bond and C2 is a radical of taurine. In some embodiments, in formula (i2b), L is a single bond and C2 is a radical of carnosine.

In some embodiments, the compound of formula (I) is a compound of formula (Ia), formula (Ib), formula (Ic), formula (Id), formula (Ie), formula (Ia1), formula (Ia2), or formula (Ia3), or a pharmaceutically acceptable salt thereof:

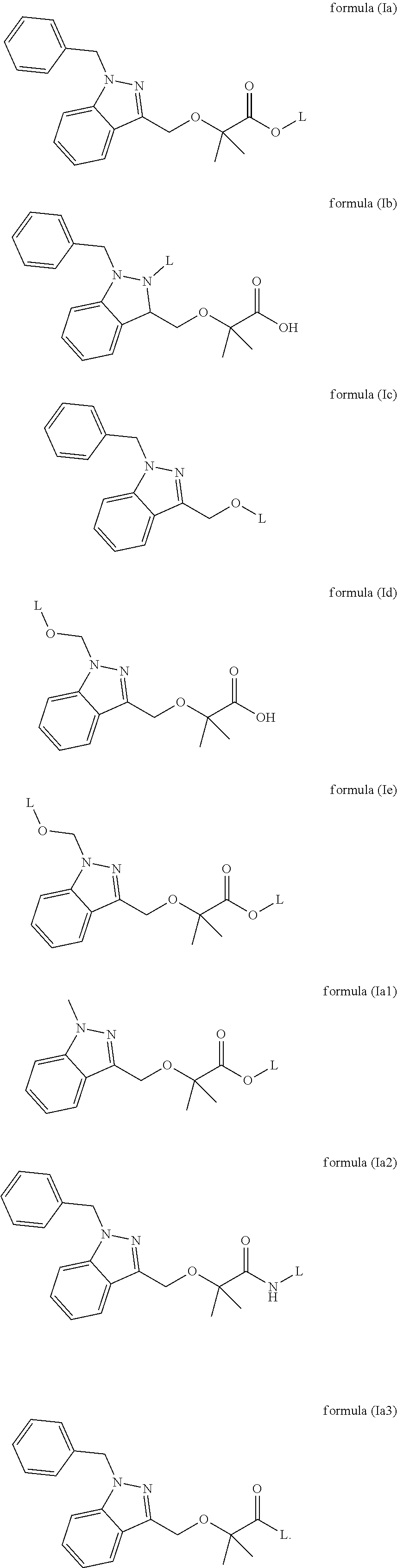

In one aspect, each occurrence of linker L may be the same or different. In some embodiments, the linker is a cleavable linker. In some embodiments, the cleavable linker is a chemically labile linker. Non-limiting examples of chemically labile linkers include acid cleavable (pH sensitive) linkers (e.g. hydrazone) and enzyme cleavable linkers (glutathione-sensitive) (e.g. disulfide linkers). In some embodiments, the cleavable linker is an enzyme cleavable linker. Non-limiting examples of enzyme cleavable linkers include peptide-based linkers (protease-sensitive) and L-glucuronide linkers (glucuronidase-sensitive). In some embodiments, the linker is a non-cleavable linker. In some embodiments, the linker is difunctional. In some embodiments, the linker is trifunctional. In some embodiments, the linker is a crosslinker. In some embodiments, the linker is hydrophilic. In some embodiments, the linker is hydrophobic. In some embodiments, the linker is homofunctional. In some embodiments, the linker is heterofunctional (contains diverse functional groups). In some embodiments the linker is or comprises an ester, or ester chain.

In some embodiments, the linker comprises one or more polyesters. Non-limiting examples of polyesters include polylactic acids, poly-L-lactic acid (PLLA), and a random copolymer of L-lactic acid and D-lactic acid, and derivatives thereof, polycaprolactone, polyhydroxybutyric acid, polyhydroxyvaleric acid, polyethylene succinate, polybutylene succinate, polybutylene adipate, polymalic acid, polyglycolic acid, polysuccinate, polyoxalate, polybutylene diglycolate, and polydioxanone. In some embodiments, the linker comprises polypropylene.

In some embodiments, the linker comprises one or more polyolefins. Non-limiting examples of polyolefins include polypropylenes, ethylene vinyl acetate, ethylene methyl acrylate, polyethylene, poly(I-butene), polystyrene, poly(2-butene), poly(l-pentene), poly(2-pentene), poly(3-methyl-1-pentene), poly(4-methyl-1-pentene), 1,2-poly-1,3-butadiene, 1,4-poly-1,3-butadiene, polyisoprene, polychloroprene, poly(vinyl acetate), poly(vinylidene chloride), poly(tetrafluoroethylene) (PTFE), poly(vinylidene fluoride) (PVDF), acrylonitrile-butadiene-styrene (ABS), or mixtures thereof.

In some embodiments, the linker comprises one or more of a primary amine, a sulfhydryl(thiol), an acid, an alcohol, or a bromide. In some embodiments, the linker is in part a linear linker. In some embodiments, the linker a linear linker. Linear linkers comprise groups including, but not limited to, methylenic or polymeric chain groups. In some embodiments, the linker is in part a non-linear linker. In some embodiments, the linker is a non-linear linker. Non-linear linkers comprise groups including, but not limited to, aromatic, heteroaromatic or non-aromatic cycle groups. In some embodiments, the linker comprises at least one protecting group. Non-limiting examples of protecting groups include a Boc group, a Fmoc group, and a Cbz group.

In some embodiments, the linker comprises one or more amino acids. In some embodiments, the linker comprises about 1, about 2, about 3, about 4, or about 5 or more amino acids. In some embodiments, the linker comprises one or more tripeptides. In a non-limiting embodiment, a tripeptide comprises three amino acids, which may each be the same or different. Non-limiting examples of tripeptides include tripeptides comprising and/or consisting of proline, serine, and alanine (PSA), and tripeptides comprising and/or consisting of arginine, glycine, and aspartic acid (RGD).

In some embodiments, each L independently comprises one or more of a bond, —(CH₂CH₂O)—, —C(O), —(CH₂)—, —O—, —C(O)O—, —OC(O)—, —C(O)NR^{a}—, —NR^{a}C, (O)—, —S—, —S—S—, —SCH₂CH₂S—, —NR^{a}—, —S(O)—, —S(O)₂—, —CR^{a}₂—, —C(O)S—, —SC(O)—, —C(O)NR^{a}SO₂—, —SO₂NR^{a}C, (O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

In some embodiments, L is covalently linked to C1 and/or to C2 via one or more ester, carbonate, carbonate ester, or anhydride linkages.

In other embodiments, L is covalently linked to C1 and/or to C2 via one or more carbonate linkages. In other embodiments, L is covalently linked to C1 and/or to C2 via one or more ester linkages.

In some embodiments, L comprises a group selected from —C(O)O—(R)—OC(O)—, —C(O)—(R^{x})—C(O)—, or —O—(R^{x})—O—; wherein R^{x} is a radical of a polyol and includes at least one free hydroxyl group or R^{x} is C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, —(CH₂CH₂O), CH₂CH₂—, —(CH₂CH₂CH₂CH₂O)ₜCH₂CH₂CH₂CH₂—, or —(CH₂CH(CH₃)O)₂CH₂CH(CH₃)—; and q, r, and s are each independently integers from 1 to 10 (e.g., 1 to 10, 1 to 5, or 5 to 10).

In some embodiments, each L independently comprises one or more of a PEG-based linker, a polyester, polypropylene, polyolefin, polyurethane, alhydrogel, vitamin K, caffeine, menadione, benzoic acid, cholesterol, PVP, β-cyclodextrin,

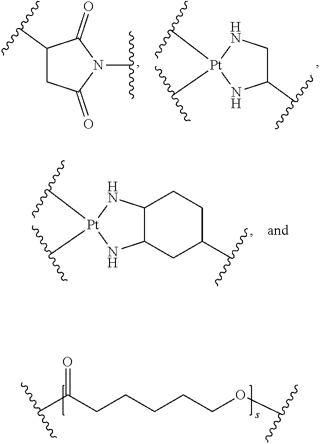

wherein s is 1 to 5.

In some embodiments, the linker comprises one or more polyethers. Non-limiting examples of polyethers include poly(propylene glycol) (PEG), polytetrahydrofuran, and copolymers of poly(ethylene glycol) and poly(propylene glycol).

In some embodiments, L is a PEG-based linker comprising one or more PEG molecules. In some embodiments, PEG has an average molecular weight of from about 10 kDa to about 400 kDa. In some embodiments, the polyethylene glycols, which are suitable for use in the present disclosure are those having an average molecular weight of at least 10,000 daltons to 50,000 daltons. In some embodiments, the PEG molecules have an average molecular weight of about 30,000 daltons to about 50,000 daltons, for example, about 32,000 daltons to about 48,000 daltons, about 34,000 daltons to about 46,000 daltons, about 36,000 daltons to about 44,000 daltons, about 43,000 daltons to about 45,000 daltons, or about 38,000 daltons to about 42,000 daltons. In some embodiments, the PEGs have an average molecular weight of 20,000 daltons, such as an average molecular weight of in the range of 20,000 to 700,000 daltons, for example in the range of 20,000 to 600,000 daltons, such as in the range of 35,000 to 500,000 daltons, for example in the range of 35,000 to 400,000 daltons, such as in the range of 35,000 to 350,000 daltons, for example in the range of 50,000 to 350,000 daltons, such as in the range of 100,000 to 300,000 daltons, for example in the range of 150,000 to 350,000 daltons, such as in the range of 200,000 to 300,000 daltons. In certain embodiments, polyethylene glycols suitable for use in the chimeric proteins described herein are those having an average molecular weight selected from approximately 10,000 daltons, approximately 15,000 daltons, approximately 20,000 daltons, approximately 25,000 daltons, approximately 30,000 daltons, approximately 35,000 daltons, approximately 40,000 daltons, approximately 41,000 daltons, approximately 42,000 daltons, approximately 43,000 daltons, approximately 44,000 daltons, approximately 45,000 daltons, approximately 50,000 daltons, approximately 75,000 daltons, approximately 100,000 daltons, approximately 150,000 daltons, approximately 200,000 daltons, approximately 250,000 daltons, approximately 300,000 daltons, approximately 400,000 daltons, 150,000 daltons, 200,000 daltons, 250,000 daltons, 300,000 daltons, 400,000 daltons. In the present context, referring to the average molecular weight of polyethylene glycols, “approximately” means+/−30%. In some embodiments, PEG, that is attached covalently to a compound of one or more of formula (I), formula (Ia), formula (Ib), formula (Ic), formula (Id), formula (Ie), formula (Ia1), formula (If), formula (Ig), formula (Ih), formula (Ii), formula (Ij), formula (Ik), formula (Iia), formula (iIb), formula (Iic), formula (IIIa), and formula (IIIb) described herein or PEG that is incorporated into a linker L has an average molecular weight of 10 kDa, 20 kDa, or 40 kDa. In some embodiments, the PEG that is attached covalently to the compounds described herein or PEG that is incorporated into a linker L, is a branched PEG, a star PEG, or a comb PEG.

In some embodiments, attachment of the PEG moiety increases the half-life and/or reduces the immunogenicity of the compound of formula (I), formula (Ia), formula (Ib), formula (Ic), formula (Id), formula (Ie), formula (Ia1), formula (If), formula (Ig), formula (Ih), formula (Ii), formula (Ij), formula (Ik), formula (Iia), formula (iIb), formula (Iic), formula (IIIa), or formula (IIIb). Generally, any suitable form of pegylation can be used, such as the pegylation used in the art for antibodies and antibody fragments (including but not limited to single domain antibodies such as VHHs); see, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO04060965, the entire contents of which are hereby incorporated by reference. Various reagents for pegylation of proteins are also commercially available, for example, from Nektar Therapeutics, USA. In some embodiments, site-directed pegylation is used, in particular via a cysteine-residue (see, for example, Yang et al., Protein Engineering, 16, 10, 761-770 (2003), the entire contents of which is hereby incorporated by reference). In some embodiments, the compound of formula (I), formula (Ia), formula (Ib), formula (Ic), formula (Id), formula (Ie), formula (la1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 is modified so as to suitably introduce one or more cysteine residues for attachment of PEG, or an amino acid sequence comprising one or more cysteine residues for attachment of PEG may be fused to an amino-and/or carboxy-terminus present on a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, using techniques known in the art.

In some embodiments, wherein the PEG-based linker comprises one or more of poly(ethylene glycol) (PEG), an amine reactive PEG-based linker (e.g. Bis-PEG-acid, Bis-PEG-NHS, Boc-PEG, Fmoc-PEG, PEG Acid, PEG Aldehyde, PEG NHS ester, PEG Phosphonate, PEG PFP ester, PEG Tosylate), a biotinylated PEG-based linker (e.g. PEG-biotin), a branched PEG-based linker; a reactive carbonyl PEG-based linker (e.g. aminooxy-PEG), a carboxyl and/or active ester reactive PEG-based linker (e.g. amine PEG), a click-reagent PEG-based linker (e.g. alkyne PEG, azide-PEG); a copper-free click chemistry PEG-based linker (e.g. DBCO-PEG, BCN-PEG); a Cu-catalyzed click chemistry PEG-based linker (e.g. propargyl-PEG); a fluorescent PEG labeling and homobifunctional PEG-based linker (e.g. bis-PEG-acid, bis-PEG-NHS, bis-PEG-PFP, bis-propargyl-PEG, amine-PEG-amine, azido-PEG-azide, bromo-PEG-bromide), a lipid PEG, a metal surface binding and thiol reactive PEG-based linker (e.g. Thiol PEG, Bromo-PEG, Mal PEG), PEG-pAsp, PEG-pGlu, PEG-b-PMPMC, and PEG-PGLA.

In some embodiments, L comprises one or more PEG-based linkers selected from triethylene glycol, tetraethylene glycol, pentaethylene glycol, hepatheylyene glycol, nonaethylene glycol, hexane, polyethylene glycol (MW=200), and polyethylene glycol (MW=300). In some embodiments, the PEG-based linker is selected from poly(ethylene glycol) methyl ether (MW=475), poly(ethylene glycol) diacrylate (PEGDA) (MW=258), poly(ethylene glycol) diacrylate (MW=575), (poly(ethylene glycol) diacrylate (MW=700), poly(ethylene glycol) dimethacrylate (PEGDMA) (MW=550), and poly(ethylene glycol) dimethacrylate (MW=750).

In some embodiments, L comprises a dithiane. Non-limiting examples of dithianes include D,L-dithiothreitol (DTT), 1,2-ethanedithiol (DET), 1,3-propanedithiol (DPT), and 1,4-butanedithiol (DBT).

In some embodiments, L comprises one or more groups selected from D,L-dithiothreitol (DTT), 1,2-ethanedithiol (DET), 1,3-propanedithiol (DPT), 1,4-butanedithiol (DBT), methyl acrylate (MA), methyl methacrylate (MMA), poly(ethylene glycol) methyl ether (MW=475), poly(ethylene glycol) diacrylate (PEGDA) (MW=258), poly(ethylene glycol) diacrylate (MW=575), (poly(ethylene glycol) diacrylate (MW=700), poly(ethylene glycol) dimethacrylate (PEGDMA) (MW=550), and poly(ethylene glycol) dimethacrylate (MW=750).

In some embodiments, L comprises a polymer. In some embodiments, the polymer is non-toxic. In some embodiments, the polymer is biodegradable. Non-limiting examples of polymers include polyethylene glycol (PEG), vinyl polymers (HPMA), polyurethane, and polyaminoacids.

In some embodiments, the polymer comprises a zwitterionic monomer. Non-limiting examples of zwitterionic monomer include HEMA-phosphorylcholine, PEG, biocompatible fatty acids and derivatives thereof, Hydroxy Alkyl Starch (HAS), Hydroxy Ethyl Starch (HES), Poly Ethylene Glycol (PEG), Poly(Glyx-Sery) (HAP), Hyaluronic acid (HA), Heparosan polymers (HEP), Fleximers, Dextran, Poly-sialic acids (PSA), Fc domains, Transferrin, 25 Albumin, Elastin like (ELP) peptides, XTEN polymers, PAS polymers, PA polymers, Albumin binding peptides, CTP peptides, and FcRn binding peptides.

In some embodiments, the polymer is a phosphorylcholme-based polymer. Non-limiting examples of phosphorylcholme-based polymer include poly(acryloyloxyethyl phosphorylcholine) and poly(methacryloyloxyethyl phosphorylcholine).

In some embodiments, L comprises one or more of a group selected from:

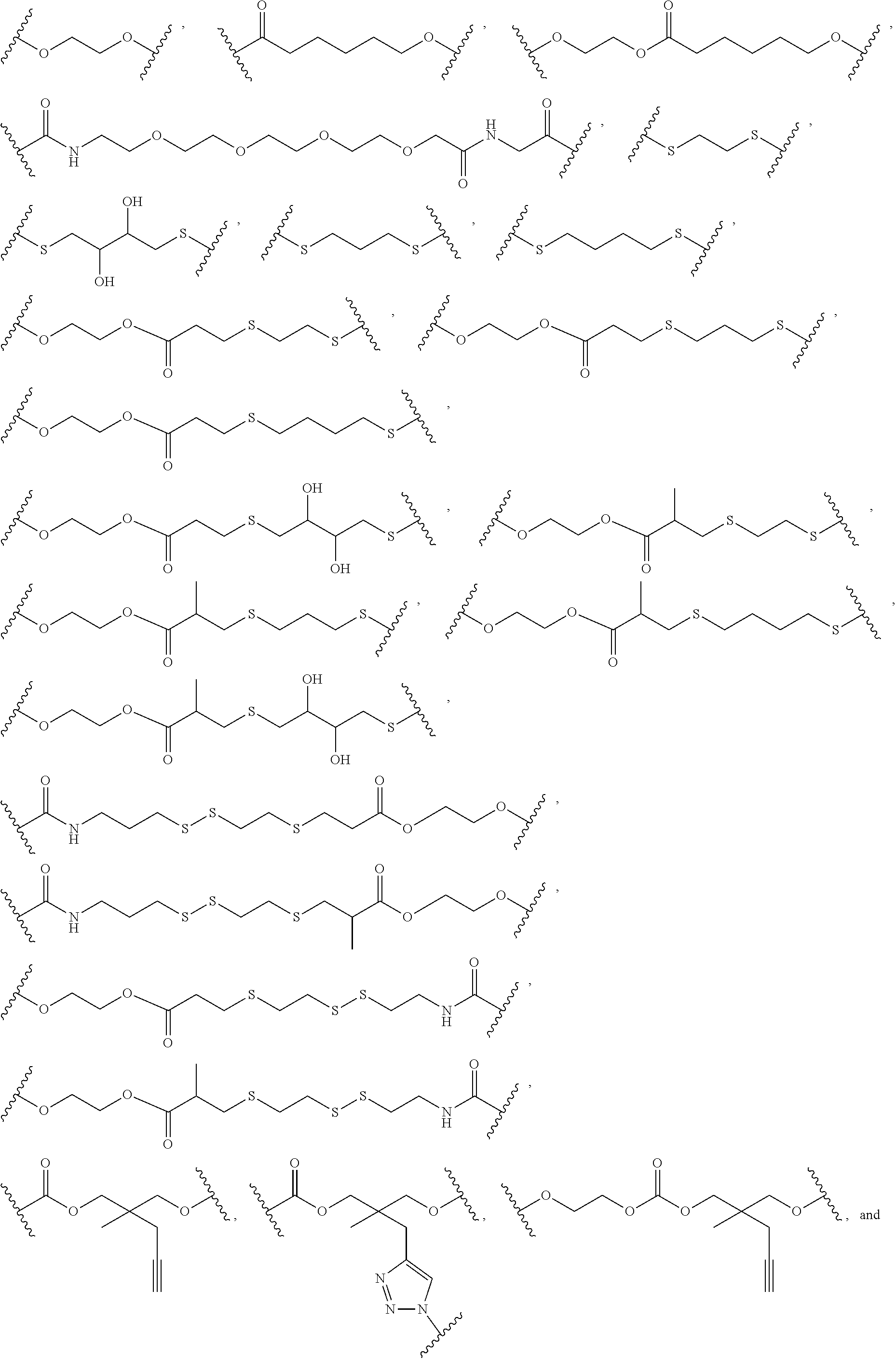

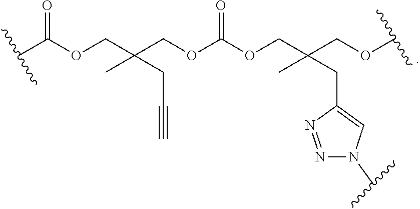 [SMILES: *OCC(C)(COC(=O)OCC(C)(CC#C)COC(*)=O)Cc1cn(*)nn1]

In some embodiments, the PEG-based linker is selected from:

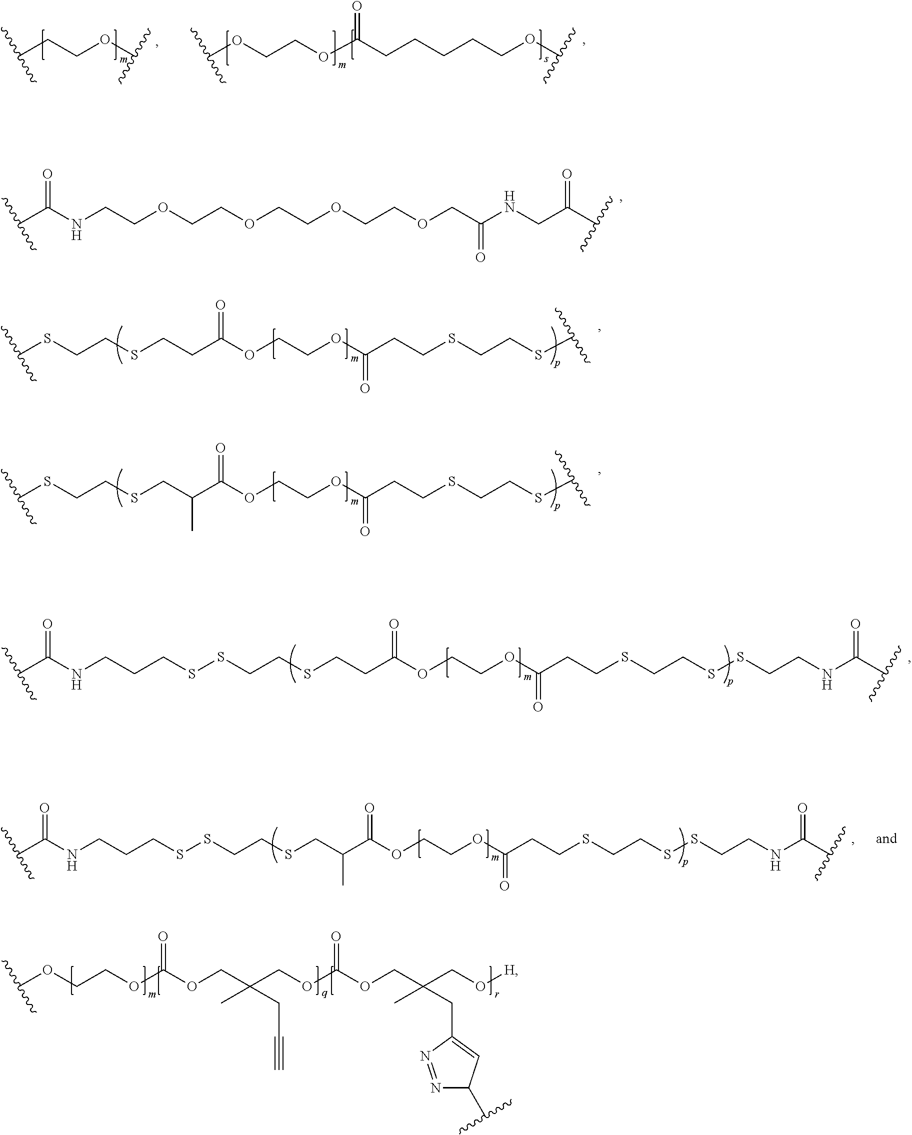

wherein:
m is an integer selected from 1 to 5;
p is an integer selected from 1 to 3;
q is an integer selected from 1 to 5;
r is an integer selected from 1 to 5; and
s is an integer selected from 1 to 5.

In some embodiments, L comprises one or more of PEGDA-DTT, PEGDA-DET, PEGDA-DPT, PEGDA-DBT, PEGDMA-DTT, PEGDMA-DET, PEGDMA-DPT, PEGDMA-DBT, wherein the molecular weight for PEGDA is selected from 258, 575, and 700, and wherein the molecular weight for PEGDMA is selected from 550 and 750. See, for example, Yi et al. Polymer Chem. 7, 5966-5977 (2016), the entire contents of which is hereby incorporated by reference.

Additional examples of linkers can be found in Pawełczyk et al. Int. J. Mol. Sci. 19, 1104-1122 (2018), Osada et al., J. R. Soc. Interface, S325-S339 (2009), Gupta et al., Chem. Sci. 8, 2387-2395 (2017), Wang et al., Macromolecular Bioscience 7, 1187-1198 (2007), Yi et al. Polymer Chem. 7, 5966-5977 (2016), WO2020069353, WO2019118924, WO2019210215, WO2018175922, WO2017053638, WO2017117464, WO2018191548, WO2019169341, WO2019148291, WO2019148293, and WO2019148294, the entire contents of all of which are hereby incorporated by reference.

In some embodiments, n is 1. In some embodiments, n is 2. In some embodiments, n is 3. In some embodiments, n is 4.

In some embodiments, one or more L is further covalently linked to an active agent C2. In some embodiments, C2 is a radical formed from an active agent, and L is covalently linked to C2. In some embodiments, the compound of formula (I) comprises one, two, three, or four C2. In some embodiments, each occurrence of C2 is the same. In some embodiments, the compound of formula (I) includes two or more C2, and all C2s have the same structure. In some embodiments, each L is covalently linked to each C2 at analogous sites. In some embodiments, each L is covalently linked to each C2 at non-analogous sites. In some embodiments, each occurrence of C2 is different. In some embodiments, the compound of formula (I) includes two or more C2, and one or more C2 is of a different structure from one or more other C2. In some embodiments, the compound of formula (I) includes two or more C2, and each C2 is of a different structure from each other.

In some embodiments, C2 is a radical formed from an active agent. In a non-limiting example, the C2 radical is formed from removal of a hydrogen atom from the active agent, and the active agent is covalently linked to an atom and/or group (e.g. covalently linked to L, or covalently linked to atom and/or group R in-R-L when Lis a single bond).

In some embodiments, each L is covalently linked to the same C2 group. For example, the compound of formula (I) includes one C2 molecule, and each L is covalently linked to a different site on the one C2 group. In some embodiments, each L is covalently linked to a different C2 group. In some embodiments, one or more L is covalently linked to the same C2 group, and one or more L is covalently linked to a different C2 group.

In some embodiments, the compound of formula (I) is a dimer (comprises one L covalently linked to a C2). In some embodiments, the compound of formula (I) is a homodimer (C1 and C2 have the same structure). In some embodiments, C2 is a heterodimer (C1 and C2 do not have the same structure).

In some embodiments, C2 comprises one or more of an anti-vascular endothelial growth factor (VEGF) agent, a modulator of the complement cascade, an NSAID, an angiotensin-converting enzyme (ACE) inhibitor, a peroxisome proliferator-activated receptor (PPAR) modulator, a renin inhibitor, a corticosteroid, an antibody, a soluble receptor, a biological molecule, a nuclear receptor protein (e.g. LXR), a Liver X receptor (LXR) agonist, a steroid (e.g. fluocinolone acetonide, dexamethasone), a fatty acid mimic (FAM) molecule, and an agent that modulates autophagy.

In some embodiments, C2 comprises an angiotensin-converting enzyme (ACE) inhibitor. Non limiting examples of angiotensin-converting enzyme (ACE) inhibitors include, but are not limited to: alacepril, alatriopril, altiopril calcium, ancovenin, benazepril, benazepril hydrochloride, benazeprilat, benzazepril, benzoylcaptopril, captopril, captoprilcysteine, captoprilglutathione, ceranapril, ceranopril, ceronapril, cilazapril, cilazaprilat, converstatin, delapril, delaprildiacid, enalapril, enalaprilat, enalkiren, enapril, epicaptopril, foroxymithine, fosfenopril, fosenopril, fosenopril sodium, fosinopril, fosinopril sodium, fosinoprilat, fosinoprilic acid, glycopril, hemorphin-4, idapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spirapril hydrochloride, temocapril, temocapril hydrochloride, teprotide, trandolapril, trandolaprilat, utibapril, zabicipril, zabiciprilat, zofenopril, zofenoprilat, pharmaceutically acceptable salts thereof, and mixtures thereof. In some embodiments, C2 comprises a PPAR modulator. In some embodiments, the PPAR modulator is selected from a PPAR-α modulator, a PPAR-β/o modulator, and a PPAR-γ modulator. In some embodiments, the PPAR modulator is selected from a PPAR agonist, a PPAR antagonist, a PPAR partial agonist, and a PPAR inverse agonist.

In some embodiments, the PPAR modulator is a PPAR-γ modulator. In some embodiments, the PPAR-γ modulator is a full agonist or a partial agonist. In some embodiments, the PPAR-γ modulator is a member of the drug class of thiazolidinediones (TZDs, or glitazones). By way of non-limiting example, the PPAR-γ modulator may be one or more of rosiglitazone (AVANDIA), pioglitazone (ACTOS), troglitazone (REZULIN), netoglitazone, rivoglitazone, ciglitazone, rhodanine. In some embodiments, the PPAR-γ modulator is one or more of irbesartan and telmesartan. In some embodiments, the PPAR-γ modulator is a nonsteroidal anti-inflammatory drugs (NSAID, such as, for example, ibuprofen) and indoles. Known inhibitors include the experimental agent GW-9662. Further examples of PPAR-γ modulators are described in WIPO Publication Nos. WO/1999/063983, WO/2001/000579, Nat Rev Immunol. 2011 Oct. 25;11 (11): 750-61, or agents identified using the methods of WO/2002/068386, the contents of which are hereby incorporated by reference in their entireties.

In some embodiments, the PPAR-γ modulator is a “dual,” or “balanced,” or “pan” PPAR modulator. In some embodiments, the PPAR-γ modulator is a glitazar, which bind two or more PPAR isoforms, e.g., muraglitazar (Pargluva) and tesaglitazar (Galida) and aleglitazar.

In some embodiments, C2 comprises a renin inhibitor. Non limiting examples of renin inhibitors which are useful in the present disclosure include, but are not limited to: aliskiren, ditekiren, enalkiren, remikiren, terlakiren, ciprokiren and zankiren, pharmaceutically acceptable salts thereof, and mixtures thereof.

In some embodiments, C2 comprises a steroid. In some embodiments, a steroid is a compound belonging to or related to the following illustrative families of compounds: corticosteroids, mineralocorticoids, and sex steroids (including, for example, potentially androgenic or estrogenic or anti-andogenic and anti-estrogenic molecules). Included amongst these are, by way of non-limiting example, prednisone, prednisolone, methyl-prednisolone, triamcinolone, fluocinolone, aldosterone, spironolactone, danazol (otherwise known as OPTINA), and others.

In some embodiments, C2 comprises a non-steroidal anti-inflammatory drug (NSAIDs). In some embodiments, C2 is directly conjugated to NSAIDs. In some embodiments, C2 is indirectly conjugated to NSAIDs. In some embodiments, NSAIDs include indomethacin (Indocin), ibuprofen, diclofenac (Voltaren), acemetacin, diclofenac, naproxen, ketorolac, carprofen, etodolac, fentiazac, flurbiprofen, ketoprofen, oxaprozin, pranoprofen, sulindac, suxibuzone, celecoxib (Celebrex®), fenoprofin (Nalfon), ketorolac tromethamine, tolmetin, diflunisal, meclofenamate sodium, mefenamic acid, etoricoxib, meloxicam, nabumetone, piroxicam, or derivatives thereof. In some embodiments, the NSAID is indomethacin (Indocin). In some embodiments, the NSAID is ibuprofen. In some embodiments, the NSAID is diclofenac (Voltaren). In some embodiments, the NSAID is acemetacin. In some embodiments, the NSAID is diclofenac. In some embodiments, the NSAID is naproxen. In some embodiments, the NSAID is ketorolac. In some embodiments, the NSAID is carprofen. In some embodiments, the NSAID is etodolac. In some embodiments, the NSAID is fentiazac. In some embodiments, the NSAID is flurbiprofen. In some embodiments, the NSAID is ketoprofen. In some embodiments, the NSAID is oxaprozin. In some embodiments, the NSAID is pranoprofen. In some embodiments, the NSAID is sulindac. In some embodiments, the NSAID is suxibuzone. In some embodiments, the NSAID is celecoxib (Celebrex®). In some embodiments, the NSAID is fenoprofin (Nalfon). In some embodiments, the NSAID is ketorolac tromethamine. In some embodiments, the NSAID is tolmetin. In some embodiments, the NSAID is diflunisal. In some embodiments, the NSAID is meclofenamate sodium. In some embodiments, the NSAID is mefenamic acid. In some embodiments, the NSAID is etoricoxib. In some embodiments, the NSAID is meloxicam. In some embodiments, the NSAID is nabumetone. In some embodiments, the NSAID is piroxicam. In some embodiments C2 comprises acetylsalicylic acid (aspirin). In some embodiments, the NSAID is a FAM. In some embodiments, the NSAID sits within the beta-barrel domain. In some embodiments, the NSAID thereof interacts with about 3, about 4, about 5, about 6, about 7, or about 8 sites within the pocket of the beta barrel domain (e.g. the NSAID forms hydrogen bonds with amino acids of the beta barrel domain).

In some embodiments, C2 comprises an agent that modulates autophagy, microautophagy, mitophagy or other forms of autophagy. In some embodiments, the candidate drug and/or compound is one or more of sirolimus, tacrolimis, rapamycin, everolimus, bafilomycin, chloroquine, hydroxychloroquine, spautin-1, metformin, perifosine, resveratrol, trichostatin, valproic acide, Z-VAD-FMK, or others known to those in the art. Without wishing to be bound by theory, agent that modulates autophagy, microautophagy, mitophagy or other forms of autophagy may alter the recycling of intra-cellular components, for example, but not limited to, cellular organelles, mitochondria, endoplasmic reticulum, lipid or others. Without further wishing to be bound by theory, this agent may or may not act through microtubule-associated protein 1A/1B-light chain 3 (LC3). In embodiments, C2 is or comprises an agent that targets mitochondrial stability and/or function. In embodiments, C2 comprises an agent that targets phospholipid cardiolipin. In embodiments, the agent comprises Elamipretide, SBT-272, or SBT-550.

In some embodiments, C2 comprises an anti-angiogenic agent. In some embodiments, the anti-angiogenic agent is selected from Axitinib (Inlyta), Bevacizumab (Avastin), Cabozantinib (Cometriq), Everolimus (Afinitor, Zortress), Lenalidomide (Revlimid), Pazopanib (Votrient), Ramucirumab (Cyramza), Regorafenib (Stivarga), Sorafenib (Nexavar), Sunitinib (Sutent), Thalidomide (Synovir, Thalomid), Vandetanib (Caprelsa), Ziv-aflibercept (Zaltrap), Aflibercept (Eylea), Ranibizumab (Lucentis), Brolucizumab, Abicipar (a DARPin), KSI-301 Antibody Biopolymer Conjugate (Kodiak sciences) (e.g. for extended release in ophthalmology), OPT-302 (Opthea) (VEGF-C/D trap molecule), Faricimab (anti-VEGF & anti-angiopoietin-2), DE-122, and anti-endoglin (Tracon & Santen) (e.g. for anti-angiogenesis in ophthalmology).

In some embodiments, C2 comprises an anti-VEGF agent. Non-limiting examples of anti-VEGF agents include ranibizumab, bevacizumab, aflibercept, KH902 VEGF receptor-Fc, fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-, hydroxyisovalerylshikonin, ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, G6-31 antibody, a fusion antibody and an antibody that binds to an epitope of VEGF. Additional non-limiting examples of anti-VEGF agents useful in the present methods include a substance that specifically binds to one or more of a human vascular endothelial growth factor-A (VEGF-A), human vascular endothelial growth factor-B (VEGF-B), human vascular endothelial growth factor-C(VEGF-C), human vascular endothelial growth factor-D (VEGF-D) and human vascular endothelial growth, factor-E (VEGF-E), and an antibody that binds, to an epitope of VEGF.

In one embodiment, the anti-VEGF agent is the antibody ranibizumab or a pharmaceutically acceptable salt thereof. Ranibizumab is commercially available under the trademark LUCENTIS. In another embodiment, the anti-VEGF agent is the antibody bevacizumab or a pharmaceutically acceptable salt thereof. Bevacizumab is commercially available under the trademark AVASTIN. In another embodiment, the anti-VEGF agent is the antibody brolucizumab or a pharmaceutically acceptable salt thereof. Brolucizumab is commercially available under the trademark BEOVU. In another embodiment, the anti-VEGF agent is aflibercept or a pharmaceutically acceptable salt thereof. Aflibercept is commercially available under the trademark EYLEA. In one embodiment, the anti-VEGF agent is pegaptanib or a pharmaceutically acceptable salt thereof. Pegaptinib is commercially available under the trademark MACUGEN. In another embodiment, the anti-VEGF agent is an antibody or an antibody fragment that binds to an epitope of VEGF, such as an epitope of VEGF-A, VEGF-B, VEGF-C, VEGF-D, or VEGF-E. In some embodiments, the VEGF antagonist binds to an epitope of VEGF such that binding of VEGF and VEGFR are inhibited. In one embodiment, the epitope encompasses a component of the three dimensional structure of VEGF that is displayed, such that the epitope is exposed on the surface of the folded VEGF molecule. In one embodiment, the epitope is a linear amino acid sequence from VEGF.

In some embodiments, C2 comprises a low molecular weight anti-VEGF agent. In some embodiments, the low molecular weight anti-VEGF agent is selected from thalidomide (Synovir, Thalomide), an mTOR inhibitor (e.g. temsirolimus, everolimus (Afinitor, Zortress), tacrolimus or rapalogues (e.g. rapamycin), or corticosteroids (dexamethasone, Fluocinolone acetonide, triamcinolone), aflibercept, and a tyrosine kinase inhibitor.

In some embodiments, C2 comprises a tyrosine kinase inhibitor. In some embodiments, the tyrosine kinase inhibitor is selected from Vatalanib (PTK787), lapatinib, Sunitinib (Sutent), Sorafenib (Nexavar), Ramucirumab (Cyramza), Nintedanib, Lenvatinib, Anlotinib, Fruquintinib, Apatinib, Pazopanib (Votrient), Axitinib (Inlyta), Cabozantinib (Cometriq), Vandetanib (Caprelsa), and Regorafenib (Stivarga).

In some embodiments, C2 comprises a polyphenol. Non-limiting examples of polyphenols include catechin, resveratrol, hesperidin, chlorogenic acid, flavonols, isoflavones, tannins, quercetin, and anthocyanins. In some embodiments, C2 comprises resveratrol.

In some embodiments, C2 comprises a biological molecule. In some embodiments, the biological molecule is selected from an antibody, an antibody fragment, a soluble receptor (e.g. solVEGFR1 [sol flt-1] or solVEGFR2), a DARPin, a microRNA (miRNA), Bevacizumab (Avastin), Ziv-aflibercept (Zaltrap), Aflibercept (Eylea), Ranibizumab (Lucentis), Brolucizumab (Beovu), Abicipar (AbbVie, a DARPin), KSI-301 Antibody Biopolymer Conjugate (Kodiak sciences) (e.g. for extended release in ophthalmology), OPT-302 (Opthea) (VEGF-C/D trap molecule), Faricimab (anti-VEGF & anti-angiopoietin-2), and DE-122, and anti-endoglin (Tracon & Santen) (e.g. for anti-angiogenesis in ophthalmology).

In some embodiments, C2 comprises a soluble receptor. Non-limiting examples of soluble receptors include receptor solVEGFR1 [sol flt-1] and solVEGFR2.

In some embodiments, C2 comprises bindarit or an analogue thereof. In some embodiments, C2 is a radical formed from bindarit or the analogue thereof, and C2 is covalently linked to L.

In some embodiments, bindarit or the analogue thereof comprises a group selected from:

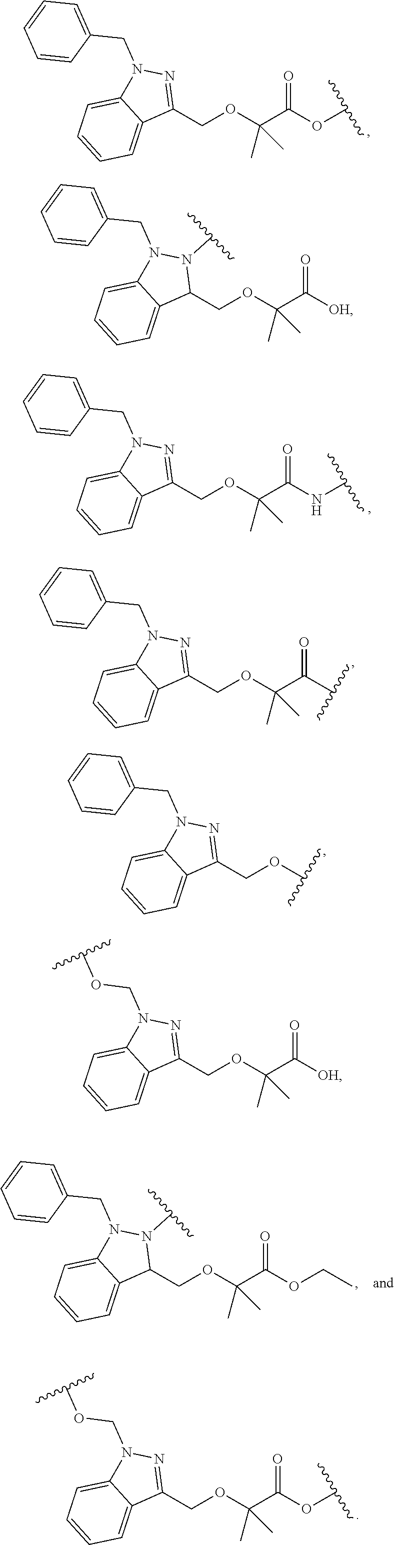

In some embodiments, C2 comprises a bindarit ethyl ester (a.k.a. ethyl 2-((1-benzl-1H-indazol-3-yl)methoxy)-2-methylpropanoate) or an analogue thereof. In some embodiments, C2 is a radical formed from bindarit ethyl ester or an analogue thereof, and C2 is covalently linked to L. In some embodiments, the compound of formula (I) is a compound of formula (If), formula (Ig), formula (Ih), formula (Ii), formula (Ij), formula (Ik), formula (Im), formula (In), or a pharmaceutically acceptable salt thereof:

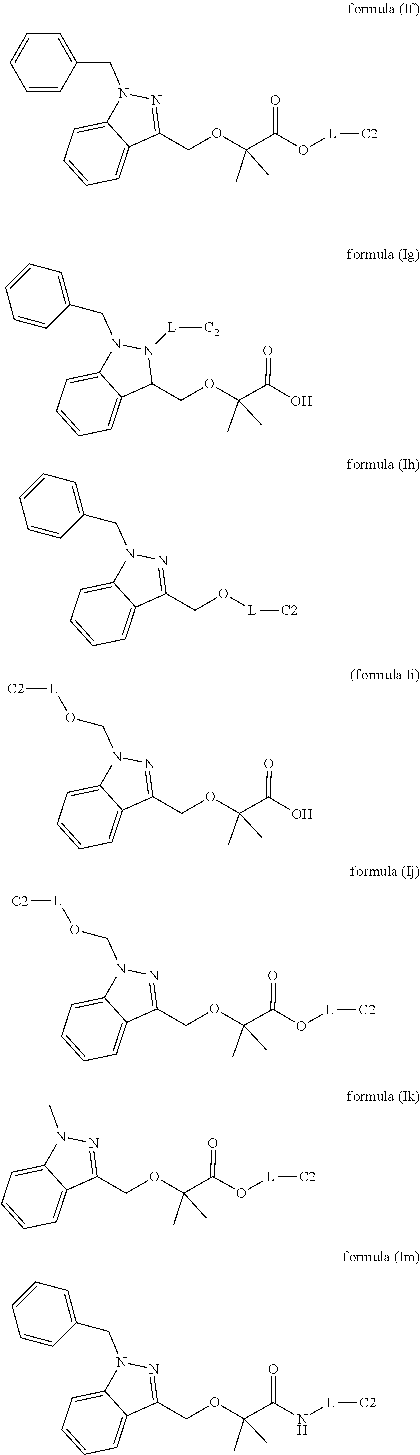

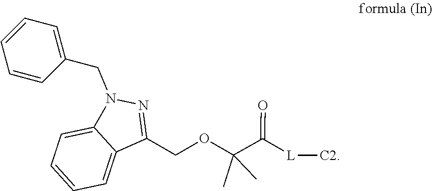

In some embodiments, C2 comprises one or more of bindarit or an analogue thereof, an anti-angiogenic agent, optionally a biological anti-angiogenic agent, (e.g. angiopoietin and placental growth factor), an anti-VEGF agent, optionally a low molecular weight anti-VEGF agent.

In some embodiments, C2 comprises one or more of PTK787, aflibercept, Brolucizumab, Triamcinolone, Dexamethasone, OPT-302, and DE-122.

In some embodiments, C2 comprises one or more nutraceutical agents. Non-limiting examples of nutraceutical agents include, resveratrol, taurine, carnosine, quercetin, curcumin, glycyrrhizin, naringin, folate, glutathione, n-acetylcysteine, alpha lipoic acid, quercetin, zinc, vitamin a, vitamin c, vitamin d, vitamin k2, vitamin b, vitamin b6, vitamin b9, vitamin b12, vitamin e, magnesium, bromelain, cannabidiolic acid, cannabigerolic acid, cannabidiol, cannabigerol, cannabinol, nigeIIa sativa/thymoquinone, selenium, curcumin, piperine, astaxanthin, and/or sulforaphane. In some embodiments, the nutraceutical agent is resveratrol. In some embodiments, the nutraceutical agent is taurine. In some embodiments, the nutraceutical agent is carnosine.

In some embodiments, in formula (I), C2 comprises one or more nutraceutical agents (e.g. resveratrol taurine, or carnosine) and L comprises one or more esters. In some embodiments, L is a cleavable linker. In some embodiments, L is a non-cleavable linker.

In some embodiments, C2 comprises one or more peptides. In some embodiments, the peptide comprises about 1, about 2, about 3, about 4, about 5, or about 5 or more amino acids. In some embodiments, C2 comprises one or more monopeptides, dipeptides, tripeptides, and/or tetrapeptides. In a non-limiting embodiment, a monopeptide comprises one amino acid. In a non-limiting embodiment, a dipeptide comprises two amino acids, which may each be the same or different. In a non-limiting embodiment, a tripeptide comprises three amino acids, which may each be the same or different. In a non-limiting embodiment, a tetrapeptide comprises four amino acids, which may each be the same or different. Non-limiting examples of a monopeptides include non-proteinogenic amino acids such as taurine, ornithine, citrulline, arginosuccinate, homoserine, homocysteine, and cystathionine. Non-limiting examples of dipeptides include aspartame and glutamine-taurine. Non-limiting examples of tripeptides and tetrapeptides include combinations of three (tripeptides) or four (tetrapeptides) amino acids selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and proteinogenic amino acids such as taurine, ornithine, citrulline, arginosuccinate, homoserine, homocysteine, cystathionine.

In some embodiments, in formula (I), C2 comprises one or more peptides (e.g. taurine, carnosine, or aspartame) and L comprises a tripeptide comprising and/or consisting of arginine, glycine, and aspartic acid (RGD).

In some embodiments, C2 comprises one or more proteins. Non-limiting examples of proteins include an anti-VEGF antibody (e.g. Ranibizumab) and a soluble receptor (e.g. solVEGFR1 [sol flt-1] or solVEGFR2).

In some embodiments, in formula (I), C2 comprises one or more proteins (e.g. an anti-VEGF antibody or a soluble receptor) and L comprises

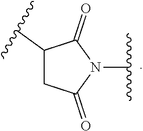 [SMILES: *C1CC(=O)N(*)C1=O]

In some embodiments, L iis a cleavable linker. In some embodiments, L is a non-cleavable linker.

In some embodiments, in formula (If), L is a single bond. In some embodiments, in formula (If), C2 is selected from resveratrol, carnosine, and taurine. In some embodiments, in formula (If), L is a single bond and C2 is selected from resveratrol, carnosine, and taurine. In some embodiments, in formula (If), C2 is resveratrol. In some embodiments, in formula (If), C2 is carnosine. In some embodiments, in formula (If), C2 is taurine. In some embodiments, in formula (If), L is a single bond and C2 comprises resveratrol. In some embodiments, in formula (If), L is a single bond and C2 is carnosine. In some embodiments, in formula (If), L is a single bond and C2 comprises taurine. In some embodiments, in formula (If), C2 is a radical of resveratrol, taurine, or carnosine. In some embodiments, in formula (If), C2 is a radical of resveratrol. In some embodiments, in formula (If), C2 is a radical of taurine. In some embodiments, in formula (If), C2 is a radical of carnosine. In some embodiments, in formula (If), L is a single bond and C2 is a radical of resveratrol, taurine, or carnosine. In some embodiments, in formula (If), L is a single bond and C2 is a radical of resveratrol. In some embodiments, in formula (If), L is a single bond and C2 is a radical of taurine. In some embodiments, in formula (If), L is a single bond and C2 is a radical of carnosine.

In some embodiments, in formula (Ig), L is a single bond. In some embodiments, in formula (Ig), C2 is selected from resveratrol, carnosine, and taurine. In some embodiments, in formula (Ig), L is a single bond and C2 is selected from resveratrol, carnosine, and taurine. In some embodiments, in formula (Ig), C2 comprises resveratrol. In some embodiments, in formula (Ig), C2 is carnosine. In some embodiments, in formula (Ig), C2 comprises taurine. In some embodiments, in formula (Ig), L is a single bond and C2 comprises resveratrol. In some embodiments, in formula (Ig), L is a single bond and C2 comprises carnosine. In some embodiments, in formula (Ig), L is a single bond and C2 comprises taurine. In some embodiments, in formula (Ig), C2 is a radical of resveratrol, taurine, or carnosine. In some embodiments, in formula (Ig), C2 is a radical of resveratrol. In some embodiments, in formula (Ig), C2 is a radical of taurine. In some embodiments, in formula (Ig), C2 is a radical of carnosine. In some embodiments, in formula (Ig), L is a single bond and C2 is a radical of resveratrol, taurine, or carnosine. In some embodiments, in formula (Ig), L is a single bond and C2 is a radical of resveratrol. In some embodiments, in formula (Ig), L is a single bond and C2 is a radical of taurine. In some embodiments, in formula (Ig), L is a single bond and C2 is a radical of carnosine.

In some embodiments, in formula (Ih), L is a single bond. In some embodiments, in formula (Ih), C2 is selected from resveratrol, carnosine, and taurine. In some embodiments, in formula (Ih), L is a single bond and C2 is selected from resveratrol, carnosine, and taurine. In some embodiments, in formula (Ih), C2 comprises resveratrol. In some embodiments, in formula (Ih), C2 comprises carnosine. In some embodiments, in formula (Ih), C2 comprises taurine. In some embodiments, in formula (Ih), L is a single bond and C2 comprises resveratrol. In some embodiments, in formula (Ih), L is a single bond and C2 comprises carnosine. In some embodiments, in formula (Ih), L is a single bond and C2 comprises taurine. In some embodiments, in formula (Ih), C2 is a radical of resveratrol, taurine, or carnosine. In some embodiments, in formula (Ih), C2 is a radical of resveratrol. In some embodiments, in formula (Ih), C2 is a radical of taurine. In some embodiments, in formula (Ih), C2 is a radical of carnosine. In some embodiments, in formula (Ih), L is a single bond and C2 is a radical of resveratrol, taurine, or carnosine. In some embodiments, in formula (Ih), L is a single bond and C2 is a radical of resveratrol. In some embodiments, in formula (Ih), L is a single bond and C2 is a radical of taurine. In some embodiments, in formula (Ih), L is a single bond and C2 is a radical of carnosine.

In some embodiments, in formula (Ii), L is a single bond. In some embodiments, in formula (Ii), C2 is selected from resveratrol, carnosine, and taurine. In some embodiments, in formula (Ii), L is a single bond and C2 is selected from resveratrol, carnosine, and taurine. In some embodiments, in formula (Ii), C2 comprises resveratrol. In some embodiments, in formula (Ii), C2 comprises carnosine. In some embodiments, in formula (Ii), C2 comprises taurine. In some embodiments, in formula (Ii), L is a single bond and C2 comprises resveratrol. In some embodiments, in formula (Ii), L is a single bond and C2 is carnosine. In some embodiments, in formula (Ii), L is a single bond and C2 comprises taurine. In some embodiments, in formula (Ii), C2 is a radical of resveratrol, taurine, or carnosine. In some embodiments, in formula (Ii), C2 is a radical of resveratrol. In some embodiments, in formula (Ii), C2 is a radical of taurine. In some embodiments, in formula (Ii), C2 is a radical of carnosine. In some embodiments, in formula (Ii), L is a single bond and C2 is a radical of resveratrol, taurine, or carnosine. In some embodiments, in formula (Ii), L is a single bond and C2 is a radical of resveratrol. In some embodiments, in formula (Ii), L is a single bond and C2 is a radical of taurine. In some embodiments, in formula (Ii), L is a single bond and C2 is a radical of carnosine.

In some embodiments, in formula (Ij), L is a single bond. In some embodiments, in formula (Ij), C2 is selected from resveratrol, carnosine, and taurine. In some embodiments, in formula (Ij), L is a single bond and C2 is selected from resveratrol, carnosine, and taurine. In some embodiments, in formula (Ij), C2 comprises resveratrol. In some embodiments, in formula (Ij), C2 comprises carnosine. In some embodiments, in formula (Ij), C2 comprises taurine. In some embodiments, in formula (Ij), L is a single bond and C2 comprises resveratrol. In some embodiments, in formula (Ij), L is a single bond and C2 comprises carnosine. In some embodiments, in formula (Ij), L is a single bond and C2 comprises taurine. In some embodiments, in formula (Ij), C2 is a radical of resveratrol, taurine, or carnosine. In some embodiments, in formula (Ij), C2 is a radical of resveratrol. In some embodiments, in formula (Ij), C2 is a radical of taurine. In some embodiments, in formula (Ij), C2 is a radical of carnosine. In some embodiments, in formula (Ij), L is a single bond and C2 is a radical of resveratrol, taurine, or carnosine. In some embodiments, in formula (Ij), L is a single bond and C2 is a radical of resveratrol. In some embodiments, in formula (Ij), L is a single bond and C2 is a radical of taurine. In some embodiments, in formula (Ij), L is a single bond and C2 is a radical of carnosine.

In some embodiments, in formula (Ik), L is a single bond. In some embodiments, in formula (Ik), C2 is selected from resveratrol, carnosine, and taurine. In some embodiments, in formula (Ik), L is a single bond and C2 is selected from resveratrol, carnosine, and taurine. In some embodiments, in formula (Ik), C2 comprises resveratrol. In some embodiments, in formula (Ik), C2 comprises carnosine. In some embodiments, in formula (Ik), C2 comprises taurine. In some embodiments, in formula (Ik), L is a single bond and C2 comprises resveratrol. In some embodiments, in formula (Ik), L is a single bond and C2 comprises carnosine. In some embodiments, in formula (Ik), L is a single bond and C2 comprises taurine. In some embodiments, in formula (Ik), C2 is a radical of resveratrol, taurine, or carnosine. In some embodiments, in formula (Ik), C2 is a radical of resveratrol. In some embodiments, in formula (Ik), C2 is a radical of taurine. In some embodiments, in formula (Ik), C2 is a radical of carnosine. In some embodiments, in formula (Ik), L is a single bond and C2 is a radical of resveratrol, taurine, or carnosine. In some embodiments, in formula (Ik), L is a single bond and C2 is a radical of resveratrol. In some embodiments, in formula (Ik), L is a single bond and C2 is a radical of taurine. In some embodiments, in formula (Ik), L is a single bond and C2 is a radical of carnosine.

In some embodiments, in formula (Im), L is a single bond. In some embodiments, in formula (Im), C2 is selected from resveratrol, carnosine, and taurine. In some embodiments, in formula (Im), L is a single bond and C2 is selected from resveratrol, carnosine, and taurine. In some embodiments, in formula (Im), C2 comprises resveratrol. In some embodiments, in formula (Im), C2 comprises carnosine. In some embodiments, in formula (Im), C2 comprises taurine. In some embodiments, in formula (Im), L is a single bond and C2 comprises resveratrol. In some embodiments, in formula (Im), L is a single bond and C2 comprises carnosine. In some embodiments, in formula (Im), L is a single bond and C2 comprises taurine. In some embodiments, in formula (Im), C2 is a radical of resveratrol, taurine, or carnosine. In some embodiments, in formula (Im), C2 is a radical of resveratrol. In some embodiments, in formula (Im), C2 is a radical of taurine. In some embodiments, in formula (Im), C2 is a radical of carnosine. In some embodiments, in formula (Im), L is a single bond and C2 is a radical of resveratrol, taurine, or carnosine. In some embodiments, in formula (Im), L is a single bond and C2 is a radical of resveratrol. In some embodiments, in formula (Im), L is a single bond and C2 is a radical of taurine. In some embodiments, in formula (Im), L is a single bond and C2 is a radical of carnosine.

In some embodiments, in formula (In), L is a single bond. In some embodiments, in formula (In), C2 is selected from resveratrol, carnosine, and taurine. In some embodiments, in formula (In), L is a single bond and C2 is selected from resveratrol, carnosine, and taurine. In some embodiments, in formula (In), C2 comprises resveratrol. In some embodiments, in formula (In), C2 comprises carnosine. In some embodiments, in formula (In), C2 is taurine. In some embodiments, in formula (In), L is a single bond and C2 comprises resveratrol. In some embodiments, in formula (In), L is a single bond and C2 comprises carnosine. In some embodiments, in formula (In), L is a single bond and C2 i comprises taurine. In some embodiments, in formula (In), C2 is a radical of resveratrol, taurine, or carnosine. In some embodiments, in formula (In), C2 is a radical of resveratrol. In some embodiments, in formula (In), C2 is a radical of taurine. In some embodiments, in formula (In), C2 is a radical of carnosine. In some embodiments, in formula (In), L is a single bond and C2 is a radical of resveratrol, taurine, or carnosine. In some embodiments, in formula (In), L is a single bond and C2 is a radical of resveratrol. In some embodiments, in formula (In), L is a single bond and C2 is a radical of taurine. In some embodiments, in formula (In), L is a single bond and C2 is a radical of carnosine.

In some embodiments, the compound of formula (I) is a macrocycle. In a non-limiting example, the compound of formula (I) comprises one L, and the L is covalently linked to C1 at two sites on C1 to form a macrocycle. In another non-limiting example, the compound of formula (I) comprises two L, and both Ls are covalently linked to the same C2 group at different sites of the same C2 group to form a macrocycle.

In some embodiments, the compound of formula (I) is a compound of formula (IIIa):

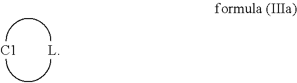

In some embodiments, the compound of formula (I) is a compound of formula (Illb):

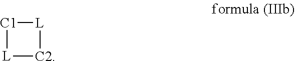

In some embodiments, the compound of formula (I) is of any one of formulas 1001 to 1005, or a pharmaceutically acceptable salt thereof:

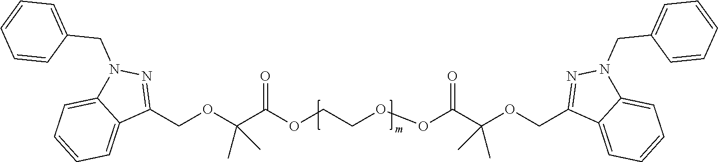

| Compound No. | m |
|---|---|
| 1001 | 1 |
| 1002 | 2 |
| 1003 | 3 |
| 1004 | 4 |
| 1005 | 5 |

In some embodiments, the compound is a compound of formula (I), or a pharmaceutically acceptable salt thereof, wherein:
C1 is selected from bindarit or an analogue thereof and bindarit ethyl ester or an analogue thereof;
L comprises one or more of a bond, —(CH₂CH₂O)—, —C(O), —(CH₂)—, —O—, —C(O)O—, —OC(O)—, —C(O)NR^{a}—, —NR^{a}C(O)—, —S—, —S—S—, —SCH₂CH₂S—, —NR^{a}—, —S(O)—, —S(O)₂—, —CR^{a}₂—, —C(O)S—, —SC(O)—, —C(O)NR^{a}SO₂—, —SO₂NR^{a} C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; L is further covalently linked to C2;
C2 is selected from PTK787, aflibercept, Brolucizumab, Triamcinolone, Dexamethasone, OPT-302, and DE-122; and n is 1.

In some embodiments, the compound is a compound of formula (I), or a pharmaceutically acceptable salt thereof, wherein:
C1 is selected from bindarit or an analogue thereof and bindarit ethyl ester or an analogue thereof; L comprises one or more of a bond, —(CH₂CH₂O)—, —C(O), —(CH₂)—, —O—, —C(O)O—, —OC(O)—, —C(O)NR^{a}—, —NR^{a}C(O)—, —S—, —S—S—, —SCH₂CH₂S—, —NR^{a}—, —S(O)—, —S(O)₂—, —CR^{a}₂—, —C(O)S—, —SC(O)—, —C(O)NR^{a}SO₂—, —SO₂NR^{a}C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
L is further covalently linked to C2;
C2 is an NSAID selected from indomethacin (Indocin), ibuprofen, diclofenac (Voltaren), acemetacin, diclofenac, naproxen, ketorolac, carprofen, etodolac, fentiazac, flurbiprofen, ketoprofen, oxaprozin, pranoprofen, sulindac, suxibuzone, celecoxib (Celebrex®), fenoprofin (Nalfon), ketorolac tromethamine, tolmetin, diflunisal, meclofenamate sodium, mefenamic acid, etoricoxib, meloxicam, nabumetone, piroxicam; and n is 1. In some embodiments, C2 is indomethacin. In some embodiments, L is or comprises an ester (e.g. —C(O)O)—).

In some embodiments, the compound is a compound of formula (I) wherein:
C1 is selected from bindarit or an analogue thereof and bindarit ethyl ester or an analogue thereof;
L comprises one or more of a PEG-based linker, a polyester, polypropylene, polyolefin, polyurethane, alhydrogel, vitamin K, caffeine, menadione, benzoic acid, cholesterol, PVP, β-cyclodextrin,

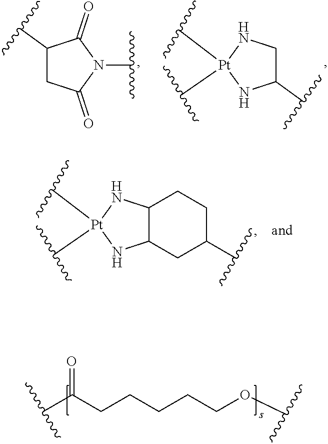

wherein s is 1 to 5;
wherein the PEG-based linker comprises one or more of an amine reactive PEG-based linker (e.g. Bis-PEG-acid, Bis-PEG-NHS, Boc-PEG, Fmoc-PEG, PEG Acid, PEG Aldehyde, PEG NHS ester, PEG Phosphonate, PEG PFP ester, PEG Tosylate), a biotinylated PEG-based linker (e.g. PEG-biotin), a branched PEG-based linker; a reactive carbonyl PEG-based linker (e.g. aminooxy-PEG), a carboxyl and/or active ester reactive PEG-based linker (e.g. amine PEG), a click-reagent PEG-based linker (e.g. alkyne PEG, azide-PEG); a copper-free click chemistry PEG-based linker (e.g. DBCO-PEG, BCN-PEG); a Cu-catalyzed click chemistry PEG-based linker (e.g. propargyl-PEG); a fluorescent PEG labeling and homobifunctional PEG-based linker (e.g. bis-PEG-acid, bis-PEG-NHS, bis-PEG-PFP, bis-propargyl-PEG, amine-PEG-amine, azido-PEG-azide, bromo-PEG-bromide), a lipid PEG, a metal surface binding and thiol reactive PEG-based linker (e.g. Thiol PEG, Bromo-PEG, Mal PEG), PEG-pAsp, PEG-pGlu, PEG-b-PMPMC, and PEG-PGLA;
L is further covalently linked to C2;
C2 is selected from PTK787, aflibercept, Brolucizumab, Triamcinolone, Dexamethasone, OPT-302, and DE-122; and
n is 1.

In some embodiments, the compound is a compound of formula (I), or a pharmaceutically acceptable salt thereof, wherein:
C1 is selected from bindarit or an analogue thereof and bindarit ethyl ester or an analogue thereof;
L comprises one or more of a group selected from:

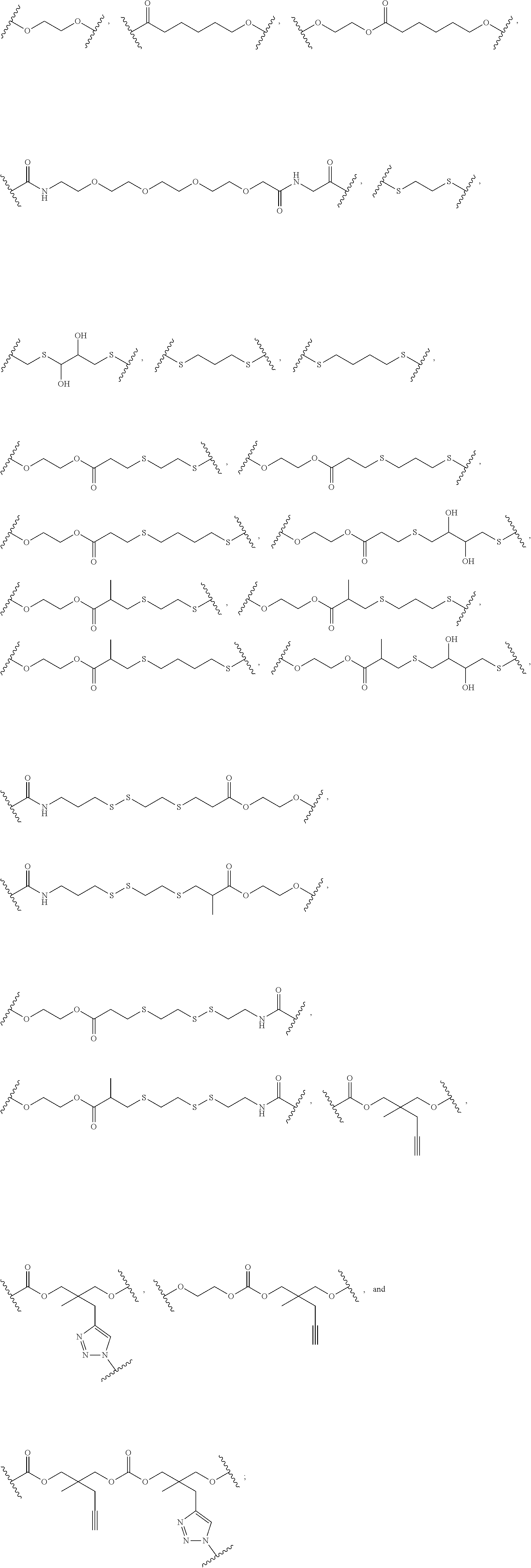

C2 is selected from PTK787, aflibercept, Brolucizumab, Triamcinolone, Dexamethasone, OPT-302, and DE-122; and
n is 1.

In some embodiments, the compound is a compound of formula (I), or a pharmaceutically acceptable salt thereof, wherein:
C1 is selected from bindarit or an analogue thereof and bindarit ethyl ester or an analogue thereof;
L is selected from:

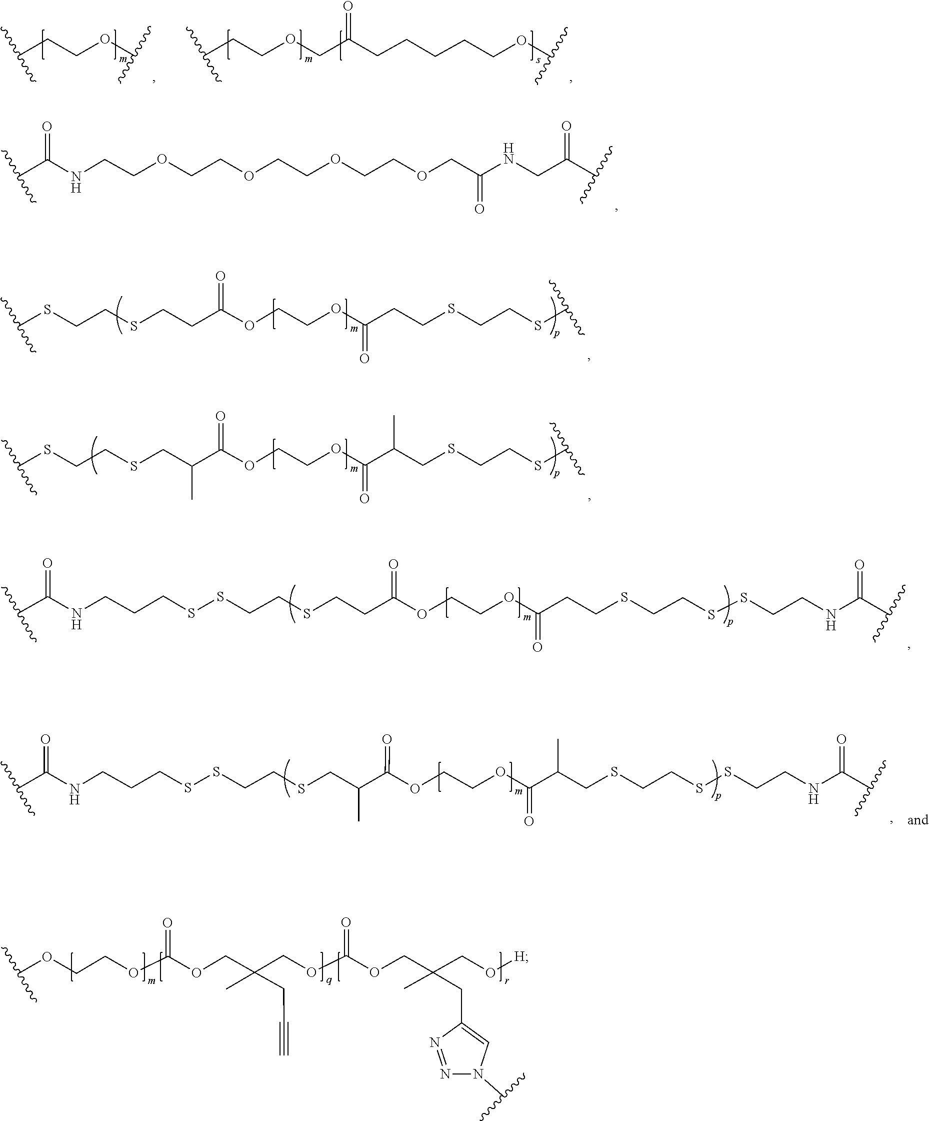

L is further covalently linked to C2;
C2 is selected from PTK787, aflibercept, Brolucizumab, Triamcinolone, Dexamethasone, OPT-302, and DE-122; and
n is 1.

In some embodiments, the compound is a compound of formula (I), or a pharmaceutically acceptable salt thereof, wherein:
C1 is selected from

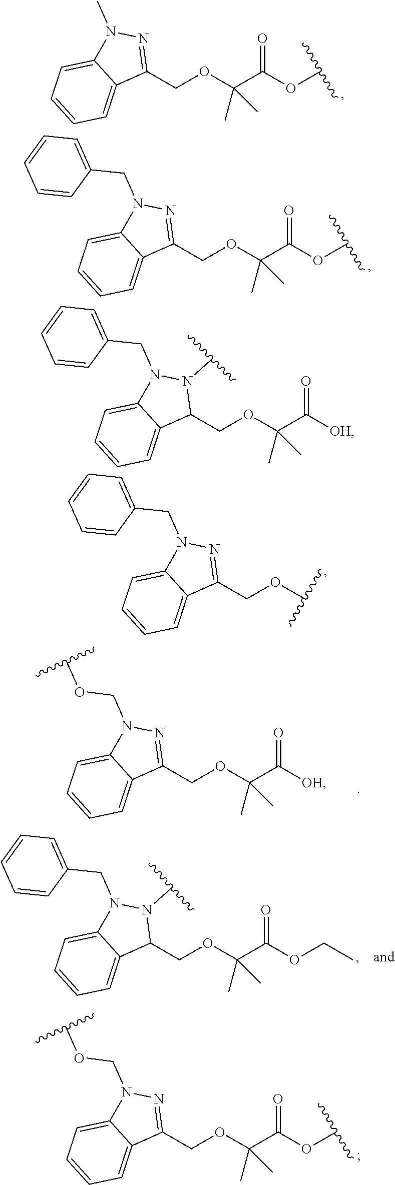

L is selected from:

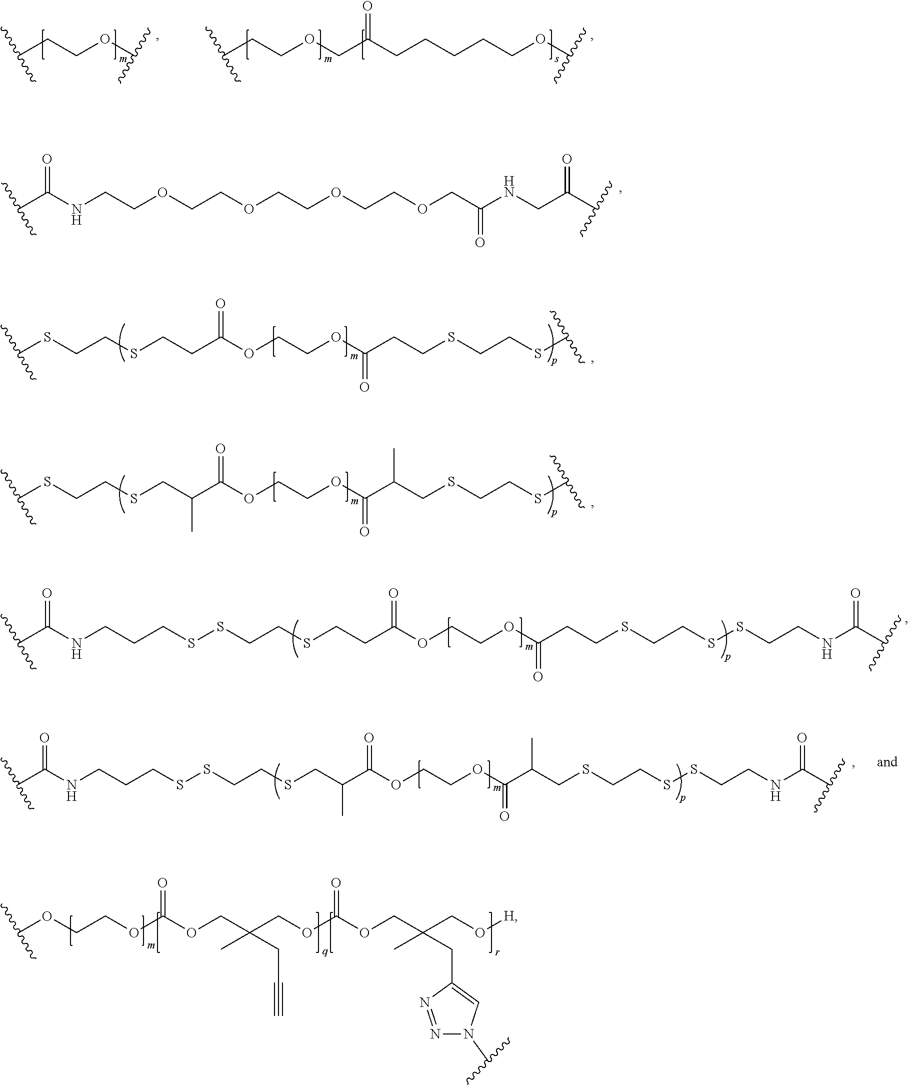

wherein:
   m is an integer selected from 1 to 5;
   p is an integer selected from 1 to 3;
   q is an integer selected from 1 to 5;
   r is an integer selected from 1 to 5; and
   s is an integer selected from 1 to 5;
L is further covalently linked to C2;
C2 is selected from PTK787, aflibercept, Brolucizumab, Triamcinolone, Dexamethasone, OPT-302, and DE-122; and
n is 1.

In some embodiments, the compound of formula (I) is a compound of formula (ila), or a pharmaceutically acceptable salt thereof:

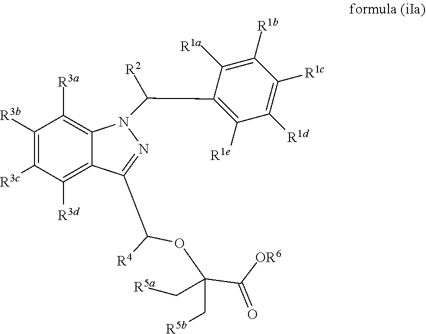

wherein in formula (ila):
one R^{1a}, R^{1b}, R^{1c}, R^{1d}, R^{1e}, R², R^{3a}, R^{3b}, R^{3c}, R^{3d}, R⁴, R^{5a}, R^{5b}, and R⁶ represents -L-C2, and the remaining R^{1a}, R^{1b}, R^{1c}, R^{1d}, R^{1e}, R², R^{3a}, R^{3b}, R^{3c}, R^{3d}, R⁴, R^{5a}, R^{5b}, and R⁶ are independently selected at each occurrence from hydrogen, deuterium, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)R^{a} (where t is 1 or 2), —S(O)OR^{a} (where t is 1 or 2), —S(O)ₜN(R^{a})₂ (where t is 1 or 2), and PO₃(R^{a})₂;
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.
L is selected from:

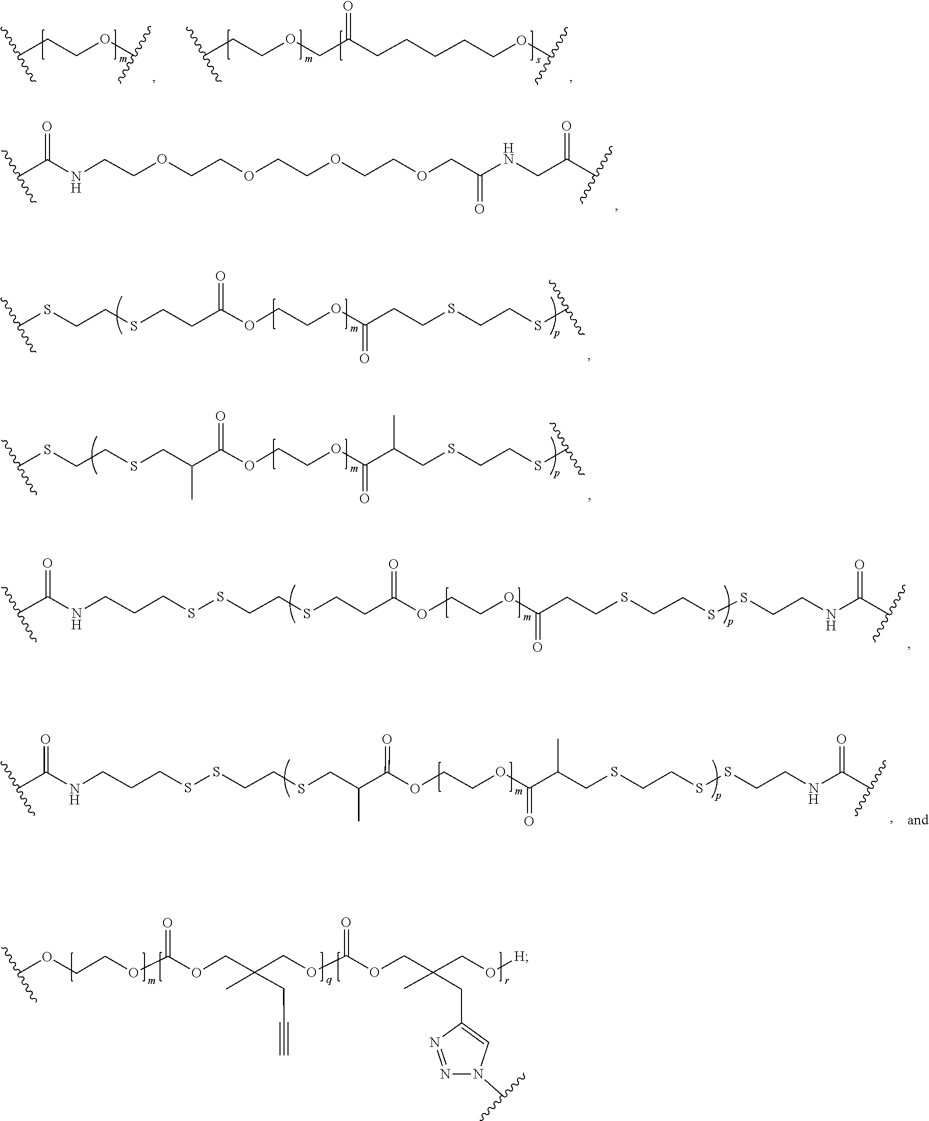

wherein:
   m is an integer selected from 1 to 5;
   p is an integer selected from 1 to 3;
   q is an integer selected from 1 to 5;
   r is an integer selected from 1 to 5; and
   s is an integer selected from 1 to 5
   wherein L is further covalently linked to C2; and
C2 is selected from PTK787, aflibercept, Brolucizumab, Triamcinolone, Dexamethasone, OPT-302, and DE-122.

In some embodiments, the compound of formula (I) is a compound of formula (iIb), or a pharmaceutically acceptable salt thereof:

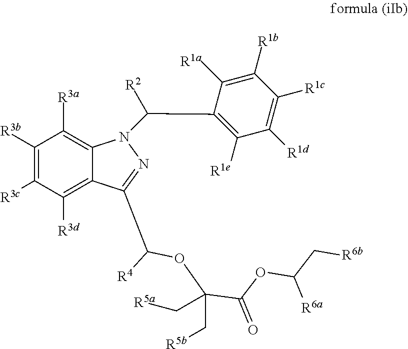

wherein in formula (iIb):
one R^{1a}, R^{1b}, R^{1c}, R^{1d}, R^{1e}, R², R^{3a}, R^{3b}, R^{3c}, R^{3d}, R⁴, R^{5a}, R^{5b}, Roa, and R^{6b} represents -L-C2, and the remaining R^{1a}, R^{1b}, R^{1c}, R^{1d}, R^{1e}, R², R^{3a}, R^{3b}, R^{3c}, R^{3d}, R⁴, R^{5a}, R^{5b}, R^{6a}, and Rob are independently selected at each occurrence from hydrogen, deuterium, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
L is selected from:

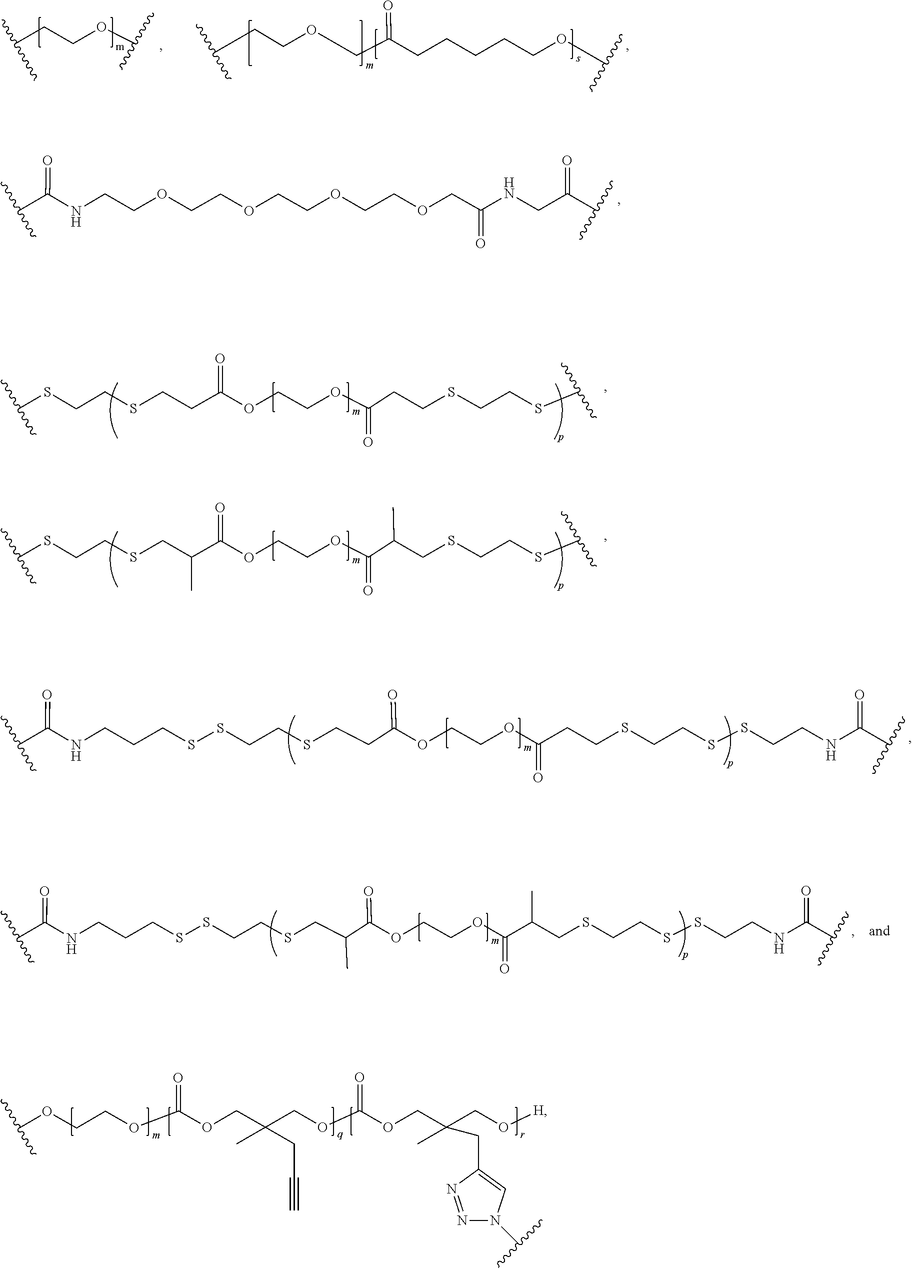

wherein:
m is an integer selected from 1 to 5;
p is an integer selected from 1 to 3;
q is an integer selected from 1 to 5;
r is an integer selected from 1 to 5; and
s is an integer selected from 1 to 5
wherein L is further covalently linked to C2; and

C2 is selected from PTK787, aflibercept, Brolucizumab, Triamcinolone, Dexamethasone, OPT-302, and DE-122.

In some embodiments, the compound of formula (I) is a bindarit-resveratrol conjugate. In some embodiments, the bindarit-resveratrol conjugate is of formula (IV). In some embodiments, the bindarit-resveratrol conjugate is of formula (V).

In embodiments, the compound of the present disclosure is a compound of formula (IV), or a pharmaceutically acceptable salt thereof:

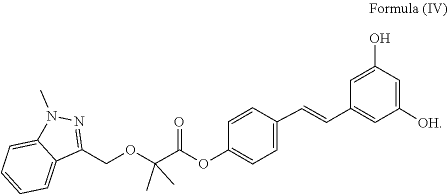 [SMILES: Cn1nc(COC(C)(C)C(=O)Oc2ccc(/C=C/c3cc(O)cc(O)c3)cc2)c2ccccc21]

In embodiments, the compound of the present disclosure is a compound of formula (V), or a pharmaceutically acceptable salt thereof:

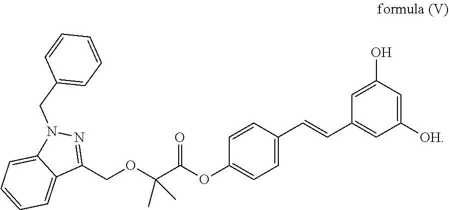 [SMILES: CC(C)(OCc1nn(Cc2ccccc2)c2ccccc12)C(=O)Oc1ccc(/C=C/c2cc(O)cc(O)c2)cc1]

In some embodiments, the compound of formula (I) is a bindarit-taurine conjugate. In some embodiments, the bindarit-taurine conjugate is of formula (VI).

In embodiments, the compound of the present disclosure is a compound of formula (VI), or a pharmaceutically acceptable salt thereof:

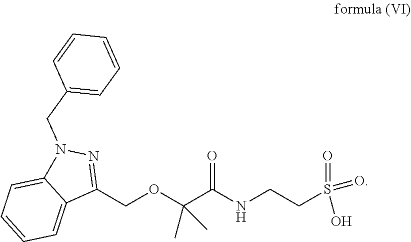 [SMILES: CC(C)(OCc1nn(Cc2ccccc2)c2ccccc12)C(=O)NCCS(=O)(=O)O]

In some embodiments, the compound of formula (I) is a bindarit-carnosine conjugate. In some embodiments, the bindarit-carnosine conjugate is of formula (VII). In some embodiments, the bindarit-carnosine conjugate is of formula (VIIa). In some embodiments, the bindarit-carnosine conjugate is of formula (VIIb).

In embodiments, the compound of the present disclosure is a compound of formula (VII), or a pharmaceutically acceptable salt thereof:

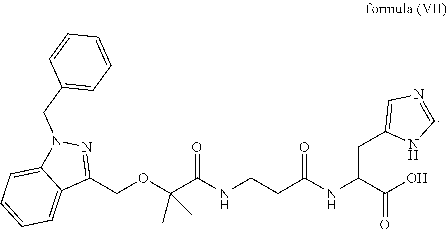 [SMILES: CC(C)(OCc1nn(Cc2ccccc2)c2ccccc12)C(=O)NCCC(=O)NC(Cc1cnc[nH]1)C(=O)O]

In embodiments, the compound of the present disclosure is a compound of formula (VIla), or a pharmaceutically acceptable salt thereof:

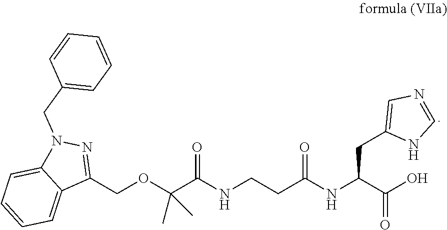 [SMILES: CC(C)(OCc1nn(Cc2ccccc2)c2ccccc12)C(=O)NCCC(=O)N[C@@H](Cc1cnc[nH]1)C(=O)O]

In embodiments, the compound of the present disclosure is a compound of formula (VIlb), or a pharmaceutically acceptable salt thereof:

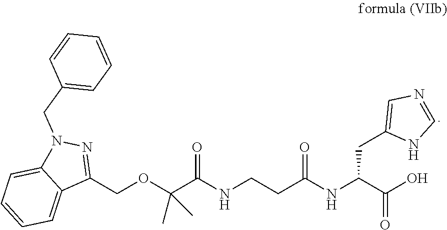 [SMILES: CC(C)(OCc1nn(Cc2ccccc2)c2ccccc12)C(=O)NCCC(=O)N[C@H](Cc1cnc[nH]1)C(=O)O]

In embodiments, the compound comprises a conjugate comprising bindarit and a nutraceutical.

In embodiments, the compound comprises a conjugate comprising bindarit and a peptide.

In embodiments, the compound comprises a conjugate comprising bindarit and a protein.

### Methods of the Disclosure

Accordingly, in one aspect, the disclosure provides a method for treating or preventing an ocular disease or disorder, comprising administering to a subject in need thereof a pharmaceutical composition comprising an effective amount of one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005. In some aspects, the disclosure relates to a method for treating or preventing an ocular disease or disorder, comprising administering to a subject in need thereof a pharmaceutical composition comprising an effective amount of one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, wherein the subject has abnormal (e.g. increased or decreased) expression or activity of one or more of CD64, IDO, SOCS1, CXCL10 Marco, Nos2, II12b, Ptgs2 (Cox2), MCP-1 (CCL2), MCP-2 (CCL8), MCP-3 (CCL7), 1123a (1123p19), Ido1, Adipoq, Ccl20, IL17 (subtype a)Ccl5, CD163, Cx3Cr1, Faslg, Gfap, Csf2, Icam1, Ifng, 1110, 1112b, II13, 1117, 1118, Il1b, 1122, 114, 116, Klf4, Mrc1, Myd88, Nlrp3, Ppary, Tgfb1, Tlr4, Tnf, Vcam1, Ccl2, Ccl5, Ccl7, Ccr2, Socs1, Socs3, Stat1, Stat3, and Stat6.

An ocular disease or disorder refers to one of various ophthalmic diseases and includes diseases of the eye and the ocular adnexa. In some embodiments, the ocular disease or disorder is, for example, a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; Wegener granulomatosis, optic neuritis, optionally wherein the optic neuritis is primary or secondary to systemic (i.e. non-ocular) disease, wet AMD, dry AMD, RPD, white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), diffuse subretinal fibrosis (DSF)), LORDs, and central serous retinopathy (CSR). An ocular disease may also include diseases of the optic nerve, a structure that carries visual information to and from the brain. By way of non-limiting example, these may include optic neuropathy that may be primary (i.e., result from disease of the nerve itself, or may be secondary to another disorder such as multiple sclerosis), optic neuritis, optic nerve inflammation, optic nerve damage, optic nerve trauma.

In some embodiments, the dry AMD is early stage AMD or atrophic AMD. In embodiments, the AMD is or comprises intermediate AMD. In embodiments, intermediate AMD comprises imminent risk of progression to late disease. In some embodiments, the method of the present disclosure prevents and/or reduces the rate of progression to late atrophic disease. In some embodiments, the method of the present disclosure prevents and/or reduces the progression to late AMD. In some embodiments, the method of the present disclosure prevents and/or reduces the rate of expansion of geographic atrophy. In some embodiments, the method of the present disclosure prevents the onset of atrophy. In some embodiments, the method of the present disclosure prevents and/or reduces the progression to late atrophic disease. In some embodiments, the method of the present disclosure prevents and/or reduces the progression of angiogenesis and/or atrophy.

In embodiments, atrophy comprises eyes with incomplete outer retinal atrophy (iORA), eyes with complete outer retinal atrophy (cORA), eyes with incomplete retinal pigment epithelium (RPE) eyes with outer retinal atrophy (iRORA), and/or eyes with complete RPE and ORA (cRORA). In embodiments, atrophy comprises eyes with progressive thinning of the outer retina. In embodiments, the progressive thinning of the outer retina is captured and/or identified using in vivo imaging such as, but not limited to optical coherence tomography (OCT), en face OCT, or optical coherence tomography angiography (OCT-A).

In some embodiments, the onset of geographic atrophy in the subject is assessed using optical coherence tomography (OCT).

In some embodiments, the method further comprises reducing the subject's expression of one or more of CD64, IDO, SOCS1, CXCL10 Marco, Nos2, 1112b, Ptgs2 (Cox2), MCP-1 (CCL2), MCP-2 (CCL8), MCP-3 (CCL7), 1123a (H23p19), Idol, Adipoq, Ccl20, IL17 (subtype a)Ccl5, CD163, Cx3Cr1, Faslg, Gfap, Csf2, Icaml, Ifng, 1110, 1112b, 1113, 1117, 1118, 1M b, 1122, 114, 116, Klf4, Mrd, Myd88, Nlrp3, Pparv, Tgfb1, Tlr4, Tnf, Vcaml, Ccl2, Ccl5, Ccl7, Ccr2, Socsl, Socs3, Statl, Stat3, and Stat6.

In some embodiments, the subject has a modulated (e.g. decreased or increased) expression or activity of one or more of MRC1, TGM2, CD23, CCL22 Relma (Fizz1, Retnla), Socs2, Irf4, Chia (Amcase), Chi311 (Gp39, Ykl40), Chi312 (Ykl39), Chi313 (Ym1), Cxcl13, Ccl12, Ccl24, and Klf4.

In some embodiments, the gene responses mitigated by bindarit (and therefore useful markers of the disclosure) are one or more of: Adipoq, ApoE, Timp3, Gfap, Nlrp3, Ccl11 (Eotaxin1), Cx3Cr1, Socs3, Stat1, Ccl20, ∥17a, RPE65, Cfb, Clqb, Serping1, C3, C4b, IL-6, Myd88, and C6.

In some embodiments, the ocular disease or disorder is one or more of dry AMD, RPD, white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), diffuse subretinal fibrosis (DSF)), LORDs (i.e. Q1qTNF5 deficiency), and central serous retinopathy (CSR). In some embodiments, the ocular disease or disorder is Lecithin Retinol Acyltransferase (LRAT) deficiency, which is optionally associated with: Irat-related leber congenital amaurosis, and retinal dystrophy, early-onset, severe. In some embodiments, the ocular disease or disorder is fundus albipunctatus, which may be associated with one or more of the following genetic locations: 3q22.1 (Retinitis punctata albescens, RHO); 6p21.1 (Retinitis punctata albescens, PRPH2); 12q13.2 (Fundus albipunctatus, RDH5); 15q26.1 (Retinitis punctata albescens, RLBP1); and 15q26.1 (Fundus albipunctatus, RLBP1). In some embodiments the ocular disease or disorder is one or more of Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; Wegener granulomatosis, and optic neuritis, optionally wherein the optic neuritis is primary or secondary to systemic (i.e. non-ocular) disease.

In some embodiments, the ocular disease or disorder is one or more of a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; idiopathic uveitis, Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; Wegener granulomatosis, and optic neuritis, optionally wherein the optic neuritis is primary or secondary to systemic (i.e. non-ocular) disease.

In some embodiments, the subject is not undergoing treatment with and/or is unresponsive and/or is substantially unresponsive and/or is partially responsive one or more of an anti-factor D antibody (e.g. lampalizumab (Genentech)), an anti-β-amyloid (anti-AB) antibody (e.g. GSK933776 (GSK), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), Zimura (Iveric), an anti-C3 complement antibody (e.g. APL-2 (Apellis) or related compounds thereof, NGM621 (NGM Biopharmaceutical)), doxycycline (ORACEA), emixustat hydrochloride, rapalogues (e.g. sirolimus (RAPAMUNE)), metformin or analogues thereof, and glatiramer acetate (COPAXONE). In some embodiments, the subject has evidence of AMD as confirmed by the presence of at least 1 druse greater than about 125 μm in diameter and/or pigment. In some embodiments, the subject has early stage dry macular degeneration as evidenced bysmall drusen and/or medium-sized drusen and/or large drusen and/or reticular pseudodrusen, and/or late AMD and/or dry AMD as evidence by nascent, incomplete, emerging or evolving geographic atrophy and/or complete geographic atrophy. In some embodiments, the subject has early, intermediate, or late stage dry macular degeneration as evidenced by a large number of medium-sized drusen, pigment, and/or atrophy. In some embodiments, the subject has early AMD with drusen, intermediate AMD with drusen and/or pigment, or late dry macular degeneration as evidenced first by nascent, incomplete, emerging, evolving geographic atrophy, and complete geographic atrophy that continues to expand over time In some embodiments, the subject has reticular pseudodrusen, or early or late stage dry AMD as evidenced by subretinal drusenoid deposits. In some embodiments, the subject has a variant of AMD. In embodiments, the subject has atrophic AMD. Non-limiting examples of a variant of AMD include polypoidal choroidal vasculopathy (PCV) and retinal angiomatous proliferation (RAP). In some embodiments, the subject may or may not have evidence of prior or concurrent active choroidal neovascularization (CNV). In some embodiments, the subject has one or more disease features suggesting the subject is at risk for CNV such as sub- or intra-retinal fluid, sub-RPE fluid, non-exudative CNV, and a double-layer sign. See, e.g., Shi et al. Ophthalmology Retina 3, P211-P219 (2019), which is incorporated by reference herein. In some embodiments, the subject has one or more well-demarcated GA lesions of a total area of about 2 to about 20 mm² in one or more eye and/or 0.5 to 10 disc areas (e.g. 0.5 to 7, or about 0.5, or about 1, or about 2, or about 3, or about 4, or about 5, or about 6, or about 7, or about 8, or about 9, or about 10 disc areas), about 0.2 to 2.0 to about 20 mm² using en face imaging in one or more eye, and/or a diameter of below about 200 μm, between about 200 and about 250 μm, or greater than about 250 μm, optionally measured using cross-sectional OCT. In some embodiments, the subject has a best-corrected visual acuity score of greater than about 35 letters or a Snellen VA equivalent of about 20/200 or better. In some embodiments, the subject has a best-corrected visual acuity score of greater than about 35 letters or a Snellen VA equivalent of about 20/400 or better. In In some embodiments, the subject has early stage dry macular degeneration as evidenced by large drusen and pigmentary change.

In various aspects, the present methods are applicable to select subject populations. For example, the present methods, including embodiments in which the agent is of one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, and the ocular disease or disorder is dry AMD or RPD, the subject is not undergoing treatment with and/or is unresponsive and/or is substantially unresponsive and/or is partially responsive to one or more of an anti-factor D antibody (e.g. lampalizumab (Genentech)), an anti-B-amyloid (anti-AB) antibody (e.g. GSK933776 (GSK), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), Zimura (Iveric), an anti-C3 complement antibody (e.g. APL-2 (Apellis) or related compounds thereof, NGM621 (NGM Biopharmaceutical)), doxycycline (ORACEA), emixustat hydrochloride, rapalogues (e.g. sirolimus (RAPAMUNE), metformin or analogues thereof, and glatiramer acetate (COPAXONE) . . . . In various embodiments, including embodiments in which the agent is bindarit and the ocular disease or disorder is dry AMD or RPD, the subject is classified by having one or more of the following: at least 1 druse greater than about 125 μm in diameter and/or pigment, no evidence of prior or active choroidal neovascularization (CNV), one or more well-demarcated GA lesions of a total area of about 2 to about 20 mm² in one or more eye, about 0.2 to 2.0 to about 20 mm² using en face imaging in one or more eye, and/or a diameter of below about 200 μm, between about 200 and about 250 μm, or greater than about 250 μm, optionally measured using cross-sectional OCT, a best-corrected visual acuity score of greater than about 35 letters or a Snellen VA equivalent of about 20/200 or better, and a macular photocoagulation study disc area of less than about 7.0, and less than 1 disc up to more than 10 disc areas (e.g. about 1, or about 2, or about 3, or about 4, or about 5, or about 6, or about 7, or about 8, or about 8, or about 9, or about 10 disc areas). In various embodiments, the subject is classified as having one or more of: at least 1 large druse, or RPD, and hypofluorescent (dark or black) regions identified by DNIRA of a total area of about 2 mm² to about 20 mm² in one or more eye, or less than 1 disc areas or up to more than 10 disc areas (e.g. about 1, or about 2, or about 3, or about 4, or about 5, or about 6, or about 7, or about 8, or about 8, or about 9, or about 10 disc areas) of hypofluorescent DNIRA signal, in one or more eye. In some embodiments, the subject has a GA lesion of less than about 200 μm diameter up to more than about 500 μm. In one aspect, the disclosure provides a method for treating or preventing an ocular disease or disorder, comprising administering to a subject in need thereof an effective amount of one or more of a compound of formula (I), formula (Ia), formula (Ib), formula (Ic), formula (Id), formula (Ie), formula (Ia1), formula (Ia2), formula (la3), formula (If), formula (Ig), formula (Ih), formula (Ii), formula (Ij), formula (Ik), formula (Im), formula (In), formula (lia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, or a pharmaceutically acceptable salt thereof, as described herein. In some embodiments, the ocular disease or disorder is one or more of dry AMD, RPD, white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), diffuse subretinal fibrosis (DSF)), LORDs, and central serous retinopathy (CSR). In some embodiments, the ocular disease or disorder is one or more of a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; Wegener granulomatosis, and optic neuritis, optionally wherein the optic neuritis is primary or secondary to systemic (i.e. non-ocular) disease. In some embodiments, the disclosure provides a method for treating or preventing an ocular disease or disorder, comprising administering to a subject in need thereof an effective amount of one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (lic), formula (i2a), formula (i2b), formula (IIIa), formula (IIIb), formula (IV), formula (V), formula (VI), formula (VII), formula (VIIa), formula (VIIb), and formulas 1001 to 1005 or a pharmaceutically acceptable salt thereof, wherein the ocular disease or disorder is selected from a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; Wegener granulomatosis, and optic neuritis, optionally wherein the optic neuritis is primary or secondary to systemic (i.e. non-ocular) disease.

In a further aspect, the disclosure provides a method for treating or preventing an ocular disease or disorder, comprising administering to a subject in need thereof an effective amount of a compound of one or more of a compound of formula (I), formula (Ia), formula (Ib), formula (Ic), formula (Id), formula (Ie), formula (Ia1), formula (la2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, or a pharmaceutically acceptable salt thereof, wherein the ocular disease or disorder is selected from a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; Wegener granulomatosis, and optic neuritis, optionally wherein the optic neuritis is primary or secondary to systemic (i.e. non-ocular) disease.

In a further aspect, the disclosure provides a method for treating or preventing an ocular disease or disorder, comprising administering to a subject in need thereof an effective amount of a compound of one or more of a compound of formula (I), formula (Ia), formula (Ib), formula (Ic), formula (Id), formula (Ie), formula (Ia1), formula (la2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, or a pharmaceutically acceptable salt thereof, wherein the ocular disease or disorder is selected from a cataract, glaucoma, or optic neuropathy.

In some embodiments, the patient has type 1 or type 2 diabetes. In various embodiments, the patient has a blood glucose level that exceeds normal levels (about 70-130 milligrams per deciliter or mg/dl before meals, and less than 180 mg/dL one to two hours after a meal).

In some embodiments, the diabetic eye disease is diabetic retinopathy in one or more of the following stages: Mild Nonproliferative Retinopathy (in which microaneurysms may occur); Moderate Nonproliferative Retinopathy (in which some blood vessels that nourish the retina may be blocked); and Severe Nonproliferative Retinopathy (in which more blood vessels may be blocked, depriving several areas of the retina with their blood supply); and Proliferative Retinopathy (in which signals sent by the retina for nourishment trigger the growth of new blood vessels.

In other embodiments, the present disclosure pertains to treatment or prevention of a cataract glaucoma, or optic neuropathy comprising administering to a patient in need thereof an effective amount of one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, or a pharmaceutically acceptable salt thereof.

In some embodiments, one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, or a pharmaceutically acceptable salt thereof is useful for treating, preventing, or reducing the rate of pathogenesis of an ocular disease or disorder.

In various embodiments, the ocular disease or disorder is AMD. AMD is the leading cause of blindness in the developed world. Early AMD is characterized by the accumulation of drusen, the hallmark of disease, while intermediate AMD includes, in various definitions, large drusen and pigmentary change. Geographic atrophy (GA) and choroidal neovascularization (CNVM) include the late blinding complications of late non-exudative (so-called “dry” or atrophic) and exudative (so-called “wet” or neovascular) disease, respectively. Anti-VEGF therapy has revolutionized treatment of wet AMD, but represents just 12-15% of all AMD cases. Though vitamin supplementation can slow the rates of progression no treatments exist for dry AMD.

In various embodiments, the ocular disease or disorder is early non-exudative or “dry” AMD. Early non-exudative or “dry” AMD can be characterized by drusen accumulation and associated features such as pigmentation change, or RPE detachments. Late, or advanced dry AMD can be characterized by patchy atrophy of the RPE and overlying photoreceptors. These patches are visualized clinically as so-called “window defects” and are known as areas of “geographic atrophy.” In embodiments, the ocular disease may also include emerging or evolving stages of geographic atrophy that are considered nascent, or incomplete in its early stages, and complete when it fully includes both the retinal pigment epithelium and outer retina of a minimum size, such as about 100 μm, about 200 μm, about 250, or about 300 μm.

In various embodiments, the ocular disease or disorder is wet AMD. Wet AMD is characterized by the growth of new blood vessels beneath the retina or RPE which can bleed and leak fluid, resulting in a rapid and often severe loss of central vision in the majority cases. This loss of central vision adversely affects one's everyday life by impairing the ability to read, drive and recognize faces. In some cases, the macular degeneration progresses from the dry form to the wet form.

In one embodiment, the methods pertain to treatment of exudative or wet AMD and dry AMD simultaneously in the same patient.

In one embodiment, the ocular disease or disorder is associated with choroidal neovascularization (CNVM).

In some embodiments, the present disclosure pertains to prevention or treatment of dry AMD, which may be identifiable by the presence of areas of hyper-fluorescent FAF in an eye of the subject and/or the presence of one or more areas of abnormally fluorescent FAF in an eye of the subject and/or by changes in one or more of blue spectrum fundus imaging, white-light fundus imaging, red-free fundus imaging, and OCT in an eye of the subject and/or by an increase (including a transient increase) in permeability across the subject's epithelial barrier between a choroid and a retina relative to an undiseased state and/or by a deformation of the outer retina, and/or deformation of the mid-retinal vasculature and outward subsidence of the outer retina, across the subject's epithelial barrier between a choroid and a retina relative to an undiseased state and/or by the presence of phagocytic immune cells across the subject's RPE relative to an undiseased state.

In some embodiments, the dry AMD is early stage AMD, or is late stage atrophic dry AMD. In embodiments, the AMD is atrophic AMD.

In other embodiments, the subject is a human. In still other embodiments, the administering is effected orally or intra-vascularly, or intraocularly, or periocularly, or to the ocular surface.

In another aspect, the disclosure provides a method of treating RPD disease, comprising administering to a subject in need thereof a pharmaceutical composition comprising an effective amount of one or more of a compound of formula (I), formula (Ia), formula (Ib), formula (Ic), formula (Id), formula (Ie), formula (Ia1), formula (la2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, or a pharmaceutically acceptable salt thereof. RPD disease can be also known as, but not limited to, the following: “reticular drusenoid disease,” “pseudoreticular drusen,” and “drusenoid macular disease,” or “disease characterized by the presence of subretinal drusenoid deposits.” Not wishing to be bound by theory, RPD may be a subtype of, or may be related to AMD. Both AMD and RPD can lead to the blinding complications of choroidal neovascularization or geographic atrophy.

In some embodiments, the RPD disease is that in which pseudodrusen material is reduced or eradicated or the accumulation and/or expansion of which is slowed upon treatment. In embodiments, the method of the present disclosure comprises reducing the amount of pseudodrusen in the subject. In some embodiments, the disclosure provides a method for treating RPD disease in which the pseudodrusen are reduced or eradicated upon treatment in the foveal area and/or perifoveal area and/or juxtafoveal area of a subject's eye. Any method useful for assessing and/or measuring a reduction in the amount of pseudodrusen is contemplated by the present disclosure, as would be understood by one of ordinary skill in the art. Non-limiting examples of methods useful to assess and/or measure a reduction in the amount of pseudodrusen in a subject includes optical coherence tomography (OCT).

In embodiments, the method of the present disclosure comprises reducing expansion of pseudodrusen in the subject. In embodiments, the method of the present disclosure comprises reducing expansion of pseudodrusen in any one of the foveal area, perifoveal area, and juxtafoveal area of the subject's eye.

In some embodiments, the disclosure further provides methods for treatment or prevention of RPD disease in which the rates of progression from early to late disease are reduced. Late disease can include, for example, choroidal neovascularization or geographic atrophy. In embodiments, the method of the present disclosure prevents and/or reduces the rate of progression to late disease. In embodiments, the method of the present disclosure prevents and/or reduces the progression to late AMD. In embodiments, the method of the present disclosure prevents and/or reduces the rate of expansion of geographic atrophy. In embodiments, the method of the present disclosure prevents the onset of atrophy. In embodiments, the method of the present disclosure prevents and/or reduces the progression to late atrophic disease. In embodiments, the method of the present disclosure prevents and/or reduces the progression of angiogenesis and/or atrophy.

In some embodiments, the disclosure further provides methods for treatment or prevention of RPD disease in which the rates of expansion of geographic atrophy are reduced.

In still other embodiments, the RPD disease is identifiable by the presence of one or more areas of distinct patterns of retinal imaging in the eye of a subject, wherein the retinal imaging is one or more of white light, red-free light, blue light, FAF, near infra-red (NIR), infra-red (IR), angiography, and DNIRA and/or the presence of one or more areas of abnormally-fluorescent FAF in the eye of a subject and/or an increase (including a transient increase) in permeability across the subject's epithelial barrier between a choroid and a retina relative to an undiseased state and/or a presence of phagocytic immune cells across the subject's RPE relative to an undiseased state

In embodiment, the onset of geographic atrophy in the subject is assessed using optical coherence tomography (OCT).

In some embodiments, the methods described herein comprise reducing the amount of pseudodrusen in the subject and/or reducing the amount of pseudodrusen in any one of the foveal area, perifoveal area, juxtafoveal area, and extrafoveal area of the subject's eye. In other embodiments, the methods described herein comprise reducing the rates of progression to late disease, wherein the late disease is any one of choroidal neovascularization or geographic atrophy. In some embodiments, the methods described herein comprise reducing the rates of expansion of geographic atrophy. In some embodiments, the methods described herein comprise reducing translocation of, or activity of, nuclear receptors, to or within the nucleus in the eye cells of the subject, wherein reducing translocation or activity results in the alteration of transcriptional activity. In some embodiments, the methods described herein comprise normalizing cellular stress in cells under hypoxic, oxidative, superoxide, nitrosyl or glycemic stress. In some embodiments, the methods described herein comprise reducing NFkb translocation to the nucleus in eye cells of the subject.

In some embodiments, the method further comprises reducing, increasing or otherwise modulating the activity of lipid transport molecules, including but not limited to Fatty Acid Binding Proteins (FABPs), Nuclear Receptor/Transcription Factors (NR/TFs), transmembrane channels, and/or lipid enzymes. In a non-limiting example, collectively lipid transport molecules share a beta-barrel motif in which lipids, including but not limited to fatty acids, can be transported across aqueous environments such as the cytoplasm, and in the case of transmembrane channels, provide selectivity and barriers to random diffusion of lipidic molecules therein.

In embodiments, bindarit and analogues thereof are Fatty Acid Mimetic (FAM) molecules. In some embodiments, bindarit and analogues thereof dock within the beta-barrel domain. In some embodiments, bindarit and analogues thereof interact with about 3, about 4, about 5, about 6, about 7, or about 8 sites within the pocket of the beta barrel domain. In some embodiments, bindarit and analogues thereof interact and/or bind with specificity to about 3, about 4, about 5, about 6, about 7, or about 8 sites within the pocket of the beta barrel domain compared to binding at other sites of the protein. In some embodiments, the about 3, about 4, about 5, about 6, about 7, or about 8 sites within the pocket of the beta barrel domain provide specificity to lipid or lipid-mimetic transport of Fatty Acid Mimetic (FAM) molecules, including but not limited to bindarit and analogues thereof.

In some embodiments, C1 and its derivatives bind to and/or within one or more Fatty Acid Binding Protein (FAPB), including but not limited to, FABP-1, FABP-2, FABP-3, FABP-4, FABP-5, FABP-6, FABP-7, FABP-8, FABP-9, FABP-10, FABP-11, and FABP-12. In some embodiments, C1 and its derivatives bind to and/or within FABP-1. In some embodiments, C1 and its derivatives bind to and/or within FABP-2. In some embodiments, C1 and its derivatives bind to and/or within FABP-3. In some embodiments, C1 and its derivatives bind to and/or within FABP-4. In some embodiments, C1 and its derivatives bind to and/or within FABP-5. In some embodiments, C1 and its derivatives bind to and/or within FABP-6. In some embodiments, C1 and its derivatives bind to and/or within FABP-7. In some embodiments, C1 and its derivatives bind to and/or within FABP-8. In some embodiments, C1 and its derivatives bind to and/or within FABP-9. In some embodiments, C1 and its derivatives bind to and/or within FABP-10. In some embodiments, C1 and its derivatives bind to and/or within FABP-11. In some embodiments, C1 and its derivatives bind to and/or within FABP-12. In some embodiments, C1 and its derivatives bind to and/or within FABP-4 and the amino acid residues that the C1, C2 and/or L binds and/or interacts with are one or more selected from PHE-17, Ser-54, Thr-61, GLU-73, ARG-79, ASP-77. In some embodiments, an amino acid that binds and/or interacts with one or more FABP is within about 3 to about 4 angstroms, about 3 angstroms, about 3.1 angstroms, about 3.2 angstroms, about 3.3 angstroms, about 3.4 angstroms, about 3.5 angstroms, about 3.6 angstroms, about 3.7 angstroms, about 3.8 angstroms, about 3.9 angstroms, or about 4 angstroms from C1, C2 and/or L.

In some embodiments, C1 and its derivatives bind to Nuclear Receptor/Transcription Factors (NR/TFs). Non-limiting examples of NR/TFs include the PPAR family, estrogen receptors, androgen receptors, vitamin D receptors, vitamin A receptors, corticosteroid receptors, mineralacorticosteroid receptors, retinoic acid receptors, LXR family receptors, and RXR. In some embodiments, C1 and its derivatives bind to the PPAR family. In some embodiments, C1 and its derivatives bind to estrogen receptors. In some embodiments, C1 and its derivatives bind to androgen receptors. In some embodiments, C1 and its derivatives bind to vitamin D receptors. In some embodiments, C1 and its derivatives bind to vitamin A receptors. In some embodiments, C1 and its derivatives bind to corticosteroid receptors. In some embodiments, C1 and its derivatives bind to mineralacorticosteroid receptors. In some embodiments, C1 and its derivatives bind to retinoic acid receptors. In some embodiments, C1 and its derivatives bind to LXR family receptors. In some embodiments, C1 and its derivatives bind to RXR.

In some embodiments, C1 and its derivatives are directly or indirectly conjugated to non-steroidal anti-inflammatory drugs (NSAIDs). In some embodiments, C1 and its derivatives are directly conjugated to non-NSAIDs. In some embodiments, C1 and its derivatives are indirectly conjugated to NSAIDs. In some embodiments, NSAIDs include indomethacin (Indocin), ibuprofen, diclofenac (Voltaren), acemetacin, diclofenac, naproxen, ketorolac, carprofen, etodolac, fentiazac, flurbiprofen, ketoprofen, oxaprozin, pranoprofen, sulindac, suxibuzone, celecoxib (Celebrex®), fenoprofin (Nalfon), ketorolac tromethamine, tolmetin, diflunisal, meclofenamate sodium, mefenamic acid, etoricoxib, meloxicam, nabumetone, piroxicam, or derivatives thereof. In some embodiments, the NSAID is indomethacin (Indocin). In some embodiments, the NSAID is ibuprofen. In some embodiments, the NSAID is diclofenac (Voltaren). In some embodiments, the NSAID is acemetacin. In some embodiments, the NSAID is diclofenac diclofenac. In some embodiments, the NSAID is naproxen. In some embodiments, the NSAID is ketorolac. In some embodiments, the NSAID is carprofen. In some embodiments, the NSAID is etodolac. In some embodiments, the NSAID is fentiazac. In some embodiments, the NSAID is flurbiprofen. In some embodiments, the NSAID is ketoprofen. In some embodiments, the NSAID is oxaprozin. In some embodiments, the NSAID is pranoprofen. In some embodiments, the NSAID is sulindac. In some embodiments, the NSAID is suxibuzone. In some embodiments, the NSAID is celecoxib (Celebrex®). In some embodiments, the NSAID is fenoprofin (Nalfon). In some embodiments, the NSAID is ketorolac tromethamine. In some embodiments, the NSAID is tolmetin. In some embodiments, the NSAID is diflunisal. In some embodiments, the NSAID is meclofenamate sodium. In some embodiments, the NSAID is mefenamic acid. In some embodiments, the NSAID is etoricoxib. In some embodiments, the NSAID is meloxicam. In some embodiments, the NSAID is nabumetone. In some embodiments, the NSAID is piroxicam. In some embodiments C2 includes acetylsalicylic acidacid (aspirin). In some embodiments, the NSAID acts as a FAM. In some embodiments, the NSAID sits within the beta-barrel domain. In some embodiments, the NSAID thereof interacts with about 3, about 4, about 5, about 6, about 7, or about 8 sites within the pocket of the beta barrel domain. In some embodiments, the linker is a cleavable linker. In some embodiments, the linker between C1 and an NSAID is cleaved by an enzyme. In some embodiments, the linker between C1 and an NSAID is cleaved by a hydrolase enzyme. In some embodiments, the linker between C1 and an NSAID is cleaved by an esterase. In some embodiments, the linker between C1 and an NSAID is cleaved by a lipase. In some embodiments, the linker between C1 and an NSAID is cleaved by a phosphatase. In some embodiments, the linker between C1 and an NSAID is cleaved by a glycosidase. In some embodiments, the linker between C1 and an NSAID is cleaved by a peptidase. In some embodiments, the linker between C1 and an NSAID is cleaved by a nucleosidase. In some embodiments, an esterase is activated to cleave compounds exclusively and/or predominantly upon disease activity. By way of non-limiting example, such activation may occur during catalytic cleavage of the complement cascade, or the clotting cascade. In some embodiments, such cleavage may occur due to the activity of lipases. In some embodiments, C1 and its derivatives, including those with and without linkage to other compounds, (e.g. C2, C3, C4, etc.) may be preferentially targeted by lipases. In some embodiments, the lipases comprise beta-barrel domains for lipid handling.

In some embodiments, C1 and its derivatives are ester-linked compounds. In some embodiments, ester-linked compounds are lipid-binding molecules. In some embodiments, C1 and its derivatives and/or ester-linked compounds and/or lipid-binding molecules of the disclosure comprise a structure including, but not limited to, C1-(ester)ₙ-C1, C1-(ester)ₙ-C2, C1-(ester)ₙ-C3, and/or C1-(ester)ₙ-C2-(ester)ₙ-C3, wherein n is an integer from 0 to 20. In some embodiments, C1, C2, L, C1-La, C1-C₂-L1, and/or C1-C₂-L2 comprise modifications. In some embodiments, C1, C2, L, C1-La, C1-C₂-L1, and/or C1-C₂-L2 comprise modifications (e.g. deuteration) at a position and/or atom that does not interact with the about 3, about 4, about 5, about 6, about 7, or about 8 sites within the pocket of the beta barrel domain. In some embodiments, the modifications are chemical modifications. In some embodiments, the modifications comprise and/or consist of deuteration. In some embodiments, at least 1 hydrogen is replaced with deuterium. In some embodiments, about 1, about 2, about 3, about 4, about 5, about 6, about 7, about 8, about 9, or about 10 hydrogens are replaced with deuterium. In some embodiments, any hydrogen in C1, C2, L, C1-La, C1-C2-L1, and/or C1-C2-L2 can be replaced with deuterium. In some embodiments, modifications may include linkage or modification at a position of the molecule that binds to and/or within the beta-barrel domain. In some embodiments, modification may include linkage or modification at a position of the molecule that does not bind to and/or within the beta-barrel domain. In some embodiments, modification may occur at points in the molecule relevant to binding at the alpha-helix. In some embodiments, modification may occur randomly within the channel of the beta-barrel.

In some embodiments, the disclosure provides a method for identifying a subject having an ocular disease or disorder, wherein the subject is more likely than not to respond to treatment with an agent comprising determining whether the subject's eye has, or previously had, an increase (including a transient increase) in permeability across the epithelial barrier between a choroid and a retina of the eye relative to an undiseased state; wherein the increase in permeability indicates that the subject is more likely than not to respond to treatment with the agent; and wherein the agent is one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, or a pharmaceutically acceptable salt thereof.

In some embodiments, the ocular disease or disorder is one or more of a diabetic eye disease, dry AMD and RPD disease.

In other embodiments, the diabetic eye disease is diabetic retinopathy or DME.

In another embodiment, the ocular disease or disorder is LORDs (late onset retinal degeneration) or retinal degeneration associated with c1qTNF5 deficiency or its corresponding gene mutation, or another maculopathy, including, but not limited to, Stargart disease, pattern dystrophy, as well as retinitis pigmentosa (RP) and related diseases. In one embodiment, the maculopathy is inherited.

In other embodiments, the blinding eye disease is an idiopathic disorder that may, without wishing to be bound by theory, be characterized by retinal inflammation, with or without accompanying macular degeneration, including, but not limited to, white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), and diffuse subretinal fibrosis (DSF)).

In other embodiments, the ocular disease or disorder is central serous retinopathy (CSR). CSR is a fluid detachment of macula layers from their supporting tissue. CSR is often characterizable by the leak and accumulation of fluid into the subretinal or sub-RPE space. Without wishing to be bound by theory, the leak and accumulation of fluid may occur because of small breaks in the RPE.

In some embodiments, the ocular disease or disorder may be of a certain stage or progression. In some embodiments, the ocular disease or disorder may be incipient, emerging, quiescent, advancing or active.

In addition to treating pre-existing ocular diseases or disorders, the present disclosure comprises prophylactic methods in order to prevent or slow the onset of these disorders. In prophylactic applications, an agent can be administered to a subject susceptible to or otherwise at risk of a particular ocular disease or disorder. Such susceptibility may be determined by, for example, familial predisposition, genetic testing, risk factor analysis, blood or other cytokine or biomarker levels, and ocular examination, which can include multi-modal analysis such as FAF, blue light, white light, red-free, near infra-red, infrared, DNIRA, etc. Such susceptibility may also be determined by, for example, detection by OCT, with cross-sectional, three-dimensional and en face viewing.

In some embodiments, the ocular disease or disorder is one or more ailments listed in the International Statistical Classification of Diseases and Related Health Problems, or ICD-10 in the following sections (which are incorporated herein by reference): H30 chorioretinal inflammation, H31 other disorders of choroid, H32 chorioretinal disorders in diseases classified elsewhere, H33 retinal detachments and breaks, H34 retinal vascular occlusion, H35 other retinal disorders, and H36 retinal disorders in diseases classified elsewhere. In some embodiments the ocular disease or disorder is one of the optic nerve.

In some embodiments, the present methods for treatment or prevention of an ocular disease or disorder (e.g. a diabetic eye disease (for instance, diabetic retinopathy and DME), Vogt-Kayanagi-disorder (e.g. a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; Wegener granulomatosis, and optic neuritis, optionally wherein the optic neuritis is primary or secondary to systemic (i.e. non-ocular) disease) further comprise performing laser surgery (e.g. scatter laser treatment, focal laser treatment, photocoagulation) and/or vitrectomy (e.g. in which blood is removed from the center of a patient's eye) and/or administering a steroid and/or administering an anti-VEGF agent such as Ranibizumb (LUCENTIS), Bevacizumab (AVASTIN) (Aflibercept (EYLEA), KSI-301, brolucizumab (Beovu®), pegaptanib sodium (Macugen®), faricimab-svoa (VABYSMO®), ranibizumab (SUSVIMO™), MARINA, and ANCHOR, or biosimilar such as FYB201, Xlucane, LUBT010, Razumab, Ranizurel/R-TPR-024, SB11 Byooviz, SJP-0133/GBS-007, CKD-701, PF582, BCD100, MYL-1701P, ABP-938, FYB203, SB-15, SOK583A19, CT-P42, ALT-L9, OT-702, Zirabev, ABP215 (Mvasi), Cizumab, Bevacirel, BCD-021, mAbxience, Krabeva, Zybev, Abevmy, Bevatas, Cyltezo, Imraldi (EU)/Hadlima (Korea) (SB5), Amjevita (US)/Amgevita (EU)/Solymbic (EU) (ABP 501), GP2017, Exemptia (ZRC3197, Adfrar, and Mabura.

In other embodiments, the subject is a human. In still other embodiments, the administering is effected orally or intra-vascularly, or intraocularly, or periocularly, or to the ocular surface.

In some embodiments, the methods described herein further comprise administering one or more additional therapeutic agents. In some embodiments, one or more C2 groups can be administered as a part of the compounds described herein (e.g. C2 of a compound of formula (I) and/or can be administered as an additional therapeutic agent. The additional therapeutic agent can be, in various embodiments, an anti-factor D antibody (e.g. lampalizumab (Genentech)), an anti-β-amyloid (anti-AB) antibody (e.g. GSK933776 (GSK)), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), doxycycline (ORACEA), emixustat hydrochloride, sirolimus (RAPAMUNE), glatiramer acetate (COPAXONE), and an NSAID (e.g. ibuprofen, indomethacin). The additional therapeutic agent may also be, in various embodiments, an anti-VEGF agent, an ACE inhibitor, a PPAR-gamma agonist or partial agonist, a renin inhibitor, a steroid, an agent that modulates nuclear receptor activity or gene transcription, a rapalogue, metformin or analogues thereof, an agent that modulates autophagy, a semapimod, a MIF inhibitor, a CCR2 inhibitor, CKR-2B, a 2-thioimidazole, CAS 445479-97-0, CCX140, clodronate, a clodonate-liposome preparation or gadolinium chloride. The additional therapeutic agent may also be, in various embodiments, an anti-vascular endothelial growth factor (VEGF) agent, an angiotensin-converting enzyme (ACE) inhibitor, a peroxisome proliferator-activated receptor (PPAR)-gamma agonist, a renin inhibitor, a corticosteroid, and an agent that modulates autophagy. In some embodiments, the additional therapeutic agent is a modulator of the complement cascade (e.g. a modulator of C3, C5, complement factor D, or complement factor B). In various embodiments, the additional therapeutic is one or more of an anti-vascular endothelial growth factor (VEGF) agent, a modulator of the complement cascade, an angiotensin-converting enzyme (ACE) inhibitor, a peroxisome proliferator-activated receptor (PPAR)-gamma agonist, a renin inhibitor, a corticosteroid, and an agent that modulates autophagy. In various embodiments, the additional therapeutic agent is one or more of an anti-vascular endothelial growth factor (VEGF).

In some embodiments, the additional therapeutic agent is a nucleoside reverse transcriptase inhibitor (NRTIs), by way of non-limiting example zidovudine, didanosine, zalcitabine, stavudine, lamivudine, abacavir, emtricitabine, and entecavir. In some embodiments, the additional therapeutic agent is acyclovir.

In some embodiments, the additional therapeutic agent is methotrexate or a pharmaceutically acceptable salt thereof. Methotrexate has the structure:

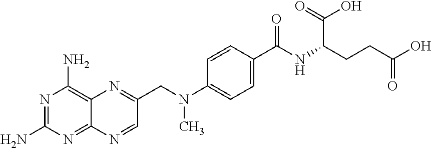 [SMILES: CN(Cc1cnc2nc(N)nc(N)c2n1)c1ccc(C(=O)N[C@@H](CCC(=O)O)C(=O)O)cc1]

Methotrexate is also known by its IUPAC name, (2S)-2-[(4-{[(2,4-diaminopteridin-6-yl)methyl](methyl)amino}benzoyl)amino]pentanedioic acid; by “MTX” and by “amethopterin.” Methotrexate sodium is available by prescription in the United States. Methotrexate's synthesis has been described in U.S. Pat. No. 4,080,325, which is hereby incorporated by reference in its entirety.

In another embodiment, the additional therapeutic agent is semapimod (CNI-1493) as described in Bianchi, et al. (March 1995). Molecular Medicine (Cambridge, Mass.) 1 (3): 254-266, the contents of which are hereby incorporated by reference in their entireties.

In still another embodiment, the additional therapeutic agent is a migration inhibitory factor (MIF) inhibitor. Illustrative MIF inhibitors are described in WIPO Publication Nos. WO 2003/104203, WO 2007/070961, WO 2009/117706 and U.S. Pat. Nos. 7,732,146 and 7,632,505, and 7,294,753 7294753 the contents of which are hereby incorporated by reference in their entireties. In some embodiments, the MIF inhibitor is (S, R)-3-(4-hydroxyphenyl)-4,5-dihydro-5-isoxazole acetic acid methyl ester (ISO—1), isoxazoline, p425 (J. Biol. Chem., 287, 30653-30663), epoxyazadiradione, or vitamin E.

In still another embodiment, the additional therapeutic agent is a chemokine receptor 2 (CCR2) inhibitor as described in, for example, U.S. Patent and Patent Publication Nos.: U.S. Pat. Nos. 7,799,824, 8,067,415, US 2007/0197590, US 2006/0069123, US 2006/0058289, and US 2007/0037794, the contents of which are hereby incorporated by reference in their entireties. In some embodiments, the CCR2) inhibitor is Maraviroc, cenicriviroc, CD192, CCX872, CCX140, 2-((Isopropylaminocarbonyl)amino)-N-(2-((cis-2-((4-(methylthio)benzoyl)amino)cyclohexyl)amino)-2-oxoethyl)-5-(trifluoromethyl)-benzamide, vicriviroc, SCH₃₅₁₁₂₅, TAK779, Teijin, RS—504393, compound 2, compound 14, or compound 19 (Plos ONE 7 (3): e32864, the contents of which are hereby incorporated by reference).

In various specific embodiments, the additional therapeutic agent is one or more of CKR-2B, a 2-thioimidazole, CCR2 Antagonist (CAS 445479-97-0), and CCX140.

In yet another embodiment, the additional therapeutic agent is a clodronate. In still another embodiment, the agent is a clodronate liposome preparation as described in Barrera et al. Arthritis and Rheumatism, 2000, 43, pp. 1951-1959, the contents of which are hereby incorporated by reference in their entireties.

In still another embodiment, the additional therapeutic agent is a chelated or unchelated form of gadolinium, for example gadolinium chloride (GAD).

In various embodiments, the additional therapeutic agent is a renin angiotensin system (RAS) inhibitor. In some embodiments, the renin angiotensin system (RAS) inhibitor is one or more of an angiotensin-converting enzyme (ACE) inhibitor, an angiotensin-receptor blocker, and a renin inhibitor.

Non limiting examples of angiotensin-receptor blockers which are useful in the present disclosure include, but are not limited to: irbesartan (U.S. Pat. No. 5,270,317, hereby incorporated by reference in its entirety), candesartan (U.S. Pat. Nos. 5,196,444 and 5,705,517 hereby incorporated by reference in their entirety), valsartan (U.S. Pat. No. 5,399,578, hereby incorporated by reference in its entirety), and losartan (U.S. Pat. No. 5,138,069, hereby incorporated by reference in its entirety).

In embodiments, the additional therapeutic agent is a nutraceutical agent. Non-limiting examples of nutraceutical agents include, Resveratrol, Taurine, carnosine, Quercetin, Curcumin, Glycyrrhizin, Naringin, Folate, Glutathione, N-acetylcysteine, Alpha Lipoic Acid, Quercetin, Zinc, Vitamin A, Vitamin C, Vitamin D, Vitamin K2, Vitamin B, Vitamin B6, Vitamin B9, Vitamin B12, Vitamin E, Magnesium, Bromelain, Cannabidiolic Acid, Cannabigerolic Acid, Cannabidiol, Cannabigerol, Cannabinol, NigeIIa Sativa/Thymoquinone, Selenium, Curcumin, Piperine, Astaxanthin, and/or Sulforaphane.

In embodiments, the additional therapeutic agent is a fatty acid agent. Non-limiting examples of fatty acid agents include polyunsaturated fatty acid (PUFA), monounsaturated fatty acid (MUFA), saturated fatty acid (SFA), Caprylic acid, Capric acid, Lauric acid, Myristic acid, Palmitic acid, Stearic acid, Arachidic acid, Behenic acid, Lignoceric acid, Cerotic acid, Myristoleic acid, Palmitoleic acid, Sapienc acid, Oleic acid, Elaidic acid, Vaccenic acid, Linoleic acid, Linoelaidic acid, a-Linolenic acid, Arachidonic acid, Eicosapentaenoic acid, Erucic acid, Docosahexaenoic acid, animal fats, and vegetable fats.

Further additional therapeutic agents, include, for example, one or more of anti-factor D antibody (e.g. lampalizumab (Genentech)), an anti-β-amyloid (anti-Aβ) antibody (e.g. GSK933776 (GSK), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), doxycycline (ORACEA), emixustat hydrochloride, sirolimus (RAPAMUNE), glatiramer acetate (COPAXONE) and an NSAID (e.g. ibuprofen, indomethacin). In some embodiments, one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (lic), formula (i2a), formula (i2b), formula (IIIa), formula (IIIb), formula (IV), formula (V), formula (VI), formula (VII), formula (VIIa), formula (VIIb), and formulas 1001 to 1005, or a pharmaceutically acceptable salt thereof is used adjunctively or neoadjunctively with these agents in the treatment of prevention of dry AMD and/or RPD.

Still further additional therapeutic agents, including in the context of dry AMD and RPD methods are found in Arq. Bras. Oftalmol, vol. 75 no. 1 January/February 2012, the entire contents of which are hereby incorporated by reference.

In other embodiments, the disclosure provides for the use of one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (lib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, or a pharmaceutically acceptable salt thereof, alone or in combination with an additional therapeutic agent in the manufacture of a medicament useful for the treatment or prevention of one or more ocular diseases or disorders.

### Definitions

The following definitions are used in connection with the disclosure disclosed herein. Unless defined otherwise, all technical and scientific terms used herein have the same meaning as commonly understood to one of skill in the art to which this disclosure belongs.

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

An “effective amount,” when used in connection with an agent effective for the treatment of an ocular disease or disorder, one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, or a pharmaceutically acceptable salt thereof, is an amount that is effective for treating or preventing an ocular disease or disorder such as, for example, a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; Wegener granulomatosis, optic neuritis, optionally wherein the optic neuritis is primary or secondary to systemic (i.e. non-ocular) disease, AMD and/or RPD.

An “effective amount,” when used in connection with another therapeutic agent is an amount that is effective for treating or preventing an ocular disease or disorder such as, for example, a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; Wegener granulomatosis, optic neuritis, optionally wherein the optic neuritis is primary or secondary to systemic (i.e. non-ocular) disease, AMD and/or RPD (e.g. providing a measurable treatment, prevention, or reduction in the rate of pathogenesis) alone or in combination with one or more of a compound of formula (I), formula (Ia), formula (Ib), formula (Ic), formula (Id), formula (Ie), formula (Ia1), formula (Ia2), formula (la3), formula (If), formula (Ig), formula (Ih), formula (Ii), formula (Ij), formula (Ik), formula (Im), formula (In), formula (lia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, or a pharmaceutically acceptable salt thereof. “In combination with” includes administration within the same composition and via separate compositions; in the latter instance, the other therapeutic agent is effective for treating or preventing a condition during a time when the agent a compound of one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (lic), formula (i2a), formula (i2b), formula (IIIa), formula (IIIb), formula (IV), formula (V), formula (VI), formula (VII), formula (VIIa), formula (VIIb), and formulas 1001 to 1005, or a pharmaceutically acceptable salt thereof, exerts its prophylactic or therapeutic effect, or vice versa.

An “effective amount,” when used in connection with a toxin, for example, sodium iodate, is an amount that is effective for inducing measurable atrophy of ocular tissue as described herein.

An agent is “useful for the treatment of an ocular disease or disorder” if the agent provides a measurable treatment, prevention, or reduction in the rate of pathogenesis of an ocular disease or disorder.

The term “ocular disease or disorder” refers to one of various ophthalmic diseases and includes diseases of the eye and the ocular adnexa. The term “ocular disease or disorder” includes, for example, disorders described herein (for instance, by way of non-limiting example, an ocular disease or disorder such as, for example, a diabetic eye disease (for instance, diabetic retinopathy and DME), Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; Wegener granulomatosis, optic neuritis, optionally wherein the optic neuritis is primary or secondary to systemic (i.e. non-ocular) disease, wet AMD, dry AMD, RPD, white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), diffuse subretinal fibrosis (DSF)), LORDs, and central serous retinopathy (CSR).

The term “neovascularization” refers to new blood vessel formation in abnormal tissue or in abnormal positions.

The term “VEGF” refers to a vascular endothelial growth factor that induces angiogenesis or an angiogenic process, including, but not limited to, increased permeability. As used herein, the term “VEGF” includes the various subtypes of VEGF (also known as vascular permeability factor (VPF) and VEGF-A) that arise by, e.g., alternative splicing of the VEGF-A/VPF gene including VEGF121, VEGF165 and VEGF 189. Further, as used herein, the term “VEGF” includes VEGF-related angiogenic factors such as PIGF (placental growth factor), VEGF-B, VEGF-C, VEGF-D and VEGF-E, which act through a cognate VEFG receptor (i.e., VEGFR) to induce angiogenesis or an angiogenic process. The term “VEGF” includes any member of the class of growth factors that binds to a VEGF receptor such as VEGFR-1 (FIt-1), VEGFR-2 (KDR/FIk-1), or VEGFR-3 (FLT-4). The term “VEGF” can be used to refer to a “VEGF” polypeptide or a “VEGF” encoding gene or nucleic acid.

The term “anti-VEGF agent” refers to an agent that reduces, or inhibits, either partially or fully, the activity or production of a VEGF. An anti-VEGF agent can directly or indirectly reduce or inhibit the activity or production of a specific VEGF such as VEGF 165. Furthermore, “anti-VEGF agents” include agents that act on either a VEGF ligand or its cognate receptor so as to reduce or inhibit a VEGF-associated receptor signal. Non-limiting examples of “anti-VEGF agents” include antisense molecules, ribozymes or RNAi that target a VEGF nucleic acid; anti-VEGF aptamers, anti-VEGF antibodies to VEGF itself or its receptor, or soluble VEGF receptor decoys that prevent binding of a VEGF to its cognate receptor; antisense molecules, ribozymes, or RNAi that target a cognate VEGF receptor (VEGFR) nucleic acid; anti-VEGFR aptamers or anti-VEGFR antibodies that bind to a cognate VEGFR receptor; and VEGFR tyrosine kinase inhibitors.

The term “anti-RAS agent” or “anti-Renin Angiotensin System agent” refers to refers to an agent that reduces, or inhibits, either partially or fully, the activity or production of a molecule of the renin angiotensin system (RAS). Non-limiting examples of “anti-RAS” or “anti-Renin Angiotensin System” molecules are one or more of an angiotensin-converting enzyme (ACE) inhibitor, an angiotensin-receptor blocker, and a renin inhibitor.

The term “steroid” refers to compounds belonging to or related to the following illustrative families of compounds: corticosteroids, mineralicosteroids, and sex steroids (including, for example, potentially androgenic or estrogenic or anti-andogenic and anti-estrogenic molecules). Included among these are, for example, prednisone, prednisolone, methyl-prednisolone, triamcinolone, fluocinolone, aldosterone, spironolactone, danazol (otherwise known as OPTINA), and others.

The terms “peroxisome proliferator-activated receptor gamma agent,” or “PPAR-γ agent,” or “PPARG agent,” or “PPAR-gamma agent” refers to agents which directly or indirectly act upon the peroxisome proliferator-activated receptor. This agent may also influence PPAR-alpha, “PPARA” activity.

The term “an agent that modulates autophagy” refers to a modulator of cell survival, cell death, survival, autophagy, proliferation, regeneration, and the like.

The term “monocyte chemotactic protein,” or “MCP” refers to the family or a member of the small inducible gene (SIG) family that plays a role in the recruitment of monocytes to sites of injury and infection. These terms can also refer to C-C motif chemokine molecules, whose activity include, for example, macrophage recruitment, and/or proliferation and/or activation. Non-limiting examples of MCPs include MCP-1, MCP-2, MCP-3, MCP-4, MCP-5, MCP-6, MCP-7, etc., and are also known as C-C motif chemokine ligand (ccl) ccl2, ccl3, ccl4, ccl5, ccl6, ccl7, etc.

“Alkyl” refers to a straight or branched hydrocarbon chain radical consisting solely of carbon and hydrogen atoms, containing no unsaturation, having from one to ten carbon atoms (e.g., (C1-10)alkyl or C1-10 alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O), R^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), or PO₃(R^{a}) 2 where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an-(alkyl) aryl radical where aryl and alkyl are as disclosed herein and which are optionally substituted by one or more of the substituents described as suitable substituents for aryl and alkyl respectively

“Alkylhetaryl” refers to an-(alkyl) hetaryl radical where hetaryl and alkyl are as disclosed herein and which are optionally substituted by one or more of the substituents described as suitable substituents for aryl and alkyl respectively.

“Alkylheterocycloalkyl” refers to an-(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkylheterocycloalkyl” refers to an-(alkyl) heterocyclyl radical where alkyl and heterocycloalkyl are as disclosed herein and which are optionally substituted by one or more of the substituents described as suitable substituents for heterocycloalkyl and alkyl respectively.

“Alkenyl” refers to a straight or branched hydrocarbon chain radical group consisting solely of carbon and hydrogen atoms, containing at least one double bond, and having from two to ten carbon atoms (i.e., (C2-10)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O) &R^{a} (where t is 1 or 2), —S(O)OR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an-(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C2-10)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an-(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀) cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a-(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a-(cycloalkyl) heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a-(cycloalkyl) heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group-O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O— (substituted alkyl). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O), R^{a} (where t is 1 or 2), —S(O)ₜOR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy) (C═O)-attached through the carbonyl carbon wherein the alkoxy group has the indicated number of carbon atoms. Thus a (C1-6)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, -N(R^{a})C(O)OR^{a}, —C(O)R^{a}, N(R^{a})₂, —C(O)SR^{a}, —OC(O)N(R^{a})₂, —C(O)N(R^{a})₂, —C(O)OR^{a}, N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R (C═O)O-radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, -OC(O)N(R^{a})₂, —C(O)N(R^{a})₂, —N(R^{a})C(O)OR^{a}, N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acylsulfonamide” refers a —S(O)₂—N(R^{a})—C(═O)— radical, where R^{a} is hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Unless stated otherwise specifically in the specification, an acylsulfonamide group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)OR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a-N(R^{a}) 2 radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a-N(R^{a}) 2 group has two R^{a} substituents other than hydrogen, they can be combined with the nitrogen atom to form a 4—, 5—, 6- or 7-membered ring. For example, —N(R^{a}) 2 is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{a}, and —NR^{a}R^{a} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4—, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)-R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “aryloxy” refers to the group-O-aryl.

The term “substituted aryloxy” refers to aryloxy wherein the aryl substituent is substituted (i.e., —O— (substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula-COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O), R^{a} (where t is 1 or 2), —S(O) &R^{a} (where t is 1 or 2), —S(O)ₜOR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an-(heteroalkyl) aryl radical where heteroalkyl and aryl are as disclosed herein and which are optionally substituted by one or more of the substituents described as suitable substituents for heteroalkyl and aryl, respectively.

“Heteroalkylheteroaryl” refers to an-(heteroalkyl) heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an-(heteroalkyl) heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an-(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” or “Het” refers to a 5- to 18-membered aromatic radical (e.g., C5-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta [4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7] cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —SC(O)—R^{a}, —-C(O)OR^{a}, N(R^{a})₂, —C(O)R^{a}, —C(O)OR^{a}, —C(O)SR^{a}, —OC(O)N(R^{a})₂, —C(O)N(R^{a})₂, N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(+)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or(S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or(S). The present chemical entities, pharmaceutical compositions and methods are meant to include all such possible isomers, including racemic mixtures, optically pure forms and intermediate mixtures. Optically active (R)- and(S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an(S)-isomeric configuration, is present as a racemic mixture, the enantiomeric purity is about 50% with respect to either the (R)- or(S)-isomer. If that compound has one isomeric form predominant over the other, for example, 80% (S)-isomer and 20% (R)-isomer, the enantiomeric purity of the compound with respect to the(S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the(S)-enantiomer, means a preparation of the compound having greater than 50% by weight of the(S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4 (1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S— (optionally substituted alkyl), —S— (optionally substituted aryl), —S— (optionally substituted heteroaryl) and —S— (optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)— (optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4—, 5—, 6- or 7-membered ring. A sulfonamido group is optionally substituted by one or more of the substituents described for alkyl, cycloalkyl, aryl, heteroaryl, respectively.

“Sulfoxyl” refers to a-S(═O) 2OH radical.

“Sulfonate” refers to a-S(═O) 2—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

As used herein, “a,” “an,” or “the” can mean one or more than one. Further, the term “about” when used in connection with a referenced numeric indication means the referenced numeric indication plus or minus up to 10% of that referenced numeric indication. For example, the language “about 50” covers the range of 45 to 55.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the disclosure, the present disclosure, or embodiments thereof, may alternatively be described using alternative terms such as “consisting of” or “consisting essentially of.”

### Pharmaceutically Acceptable Salts and Excipients

Any compound of formula (I), formula (Ia), formula (Ib), formula (Ic), formula (Id), formula (Ie), formula (la1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, a-hydroxybutyrate, butyne-1,4-dicarboxylate, hexyne-1,4-dicarboxylate, caprate, caprylate, cinnamate, glycoIIate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compounds of the present disclosure having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2—OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 is in the form or a pharmaceutically acceptable salt. In some embodiments, the pharmaceutically acceptable salt is a sodium salt. In some embodiments, an agent is in in the form of a pharmaceutically acceptable salt.

Further, any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In one aspect, the disclosure provides a pharmaceutical composition comprising two or more compounds of formula (I), formula (Ia), formula (Ib), formula (Ic), formula (Id), formula (Ie), formula (Ia1), formula (la2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, wherein at least one compound is a homodimer and at least one compound is a heterodimer. In some embodiments, the ratio of the homodimer to the heterodimer is selected from between about 1:99 to about 99:1. In some embodiments, the ratio of the homodimer to the heterodimer is selected from between about 1:50 to about 50:1. In some embodiments, the ratio of the homodimer to the heterodimer is selected from between about 1:25 to about 25:1. In some embodiments, the ratio of the homodimer to the heterodimer is selected from between about 1:10 to about 10:1. In some embodiments, the ratio of the homodimer to the heterodimer is selected from between about 1:5 to about 5:1. In some embodiments, the ratio of the homodimer to the heterodimer is selected from between about 1:3 to about 3:1. In some embodiments, the ratio of the homodimer to the heterodimer is selected from between about 1:2 to about 2:1. In some embodiments, the ratio of the homodimer to the heterodimer is about 1:1

In some embodiments, the pharmaceutical composition comprises one or more stabilizers. Non-limiting examples of stabilizers include hydroxypropylmethylcellulose, hydroxyethylcellulose, polyvinylpyrrolidone, carboxymethylcellulose (CMC), polyvinyl alcohol, sodium chondroitin sulfate, sodium hyaluronate, acacia (acacia), agar, aluminum magnesium silicate, sodium alginate, sodium stearate, bladder rack, bentonite, carbomer, carrageenan, carbo Paul, xanthan, cellulose, microcrystalline cellulose (MCC), seratonia, chitin, carboxymethylated chitosan, chondras, dextrose, furceIIaran, gelatin, Ghatti gum, guar gum, hectorite, lactose, sucrose, maltodextrin, mannitol, Sorbitol, honey, corn starch, wheat starch, rice starch, potato starch, gelatin, karaya gum, xanthan gum, tragaca Rubber, ethylcellulose, ethylhydroxyethylcellulose, ethylmethylcellulose, methylcellulose, hydroxyethylcellulose, hydroxyethylmethylcellulose, hydroxypropylcellulose, poly (hydroxyethylmethacrylate), oxypolygelatin, pectin, polygelin, povidone, propylene carbonate, methyl vinyl ether/maleic anhydride Copolymer (PVM/MA), poly(methoxyethyl methacrylate), poly(methoxyethoxyethyl methacrylate), hydroxypropylcellulose, hydroxypropylmethyl-cellulose (HPMC), sodium carboxymethyl-cellulose (CMC), silicon dioxide, polyvinylpyrrolidone (PVP: Povidone), Captisol, Splenda (registered trademark) (Dextrose, maltodextrin and sucralose) or combinations thereof. In some embodiments, the stabilizer is one or more selected from carboxymethylcellulose (CMC), Captisol, and Sorbitol.

### Formulations, Administration, and Dosing

Any compound of formula (I), formula (Ia), formula (Ib), formula (Ic), formula (Id), formula (Ie), formula (la1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIIa), formula (VIIb), and formulas 1001 to 1005 described herein can take the form of solutions, suspensions, emulsion, intra-ocular injection, intra-vitreal injection, topical ophthalmic drops, sub-conjunctival injection, sub-Tenon's injection, trans-scleral formulations, tablets, pills, pellets, capsules, capsules containing liquids, powders, sustained-release formulations, suppositories, emulsions, aerosols, sprays, suspensions, or any other form suitable for use. In one embodiment, the composition is suitable for an intra-vitreal injection (see, e.g., ILUVIEN or similar forms) or implantation (see, e.g., RETISERT or similar forms). In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In one embodiment, any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein is formulated for ophthalmic administration, including, for example, intravitreal or intraocular administration, topical, and/or intravenous administration. Typically, compositions for administration comprise sterile isotonic aqueous buffer. Where necessary, the compositions can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as lignocaine to lessen pain at the site of the injection.

In one embodiment, any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein is formulated in accordance with routine procedures as a composition adapted for intra-ocular administration.

In some embodiments, the pharmaceutical composition is formulated for extended release. In some embodiments, the pharmaceutical composition is formulated for ocular delivery.

In one embodiment, any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein is formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any agent described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade.

The ingredients may be supplied either separately or mixed together in unit dosage form, for example, as a pre-mixed solution, dry lyophilized-powder, or water-free concentrate in a hermetically sealed container such as an ampule, pre-filled syringe, or sachets indicating the quantity of active agent. Where any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIIa), formula (VIIb), and formulas 1001 to 1005 described herein is to be administered by intra-vitreal or intra-ocular delivery, it can be dispensed, for example, with a pre-filled syringe or injector, or in an ampule for withdrawal into a suitable syringe. Where any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein is to be administered by infusion, it can be dispensed, for example, with an infusion bottle containing sterile pharmaceutical grade water or saline. Where any compound of formula (I), formula (Ia), formula (Ib), formula (Ic), formula (Id), formula (Ie), formula (Ia1), formula (Ia2), formula (la3), formula (If), formula (Ig), formula (Ih), formula (Ii), formula (Ij), formula (Ik), formula (Im), formula (In), formula (lia), formula (Iib), formula (Iic), formula (i2a), formula (i2b), formula (IIIa), formula (IIIb), formula (IV), formula (V), formula (VI), formula (VII), formula (VIIa), formula (VIIb), and formulas 1001 to 1005 described herein is to be administered by injection, an ampule of sterile water for injection or saline can be provided so that the ingredients can be mixed prior to administration.

Any compound of formula (I), formula (Ia), formula (Ib), formula (Ic), formula (Id), formula (Ie), formula (la1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598, 123; 4,008,719; 5,674,533; 5,059,595; 5,591,767; 5, 120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, nanoparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The disclosure thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

Compositions can be prepared according to conventional mixing, granulating, coating or polymerization methods, respectively, and the present compositions can comprise, in one embodiment, from about 0.1% to about 99%; and in another embodiment from about 1% to about 70% of any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (lic), formula (i2a), formula (i2b), formula (IIIa), formula (IIIb), formula (IV), formula (V), formula (VI), formula (VII), formula (VIIa), formula (VIIb), and formulas 1001 to 1005 described herein by weight or volume.

In another embodiment, any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein acts synergistically when co-administered with another agent and is administered at doses that are lower than the doses commonly employed when such agents are used as monotherapy.

For example, the dosage of any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein as well as the dosing schedule can depend on various parameters, including, but not limited to, the ocular disease or disorder being treated, the subject's general health, and the administering physician's discretion. Any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional therapeutic agent, to a subject in need thereof. In various embodiments any compound of formula (I), formula (Ia), formula (Ib), formula (Ic), formula (Id), formula (Ie), formula (Ia1), formula (la2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIIa), formula (VIIb), and formulas 1001 to 1005 described herein is administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, or their pharmaceutically acceptable salts, and one or more additional therapeutic agents are administered within 3 hours. In another embodiment, one or more of a compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (lic), formula (i2a), formula (i2b), formula (IIIa), formula (IIIb), formula (IV), formula (V), formula (VI), formula (VII), formula (VIIa), formula (VIIb), and formulas 1001 to 1005, or their pharmaceutically acceptable salts, and one or more additional therapeutic agents are administered at 1 minute to 24 hours apart.

The amount of any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIIa), formula (VIIb), and formulas 1001 to 1005 described herein that is admixed with the carrier materials to produce a single dosage can vary depending upon the subject being treated and the particular mode of administration. In vitro or in vivo assays can be employed to help identify optimal dosage ranges.

In general, the doses that are useful are known to those in the art. For example, doses may be determined with reference Physicians' Desk Reference, 66th Edition, PDR Network; 2012 Edition (Dec. 27, 2011), the contents of which are incorporated by reference in its entirety.

The dosage of any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIIa), formula (VIIb), and formulas 1001 to 1005 described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular ocular disease or disorder being treated, the severity of the disorder, and the anatomical location of the disorder. Some variations in the dosage can be expected.

In some embodiments, the administering is effected orally or intra-vascularly, or intraocularly, or periocularly, or to the ocular surface

When ophthalmically administered to a human, for example, intravitreally, the dosage of one or more of a compound of formula (I), formula (Ia), formula (Ib), formula (Ic), formula (Id), formula (Ie), formula (Ia1), formula (la2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, and/or additional therapeutic agent is normally 0.003 mg to 5.0 mg per eye per administration, or 0.03 mg to 3.0 mg per eye per administration, or 0.1 mg to 1.0 mg per eye per administration. In one embodiment, the dosage is 0.03 mg, 0.3 mg, 1.5 mg or 3.0 mg per eye. In another embodiment, the dosage is 0.5 mg per eye. The dosage can range from 0.01 mL to 0.2 mL administered per eye, or 0.03 mL to 0.15 mL administered per eye, or 0.05 mL to 0.10 mL administered per eye. In one embodiment, the administration is 400 μg of compound, monthly for at least three months.

Generally, when orally administered to a mammal, the dosage of any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIIa), formula (VIIb), and formulas 1001 to 1005, described herein may be 0.001 mg/kg/day to 100 mg/kg/day, 0.01 mg/kg/day to 50 mg/kg/day, or 0.1 mg/kg/day to 10 mg/kg/day. When orally administered to a human, the dosage of any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, described herein is normally 0.001 mg to 1000 mg per day, 1 mg to 600 mg per day, or 5 mg to 30 mg per day. In one embodiment, oral dosage is 600 mg per day. In one embodiment, the oral dosage is two 300 mg doses per day. In another embodiment, oral dosage is 7.5 mg per week to 15 mg per week.

For administration of any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, described herein by parenteral injection, the dosage is normally 0.1 mg to 250 mg per day, 1 mg to 20 mg per day, or 3 mg to 5 mg per day. Injections may be given up to four times daily. Generally, when orally or parenterally administered, the dosage of any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein is normally 0.1 mg to 1500 mg per day, or 0.5 mg to 10 mg per day, or 0.5 mg to 5 mg per day. A dosage of up to 3000 mg per day can be administered.

In some embodiments, it may be desirable to administer one or more of any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (lib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein to the eye. Administration may be, by way of non-limiting example, intra-ocular, intra-vitreal, topical (including, but not limited to, drops and ointment), sub-conjunctival, sub-Tenon's, trans-scleral, suprachoroidal, subretinal, and via iontophoresis.

Other routes of administration may also be used, such as, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin.

The mode of administration can be left to the discretion of the practitioner, and depends in-part upon the site of the medical condition. In most instances, administration results in the release of any agent described herein into the bloodstream.

Any compound of formula (I), formula (Ia), formula (Ib), formula (Ic), formula (Id), formula (Ie), formula (la1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein can be administered orally. Such compounds can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

Further methods of administration include but are not limited to intra-ocular, intra-vitreal, topical ocular (including but not limited to drops, ointments and inserts), sub-conjunctival, sub-Tenon's, suprachoroidal, trans-scleral, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In some embodiments, more than one of any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (lic), formula (i2a), formula (i2b), formula (IIIa), formula (IIIb), formula (IV), formula (V), formula (VI), formula (VII), formula (VIIa), formula (VIIb), and formulas 1001 to 1005 described herein is administered to the eye. Administration may be, by way of non-limiting example, intra-ocular, intra-vitreal, topical (including, but not limited to, drops and ointment), sub-conjunctival, sub-Tenon's, trans-scleral, and iontophoresis. The mode of administration can be left to the discretion of the practitioner, and depends in-part upon the site of the medical condition. In most instances, administration results in the release into the bloodstream.

In some embodiments, bindarit or an analogue thereof and/or a bindarit ethyl ester or an analogue thereof is co-administered in conjunction with an anti-vascular endothelial growth factor (VEGF) agent, a modulator of the complement cascade, a non-steroidal anti-inflammatory drug (NSAID), an angiotensin-converting enzyme (ACE) inhibitor, a peroxisome proliferator-activated receptor (PPAR) modulator, a renin inhibitor, a corticosteroid, an antibody, a soluble receptor, a biological molecule, a nuclear receptor protein (e.g. LXR), a Liver X receptor (LXR) agonist, a steroid (e.g. fluocinolone acetonide, dexamethasone), a fatty acid mimtic (FAM) molecule, or an agent that modulates autophagy. In some embodiments, bindarit or an analogue thereof and/or bindarit ethyl ester or an analogue thereof is co-administered in conjunction with an NSAID. In some embodiments, the NSAID is selected from indomethacin (Indocin), ibuprofen, diclofenac (Voltaren), acemetacin, diclofenac, naproxen, ketorolac, carprofen, etodolac, fentiazac, flurbiprofen, ketoprofen, oxaprozin, pranoprofen, sulindac, suxibuzone, celecoxib (Celebrex®), fenoprofin (Nalfon), ketorolac tromethamine, tolmetin, diflunisal, meclofenamate sodium, mefenamic acid, etoricoxib, meloxicam, nabumetone, piroxicam, or derivatives thereof. In some embodiments, bindarit or an analogue thereof and/or bindarit ethyl ester or an analogue thereof is co-administered in conjunction with indomethacin (Indocin). In some embodiments, bindarit or an analogue thereof and/or bindarit ethyl ester or an analogue thereof is co-administered in conjunction with ibuprofen. Co-administration can be simultaneous or sequential. The term “simultaneously” as used herein, includes a time separation of no more than about 60 minutes, such as no more than about 30 minutes, no more than about 20 minutes, no more than about 10 minutes, no more than about 5 minutes, or no more than about 1 minute. The term “sequentially” as used herein includes a time separation of more than about 60 minutes. For example, the time between the sequential administration can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or pharmacodynamic activity. Non-limiting examples of co-administration include administration by a single syringe, separate syringes, topical drops, and slow-release implants. In a non-limiting example, bindarit or an analogue thereof and/or a bindarit ethyl ester or an analogue thereof is co-administered in conjunction with an NSAID by a single syringe, separate syringes, topical drops, and slow-release implants.

In specific embodiments, it may be desirable to administer locally to the area in need of treatment.

In another embodiment, delivery can be in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989). In yet another embodiment, delivery can be in a controlled release system. In one embodiment, a slow release intra-ocular device may be used. In some embodiments, this device consists of a locally delivered erodible or non-erodable liquid, gel, polymer, etc.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In another embodiment, a controlled-release system can be placed in proximity of the target area to be treated, e.g., the retina, thus requiring only a fraction of the systemic dose (see, e.g., Goodson, in Medical Applications of Controlled Release, supra, vol. 2, pp. 115-138 (1984). Other controlled-release systems discussed in the review by Langer, 1990, Science 249:1527-1533) may be used.

Administration of any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein can, independently, be one to four times daily or one to four times per month or one to six times per year or once every two, three, four or five years. Administration can be for the duration of one day or one month, two months, three months, six months, one year, two years, three years, and may even be for the life of the subject. Chronic, long-term administration will be indicated in many cases. The dosage may be administered as a single dose or divided into multiple doses. In general, the desired dosage should be administered at set intervals for a prolonged period, usually at least over several weeks or months, although longer periods of administration of several months or years or more may be needed.

The dosage regimen utilizing any agent described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed. Any compound of formula (I), formula (Ia), formula (Ib), formula (Ic), formula (Id), formula (Ie), formula (Ia1), formula (Ia2), formula (Ia3), formula (If), formula (Ig), formula (Ih), formula (Ii), formula (Ij), formula (Ik), formula (Im), formula (In), formula (Iia), formula (Iib), formula (lic), formula (i2a), formula (i2b), formula (IIIa), formula (IIIb), formula (IV), formula (V), formula (VI), formula (VII), formula (VIIa), formula (VIIb), and formulas 1001 to 1005 described herein can be administered in a single daily dose, or the total daily dosage can be administered in divided doses of two, three or four times daily. Furthermore, any compound of formula (I), formula (Ia), formula (Ib), formula (Ic), formula (Id), formula (Ie), formula (Ia1), formula (la2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005 described herein can be administered continuously rather than intermittently throughout the dosage regimen.

Subjects and/or Animals

In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In some embodiments, the subject and/or animal may comprise fluorescently-tagged cells (with e.g. GFP). In some embodiments, the subject and/or animal is a transgenic animal comprising a fluorescent cell, such as, for example, an RPE cell and/or an immune cell. In some embodiments, the subject and/or animal is a human. In some embodiments, the human is a pediatric human. In other embodiments, the human is an adult human. In other embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the disclosure pertains to veterinary use. In a specific embodiment, the non-human animal is a household pet. In another specific embodiment, the non-human animal is a livestock animal.

In various embodiments, a subject's and/or an animal's eye comprises (i) a fluorescent compound in an amount effective to indicate the presence of an ocular disease or disorder in the subject and/or animal and (ii) a toxin in an amount effective to induce atrophy of ocular tissue. In some embodiments, such a subject and/or animal is administered an agent of the disclosure or is not administered an agent of the disclosure.

In various embodiments, RPE and immune cells are evaluated and/or effected. In some embodiments, immune cells include cells of a subject's and/or animal's innate immune system. In some embodiments, such cells include, but are not limited to, macrophage, monocyte, and microglial cells. In various embodiments, the disclosure provides for detecting a presence, detecting an absence, or measuring an amount of immune cells in a subject's and/or animal's eye

### Kits

The disclosure provides kits that can simplify the administration of any agent described herein. An exemplary kit of the disclosure comprises any agent described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the ocular surface. The kit can also further comprise one or more additional agent described herein.

In one embodiment, the kit comprises a container containing an effective amount of one or more of a compound of formula (I), formula (Ia), formula (Ib), formula (Ic), formula (Id), formula (Ie), formula (Ia1), formula (la2), formula (Ia3), formula (If), formula (Ig), formula (Ih), formula (Ii), formula (Ij), formula (Ik), formula (Im), formula (In), formula (Iia), formula (Iib), formula (Iic), formula (i2a), formula (i2b), formula (IIIa), formula (IIIb), formula (IV), formula (V), formula (VI), formula (VII), formula (VIla), formula (VIIb), and formulas 1001 to 1005, or a pharmaceutically acceptable salt thereof, and an effective amount of another therapeutic agent, such those described herein.

The invention will be further described in the following examples, which do not limit the scope of the invention described in the claims.

### EXAMPLES

### Example 1: In Vitro Studies Demonstrating Efficacy of Bindarit Ethyl Ester in Human Macrophages

This Example describes in vitro studies demonstrating the efficacy of bindarit ethyl ester in human macrophages.

Human THP-1 monocytes were seeded at 5×10⁵-9×10⁵ in a 6-well plate. Phorbol myristate acetate (5 ng/ml, PMA) was added for 48-72 hours, to induce differentiation from monocytes to macrophages. Cells were then starved for 3 hours. Lipopolysaccharide (LPS) was then added at 100 ng/ml for 1 hour, with or without bindarit or the bindarit ethyl ester. Cell were simultaneously exposed to 150 μM of bindarit or 150 μM of ethyl ester.

Cells were harvested in RNALater (Qiagen), and prepared for quantitative polymerase chain reaction (qPCR). Total RNA was isolated using Trizol Reagent (Life Technologies) based on the manufacturer's protocol and its quantity and quality checked with a Nanodrop 2000 spectrophotometer (Thermo Scientific, USA). QuantiTect Reverse Transcription Kit with separate genomic DNA removal step (Qiagen) was used based on the manufacturer's protocol. Briefly, 0.5 μg total RNA was used for each sample during reverse transcription and the resulting cDNA solution was further diluted to 10 ng/ul with nuclease free water and stored at-20 C.

PowerUp SYBR Green Master Mix (ThermoFisher Scientific) was used for quantitative PCR based on manufacturer's protocol. Briefly, 10 ng cDNA was used for each sample and the forward and reverse primer concentration was 2 UM each. PCR solution was subjected to 50° C. for 2 min, 95 C for 2 min, followed by 40 cycles of 95 C for 15 sec and 60° C. for 1 min. Standard melting curve analysis was also performed. The reaction was carried on qTower384G (analytik jena) 384-well PCR machine.

Relative quantification method based on delta delta Ct was used for comparison of gene expression levels in various arms. Briefly, relative quantification and statistical analysis were performed using Relative Expression Software Tool-Multiple Condition Solver (REST-MCS, V2) based on the protocol by M. W. Pfaffl & G. W. Horgan (2006), which is incorporated by reference herein in its entirety.

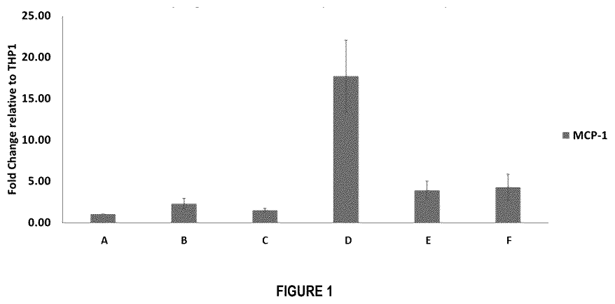

In particular, as monocyte chemoattractant protein-1 (MCP-1) is a primary target of bindarit and bindarit ethyl ester, a comparison of their ability to reduce MCP-1 mRNA is shown in FIG. 1, demonstrating that bindarit ethyl ester is equally as efficacious as bindarit.

### Example 2: Characterization Data of Bindarit-Resveratrol Conjugate

This Example describes characterization data for bindarit-resveratrol conjugate (formula (V)).

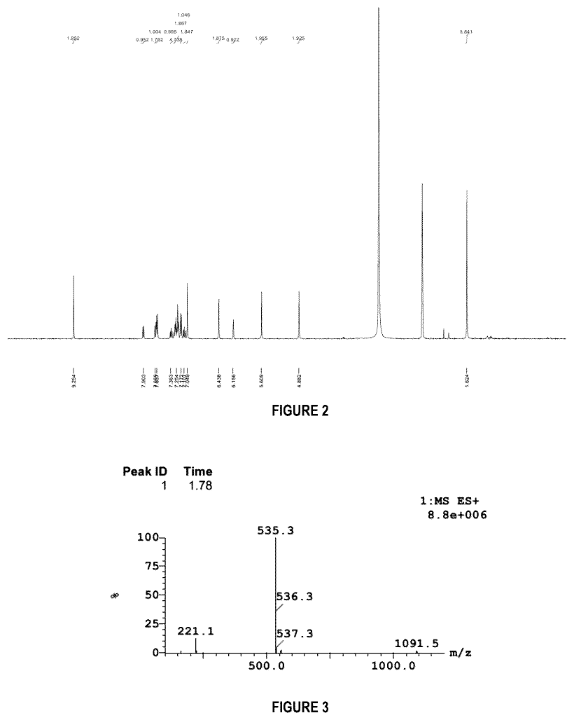

¹H NMR (DMSO-d₆, 400 MHZ): dH 9.25 (2H, s), 7.90 (1H, d, J=8.1 Hz), 7.67 (1H, d, J=8.6 Hz), 7.64 (2H, d, J=8.2 Hz), 7.36 (1H, t, J=7.7 Hz), 7.21-7.28 (5H, m), 7.17 (2H, d, J=8.3 Hz), 7.11 (1H, t, J=7.5 Hz), 7.05 (2H, s), 6.44 (2H, d, J=2.1 Hz), 6.16 (1H, s), 5.61 (2H, s), 4.88 (2H, s), 1.62 (6H, s). See FIG. 2 for NMR spectrum.

HRMS (ES+) m/z 535.3 [(M+H+); calculated for C₃₂H₃₁N₂O₅+H 535.2]. See FIG. 3 for spectrum.

### Example 3: Synthesis and Characterization of Bindarit Conjugates

This Example describes the synthesis and characterization of bindarit-taurine conjugate (formula (VI) and bindarit-carnosine conjugate (formula (VI)).

Bindarit was successfully coupled with taurine and carnosine, and purified by normal phase flash chromatography. Formation of triethylamine salt was observed when dichloromethane (DCM) was used as the reaction solvent for the bindarit conjugation to taurine. Triethylamine salt was isolated and removed by washing. Removal of the salt is helpful to avoid formation of emulsions, which contributes to a poorer overall yield of the conjugate and affects scalability of the synthesis. Use of tetrahydrofuran instead of DCM was found to eliminate the formation of the triethylamine salt, and is scalable.

Hydrolysis experiments were completed with 12M HCl and digestive enzymes, demonstrating that the bindarit conjugates are capable of cleavage under acidic and enzymatic conditions. Both conjugates showed hydrolysis in 12M HCl at 40° C. Taurine conjugate hydrolyzed at a greater rate, achieving 70% hydrolysis overnight compared to 50% for carnosine. Enzyme hydrolysis was also found to be successful at 17% conversion, using an esterase. N-acyl taurines are easily hydrolysed by FAAH (Fatty acid amide hydrolase), which are also readily expressed in the retina.

Synthesis of Bindarit N-hydroxysuccinimide (NHS) ester in dichloromethane-Synthesis 1

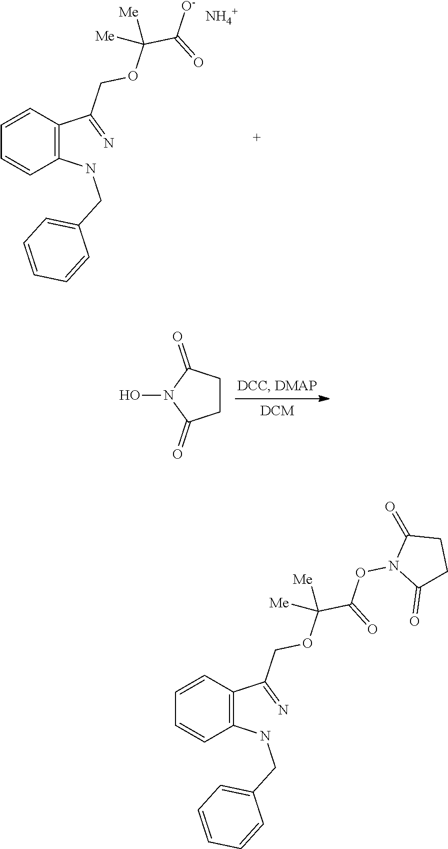 [SMILES: CC(C)(OCC(=N)c1ccccc1NCc1ccccc1)C(=O)[O-] ; [NH4+] ; O=C1CCC(=O)N1O ; CC(C)(OCC(=N)c1ccccc1NCc1ccccc1)C(=O)ON1C(=O)CCC1=O]

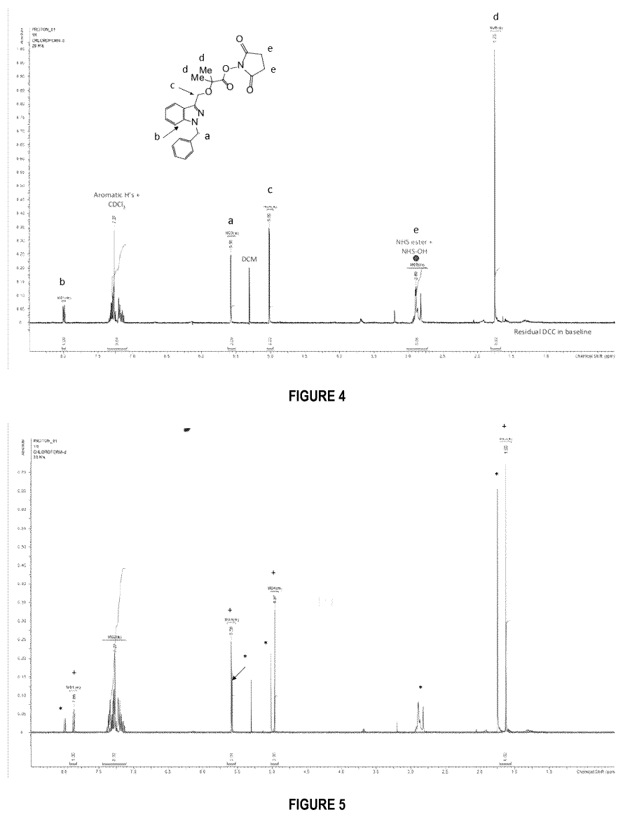

2.00 g (1.0 mol equiv.) of bindarit·NH4⁺ was dissolved in 40 ml dichloromethane (DCM) at ambient conditions. 2.71 g of N,N′-dicyclohexylcarbodiimide (DCC) (2.2 mol. equiv.) and 0.036 g dimethylaminopyridine (DMAP) (0.005 mol equiv.) were added to the reaction mixture. 2.75 g N-hydroxysuccinimide (NHS-OH) (4.1 mol equiv.) was added, and the reaction mixture stirred overnight (16 hours). IPC NMR indicated the reaction was completed after <30 min. The solids were removed by filtration, and the resulting filtrate was concentrated to provide 3.28 g (133%) crude bindarit·NHS ester. FIG. 4 is a ¹H NMR spectrum of bindarit·NHS ester in CDCl3. Several chemical shifts were observed when the bindarit·NHS ester ¹H NMR spectrum was compared to the bindarit ¹H NMR spectrum, including the shift of protons b from 7.85 ppm to 7.99 ppm, protons c shifted from 4.97 ppm to 5.02 ppm, and protons d shifted from 1.63 ppm to 1.75 ppm. FIG. 5 illustrates an overlay of the bindarit 1H NMR spectrum and the bindarit·NHS ester ¹H NMR spectrum.

### Synthesis of Bindarit-Taurine Conjugate—Synthesis 1

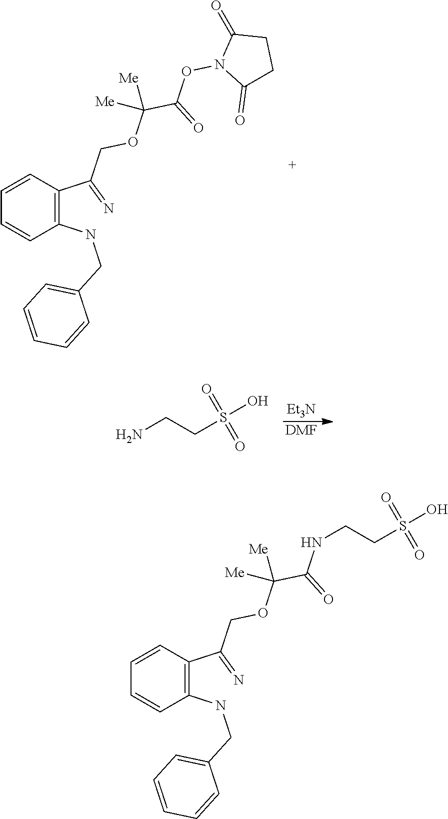

985 mg of bindarit·NHS ester (1.0 mol equiv.) was dissolved in 50 ml dimethylformamide (DMF) at ambient conditions. The reaction mixture was charged with 1.66 mL triethylamine (Et₃N) (5.1 mol equiv.). 672 mg taurine (2.3 mol equiv.) was added, and the reaction mixture was stirred overnight (22 hours). Solids were removed by filtration, and the resulting filtrate was concentrated to provide 1.68 g (167%) crude bindarit-taurine conjugate.

Crude bindarit-taurine conjugate was purified using flash chromatography (Biotage Isolera One). About 500 mg crude bindarit-taurine conjugate was loaded onto a 10 g sfar normal phase 60 μm silica column using 1 mL DCM. Impurities were eluted using a DCM: methanol solvent system as follows: 3 CV (95:5); 5 CV (95:5 to 80:20), 2 CV (80:20). Pure bindarit-taurine conjugate was eluted using a DCM: acetone solvent system as follows: 3 CV (100:0); 5 CV (90:10), 9 CV (75:25). Yield: 206 mg (41%) bindarit-taurine conjugate. Product collected was triethylamine salt form.

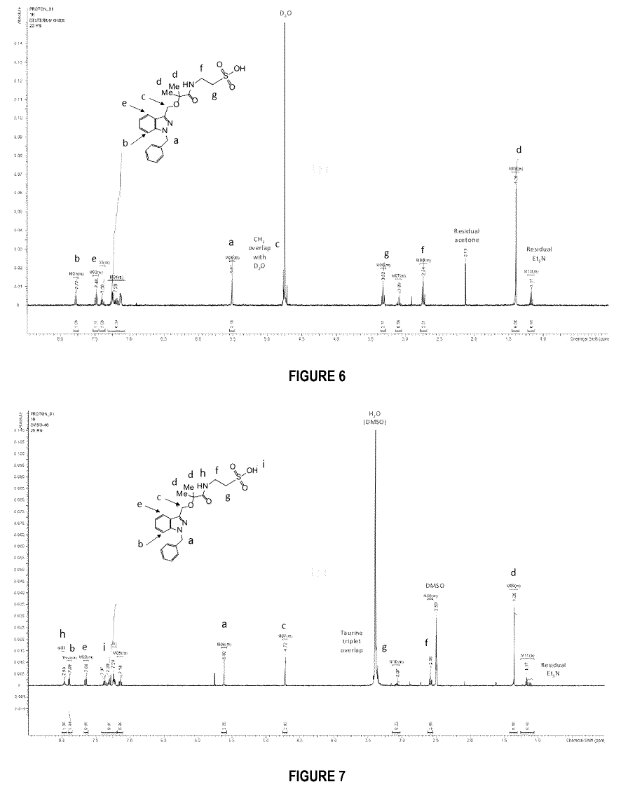

Triethylamine salt was removed as follows: 200 mg of bindarit-taurine triethylamine salt was dissolved in 4 mL DCM. The DCM layer was washed with 2×2 mL 1 M HCl and 1×2 mL brine. An emulsion was formed with each wash. The organic layer was dried with sodium sulfate and filtered, and the resulting filtrate was concentrated. Yield: 51 mg (31%) bindarit-taurine conjugate (2.4% triethylamine remaining-% w/w-NMR estimate). Overall yield: 23%. FIG. 6 is an image of a ¹H NMR spectrum of the bindarit-taurine conjugate (formula (VI)) in D2O. FIG. 7 is an image of a ¹H NMR spectrum of the bindarit-taurine conjugate (formula (VI) in DMSO.

### Synthesis of Bindarit N-Hydroxysuccinimide (NHS) Ester in Tetrahydrofuran—Synthesis 2

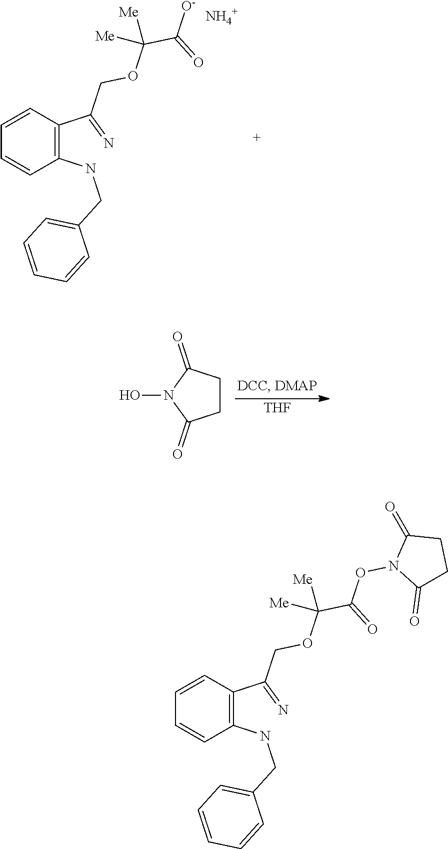 [SMILES: CC(C)(OCC(=N)c1ccccc1NCc1ccccc1)C(=O)[O-] ; [NH4+] ; O=C1CCC(=O)N1O ; CC(C)(OCC(=N)c1ccccc1NCc1ccccc1)C(=O)ON1C(=O)CCC1=O ; "THF"]

2.00 g scale

2.00 g (1.0 mol equiv.) of bindarit·NH4+ was dissolved in 40 mL tetrahydrofuran (THF) at ambient conditions. 2.42 g of N,N′-dicyclohexylcarbodiimide (DCC) (2.0 mol. equiv.) and 0.034 g dimethylaminopyridine (DMAP) (0.005 mol equiv.) were added to the reaction mixture. 2.71 g N-hydroxysuccinimide (NHS-OH) (4.1 mol equiv.) was added, and the reaction mixture stirred for 2.5 hours. Solids were removed by filtration to provide 35 mL crude bindarit·NHS ester.

### Synthesis of Bindarit-Taurine Conjugate—Synthesis 2

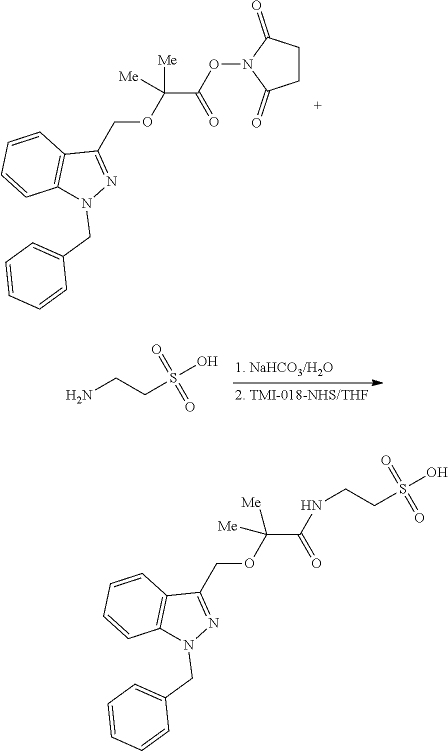 [SMILES: CC(C)(OCc1nn(Cc2ccccc2)c2ccccc12)C(=O)ON1C(=O)CCC1=O ; NCCS(=O)(=O)O ; CC(C)(OCc1nn(Cc2ccccc2)c2ccccc12)C(=O)NCCS(=O)(=O)O]

Scale: 301 mg taurine

301 mg taurine (2.0 mol equiv.) and 221 mg NaHCO₃ (2.2. mol equiv.) were dissolved in 10 mL high purity water (HPW) at ambient conditions. The reaction mixture was cooled in an ice bath for 20 minutes, followed by the addition of 5.3 mL of a bindarit·NHS ester/THE solution dropwise over 26 minutes. The ice bath was removed, and the reaction mixture was stirred overnight (~21 hours). The reaction mixture was concentrated to remove about 5 mL THF, and then filtered. The filtrate was diluted with 10 mL HPW and 12 mL of 1 M HCl was added dropwise, stirred for 30 min (no filterable solids), and concentrated to dryness. Yield: 1.1 g crude bindarit-taurine conjugate.

Crude bindarit-taurine conjugate was purified using flash chromatography (Biotage Isolera One). 1.1 g crude bindarit-taurine conjugate was loaded onto a 25 g sfar normal phase 60 μm silica column using 5 mL DCM: MeOH (75:25) via syringe. Pure bindarit-taurine conjugate was eluted using a DCM: MeOH solvent system as follows: 5 CV (95:5); 6.5 CV (95:5 to 70:30). Yield: 251 mg bindarit-taurine conjugate (8.4% MeOH remaining (% w/w-NMR estimate). Overall yield 63%.

Scale: 3.03 g taurine

4.02 g (1.0 mol equiv.) of bindarit·NH4+ was dissolved in 80 mL tetrahydrofuran (THF) at ambient conditions. 4.85 g of N,N′-dicyclohexylcarbodiimide (DCC) (2.0 mol. equiv.) and 0.077 g dimethylaminopyridine (DMAP) (0.005 mol equiv.) were added to the reaction mixture. 5.54 g N-hydroxysuccinimide (NHS—OH) (4.1 mol equiv.) was added, and the reaction mixture stirred for 4 hours. Reaction was determined to be completed by NMR. Solids were removed by filtration to provide crude bindarit·NHS ester, which was carried forward to taurine reaction.

3.03 g taurine (2.0 mol equiv.) and 2.02 g NaHCO₃ (2.0 mol equiv.) were dissolved in 140 mL high purity water (HPW) at ambient conditions. The reaction mixture was cooled in an ice bath for 20 minutes, followed by the addition of about 80 mL of a bindarit·NHS ester/THF solution dropwise over 32 minutes. The ice bath was removed, and the reaction mixture was stirred overnight (18 hours). The reaction mixture was concentrated to remove about 80 mL THF, and then filtered. 48 mL 1 M HCl (4.0 mol equiv.) was added dropwise, the reaction mixture stirred for 10 minutes (no filterable solids), and concentrated to dryness. Yield: 10.32 g crude bindarit-taurine conjugate (FIG. 13).

Crude bindarit-taurine conjugate was purified using flash chromatography (Biotage Isolera One). ~ 7 g crude bindarit-taurine conjugate was loaded onto a 100 g sfar normal phase 20 jum silica column using 40 ml DCM: MeOH (75:25) via syringe (solubility was an issue; about 2.98 crude bindarit-taurine conjugate remained in flask). Pure bindarit-taurine conjugate was eluted using a DCM: MeOH solvent system as follows: 5 CV (95:5); 3.5 CV (95:5 to 82:18). Yield: 1.63 g bindarit-taurine conjugate (~1.0% DCM remaining (% w/w-NMR estimate). Overall yield 32%.

### Synthesis of Bindarit-Carnosine Conjugate

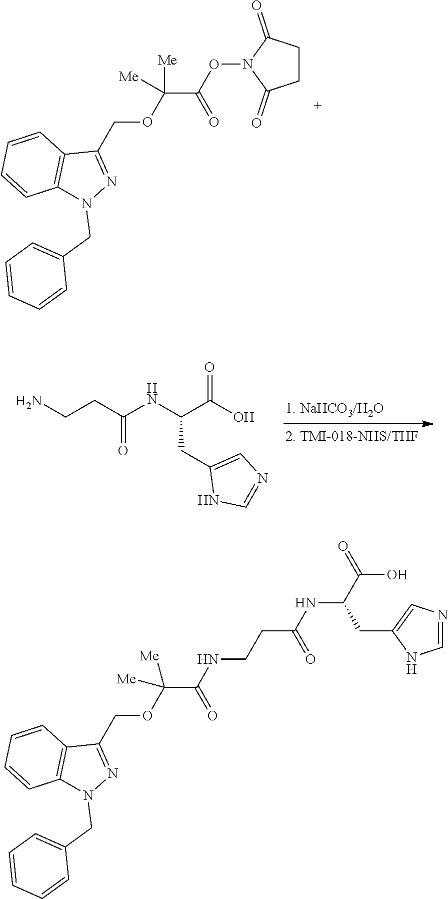 [SMILES: CC(C)(OCc1nn(Cc2ccccc2)c2ccccc12)C(=O)ON1C(=O)CCC1=O ; NCCC(=O)N[C@@H](Cc1cnc[nH]1)C(=O)O ; CC(C)(OCc1nn(Cc2ccccc2)c2ccccc12)C(=O)NCCC(=O)N[C@@H](Cc1cnc[nH]1)C(=O)O]

### Scale: 566 mg L-carnosine

566 mg L-carnosine (2.1 mol equiv.) and 222 mg Na CO3 (2.2 mol equiv.) were dissolved in 10 mL high purity water (HPW) at ambient conditions. The reaction mixture was cooled in an ice bath for 20 minutes, followed by the addition of 5.5 mL of a bindarit·NHS ester/THF solution (1.0 mol equiv.) dropwise over 10 minutes. The ice bath was removed, and the reaction mixture was stirred overnight (17 hours). The reaction mixture was concentrated to remove about 5 mL THF, and then filtered. The filtrate was diluted with 10 mL HPW and 12 mL of 1 M HCl was added dropwise, stirred for 30 min (no filterable solids), and concentrated to dryness. Yield: 1.160 g crude bindarit-carnosine conjugate.

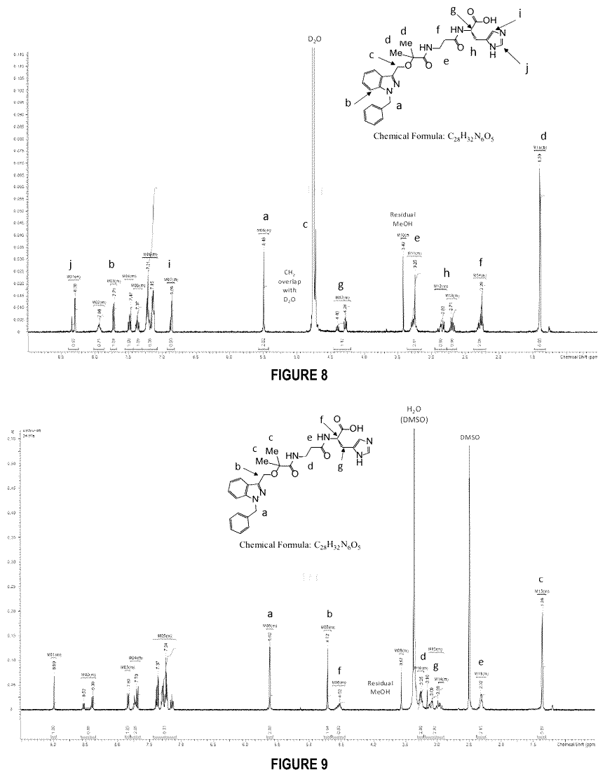

Crude bindarit-carnosine conjugate was purified using flash chromatography (Biotage Isolera One). 1.1 g crude bindarit-carnosine conjugate was loaded onto a 25 g sfar normal phase 60 μm silica column using 5 mL DCM: MeOH (75:25) via syringe. Pure bindarit-carnosine conjugate was eluted using a DCM: MeOH solvent system as follows: 3 CV (95:5); 10 CV (95:5 to 60:40), 5 CV (60:40 to 20:80), 2 CV (20:80). Yield: 202 mg bindarit-carnosine conjugate (2.0% MeOH remaining (% w/w-NMR estimate). Overall yield 42%. FIG. 8 is an image of a ¹H NMR spectrum of the bindarit-carnosine conjugate (formula (VIIa)) in D2O. FIG. 9 is an image of a 1H NMR spectrum of the bindarit-carnosine conjugate (formula (VIla)) in DMSO.

### Scale: 9.99 g L-carnosine

6.00 g (1.0 mol equiv.) of bindarit NH4+ was dissolved in 120 mL tetrahydrofuran (THF) at ambient conditions. 7.32 g of N,N′-dicyclohexylcarbodiimide (DCC) (2.0 mol. equiv.) and 0.112 g dimethylaminopyridine (DMAP) (0.005 mol equiv.) were added to the reaction mixture. 8.98 g N-hydroxysuccinimide (NHS—OH) (4.4 mol equiv.) was added, and the reaction mixture stirred for 6 hours. Reaction was determined to be completed by NMR. Solids were removed by filtration to provide crude bindarit·NHS ester, which was carried forward to carnosine reaction.

9.99 mg carnosine (2.5 mol equiv.) and 3.21 g Na CO3 (2.2 mol equiv.) were dissolved in 200 mL high purity water (HPW) at ambient conditions. The reaction mixture was cooled in an ice bath for 17 minutes, followed by the addition of ~120 mL of a bindarit·NHS ester/THF solution (1.0 mol equiv.) dropwise over 20 minutes. The ice bath was removed, and the reaction mixture was stirred overnight (18 hours). The reaction mixture was checked by NMR. The reaction mixture was concentrated to remove about 120 mL THF, and then filtered. 200 ml of 1 M HCl (11.9 mol equiv.) was added dropwise, stirred for 2.5 min (no filterable solids), and concentrated to dryness. Yield: 23.8 g crude bindarit-carnosine conjugate.

Crude bindarit-carnosine conjugate was purified using flash chromatography (Biotage Isolera One). ~23.8 g crude bindarit-carnosine conjugate was loaded onto a 100 g sfar normal phase 20 μm silica column using 50 mL DCM: MeOH (75:25) via syringe. Pure bindarit-carnosine conjugate was eluted using a DCM: MeOH solvent system as follows: 6.3 CV (95:5); 10 CV (95:5 to 60:40). Yield: 1.55 g bindarit-carnosine conjugate (1.1% DCM and ~4.2% MeOH remaining (% w/w-NMR estimate).

### Hydrolysis Studies of Bindarit-Taurine Conjugate

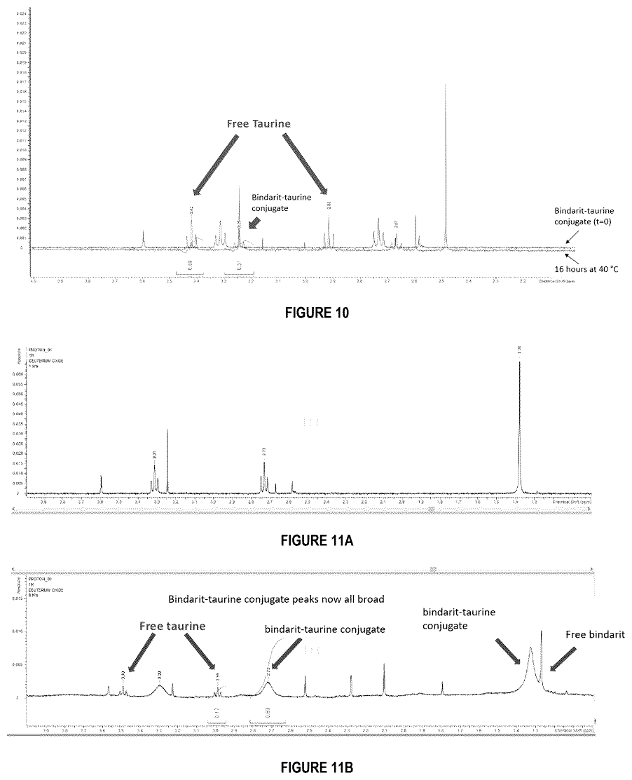

Acid hydrolysis: 4 mg bindarit-taurine conjugate was added to 4 mL 12M HCl. The reaction mixture was heated to 40° C. and stirred for 16 hours, followed by concentration of the reaction mixture. About 70% (mol %) of the bindarit-taurine conjugate was hydrolized as identified by NMR in D2O (FIG. 10).

Enzyme hydrolysis (esterase): 3 mg bindarit-taurine conjugate and 6 mg esterase enzyme were added to a vial. 3 mL of a 0.1M NaHCO₃(pH ~8) solution was added. The reaction mixture was heated to 40° C. and stirred for 18 hours, followed by concentration of the reaction mixture. About 17% (mol %) of the bindarit-taurine conjugate was hydrolized as identified by NMR in D2O (FIG. 11A) and demonstrating broadening of bindarit-taurine conjugate peaks (FIG. 11B).

### Hydrolysis Studies of Bindarit-Camosine Conjugate

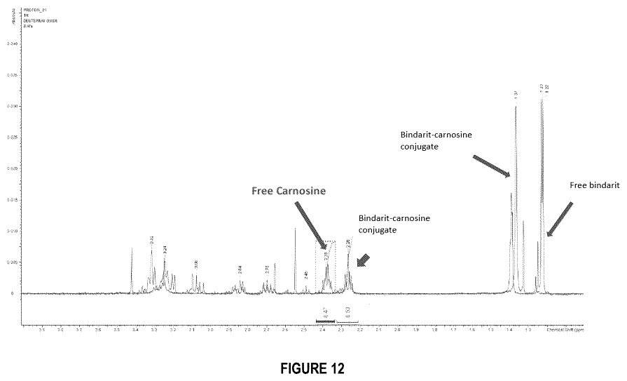

3 mg bindarit-carnosine conjugate was added to 3 mL 12M HCl. The reaction mixture was heated to 40° C. and stirred for 16 hours, followed by concentration of the reaction mixture. About 50% (mol %) of the bindarit-carnosine conjugate was hydrolized as identified by NMR in D2O (FIG. 12).

### Functional Assay Studies of Bindarit Conjugates

Enzyme hydrolysis (amidase): Bindarit-taurine conjugate and amidase enzyme (e.g., fatty acid amide hydrolase) is added to a vial. NaHCO₃(PH ~8) solution will be added. The reaction mixture is heated to 40° C. and stirred for 16 hours, followed by concentration of the reaction mixture.

In vitro assays are performed to test the functional results of the bindarit conjugates of the present disclosure (e.g., bindarit-resveratrol, bindarit-taurine, and bindarit-carnosine). Various cell types are used such as immortalized human retinal pigment epithelium (e.g., hTERT-RPE), or endothelial cells (e.g., microvasculature cells, such as retina cells). The cells are cultured in standard culture media and treated with different concentrations (e.g., about 2.5 mM or about 10 mM) of H2O2 or NalO3. Cell viability assays (e.g., alamarBlue Cell Viability Reagent) are used to assess the cells.

The controls that are used in the cell viability assays are cells only and cells treated with oxidizing agent in various concentrations (e.g., about 2.5 mM or about 10 mM of H2O2 or NalO3). Another set of controls that are used in the cell viability assays are cells treated with bindarit only, cells treated with resveratrol only, cells treated with taurine only, and cells treated with carnosine only. The experimental groups that are used in the cell viability assays are cells treated with bindarit-resveratrol, cells treated with bindarit-taurine, and cells treated with bindarit-carnosine.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

What is claimed is:

1. A compound of formula (I), or a pharmaceutically acceptable salt thereof: 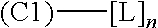
wherein in formula (I):
C1 is bindarit or an analogue thereof;
L is independently at each occurrence a linker covalently linked to C1, wherein each L is optionally further covalently linked to an active agent C2, and each occurrence of C2 may be the same or different; and
n is an integer selected from 1 to 4.

2. The compound of claim 1, wherein C1 comprises a group selected from:

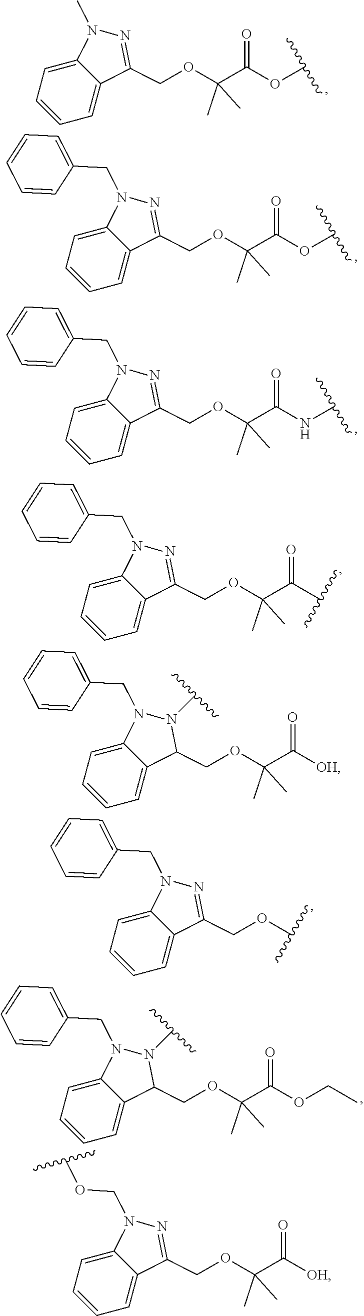

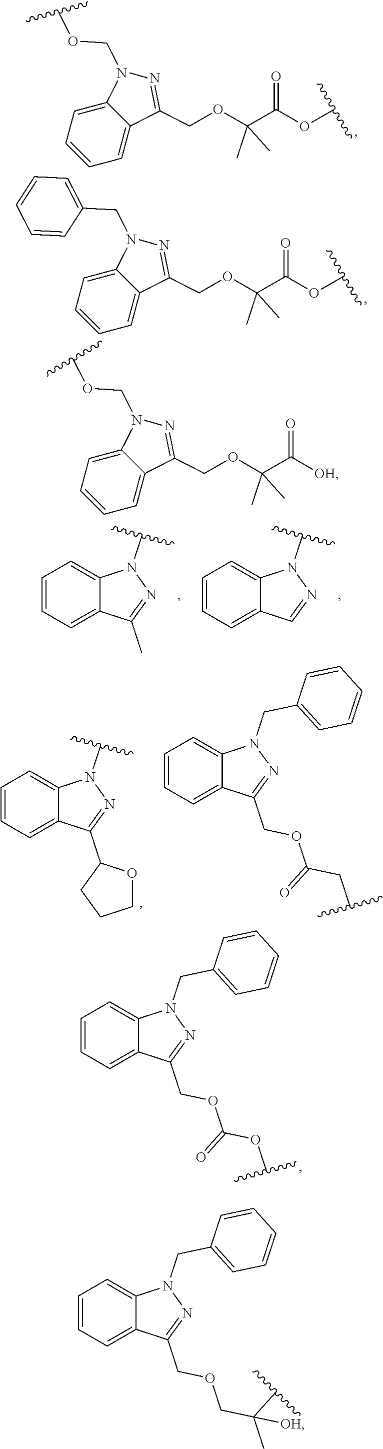

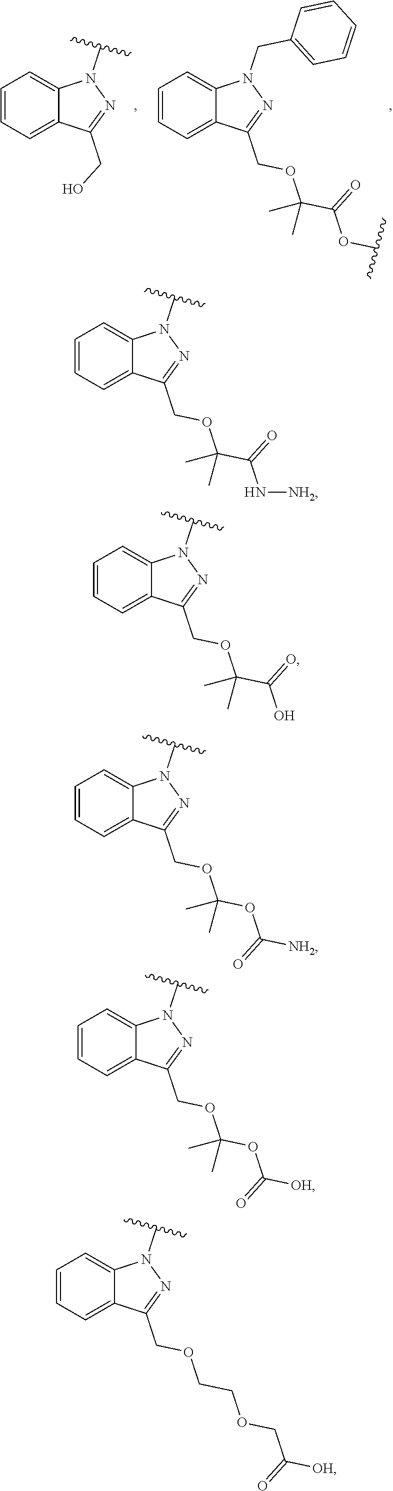

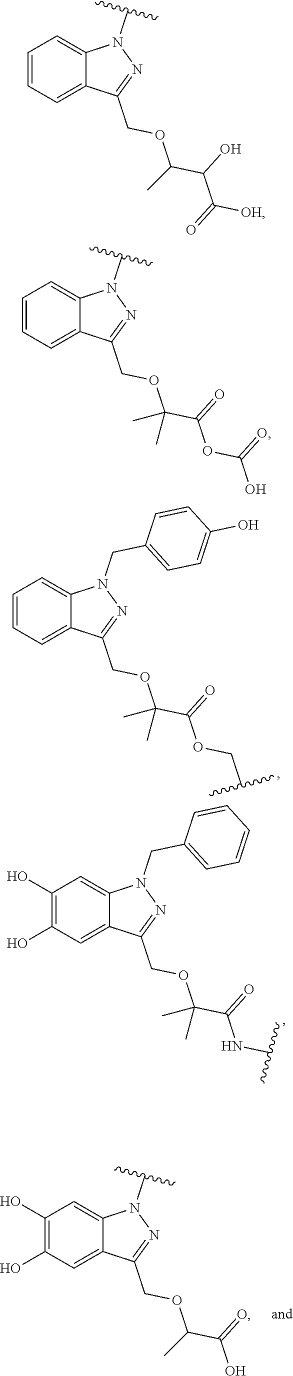

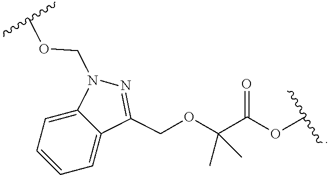 [SMILES: *OCn1nc(COC(C)(C)C(=O)O*)c2ccccc21]

3. The compound of claim 1 or 2, wherein the compound of formula (I) is a compound of formula (Ia), formula (Ib), formula (Ic), formula (Id), formula (Ie), formula (Ia1), formula (Ia2), or formula (Ia3), or a pharmaceutically acceptable salt thereof:

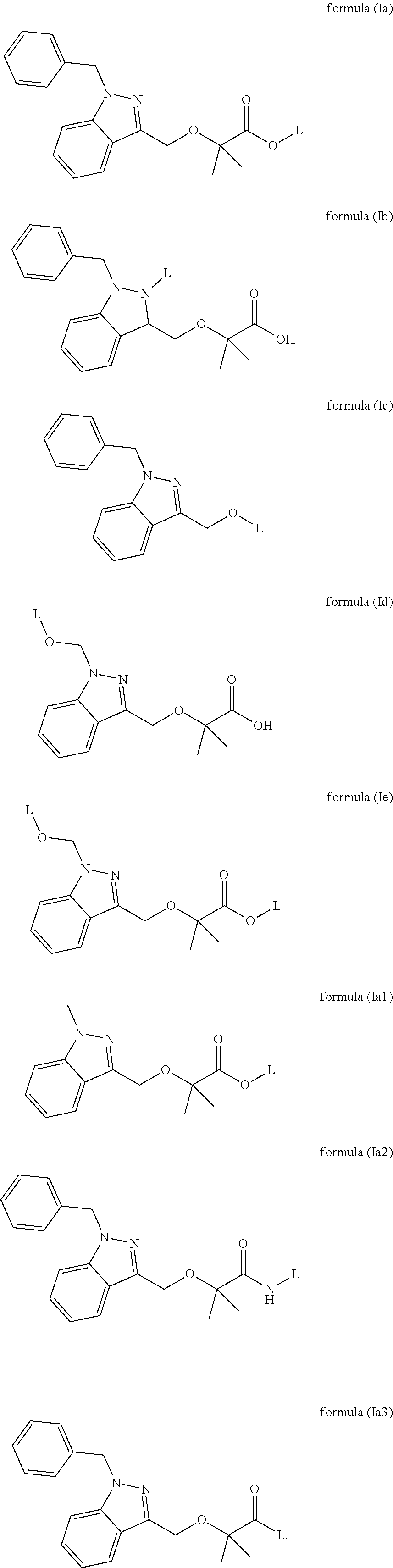

4. The compound of claim 1, wherein the compound of formula (I) is a compound of any one of formula (IIa), (Iic), (i2a), or (i2b): 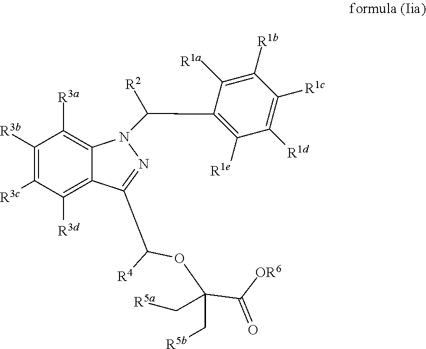
wherein in formula (Iia):
one to four of R^{1a}, R^{1b}, R^{1c}, R^{1d}, R^{1e}, R², R^{3a}, R^{3b}, R^{3c}, R^{3d}, R⁴, R^{5a}, R^{5b}, and R⁶ each independently represents -L or -L-C2 when L is further covalently linked to C2, and the remaining R^{1a}, R^{1b}, R^{1c}, R^{1d}, R^{1e}, R², R^{3a}, R^{3b}, R^{3c}, R^{3d}, R⁴, R^{5a}, R^{5b}, and R⁶ are independently selected at each occurrence from hydrogen, deuterium, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂;
L is independently at each occurrence a linker, wherein each Lis optionally further covalently linked to an active agent C2, and each occurrence of C2 may be the same or different; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; 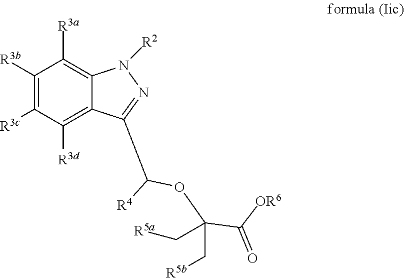
wherein in formula (Iic):
one to four of R², R^{3a}, R^{3b}, R^{3c}, R^{3d}, R⁴, R^{5a}, R^{5b}, and R⁶ each independently represents -L or -L-C2 when L is further covalently linked to C2, and the remaining R², R^{3a}, R^{3b}, R^{3c}, R^{3d}, R⁴, R^{5a}, R^{5b}, and R⁶ are independently selected at each occurrence from hydrogen, deuterium, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O), R^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a}) 2;
L is independently at each occurrence a linker, wherein each L is optionally further covalently linked to an active agent C2, and each occurrence of C2 may be the same or different; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; 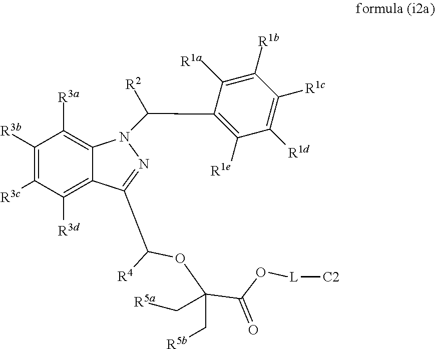
wherein in formula (i2a):
R^{1a}, R^{1b}, R^{1c}, R^{1d}, R^{1e}, R², R^{3a}, R^{3b}, R^{3c}, R^{3d}, R⁴, R^{5a}, and R^{5b} are independently selected at each occurrence from hydrogen, deuterium, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)OR^{a} (where t is 1 or 2), —S(O)ₜN(R^{a}) 2 (where t is 1 or 2), PO₃(R^{a})₂, -L, and -L-C2 when L is further covalently linked to C2;
L is independently at each occurrence a linker, wherein each L is optionally further covalently linked to an active agent C2;
C2 is an active agent, wherein each occurrence of C2 may be the same or different; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; 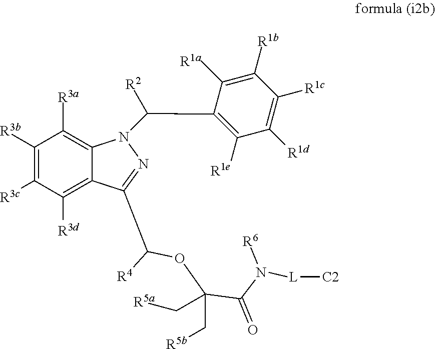
wherein in formula (i2a):
R^{1a}, R^{1b}, R^{1c}, R^{1d}, R^{1e}, R², R^{3a}, R^{3b}, R^{3c}, R^{3d}, R⁴, R^{5a}, R^{5b} and R⁶ are independently selected at each occurrence from hydrogen, deuterium, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), PO₃(R^{a})₂, -L, and -L-C2 when L is further covalently linked to C2;
L is independently at each occurrence a linker, wherein each L is optionally further covalently linked to an active agent C2;
C2 is an active agent, wherein each occurrence of C2 may be the same or different; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

5. The compound of claim 4, wherein at least one of (a)-(e) is satisfied:
(a) R^{1a}, R^{1b}, R^{1c}, R^{1d}, and R^{1e} are each hydrogen;
(b) R² is hydrogen;
(c) R^{3a}, R^{3b}, R^{3c}, and R^{3d} are each hydrogen;
(d) R⁴ is hydrogen; or
(e) R^{5a} and R^{5b} are each hydrogen.

6. The compound of claim 1, wherein C1 is bindarit ethyl ester or an analogue thereof, optionally wherein the bindarit ethyl ester is a compound of formula (IIb): 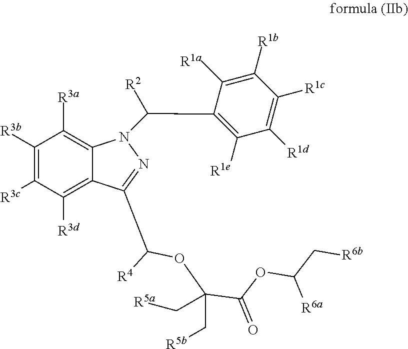
wherein in formula (IIb):
one to four of R^{1a}, R^{1b}, R^{1c}, R^{1d}, R^{1e}, R², R^{3a}, R^{3b}, R^{3c}, R^{3d}, R⁴, R^{5a}, R^{5b}, R^{6a}, and Rob each independently represents -L or -L-C2 when L is further covalently linked to C2, and the remaining R^{1a}, R^{1b}, R^{1c}, R^{1d}, R^{1e}, R², R^{3a}, R^{3b}, R^{3c}, R^{3d}, R⁴, R^{5a}, R^{5b}, R^{6a}, and R^{6b} are independently selected at each occurrence from hydrogen, deuterium, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a}) 2 (where t is 1 or 2), and PO₃(R^{a})₂;
L is independently at each occurrence a linker, wherein each Lis optionally further covalently linked to an active agent C2, and each occurrence of C2 may be the same or different; and R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

7. The compound of any one of claims 1-6, wherein each L independently comprises one or more of a bond, —(CH₂CH₂O)—, —C(O), —(CH₂)—, —O—, —C(O)O—, —OC(O)—, —C(O)NR^{a}—, —NR^{a}C(O)—, —S—, —S—S—, —SCH₂CH₂S—, —NR^{a}—, —S(O)—, —S(O)₂—, —CR^{a}₂—, —C(O)S—, —SC(O)—, —C(O)NR^{a}SO₂—, —SO₂NR^{a}C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

8. The compound of any one of claims 1-7, wherein each L independently comprises one or more of a PEG-based linker, polyester linker, polypropylene, polyolefin, polyolefin, polyurethane, Alhydrogel, vitamin K, caffeine, menadione, benzoic acid, cholesterol, PVP, β-cyclodextrin, 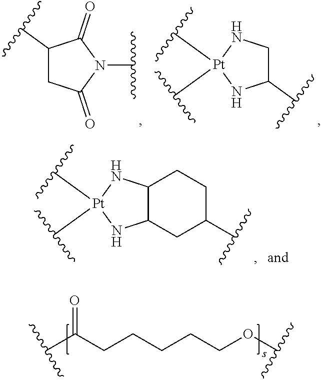
wherein s is 1 to 5, optionally wherein the polyester is selected from: polylactic acids, poly-L-lactic acid (PLLA), and a random copolymer of L-lactic acid and D-lactic acid, and derivatives thereof, polycaprolactone, polyhydroxybutyric acid, polyhydroxyvaleric acid, polyethylene succinate, polybutylene succinate, polybutylene adipate, polymalic acid, polyglycolic acid, polysuccinate, polyoxalate, polybutylene diglycolate, and polydioxanone.

9. The compound of claim 8, wherein the PEG-based linker comprises one or more of an amine reactive PEG-based linker (e.g. Bis-PEG-acid, Bis-PEG-NHS, Boc-PEG, Fmoc-PEG, PEG Acid, PEG Aldehyde, PEG NHS ester, PEG Phosphonate, PEG PFP ester, PEG Tosylate), a biotinylated PEG-based linker (e.g. PEG-biotin), a branched PEG-based linker; a reactive carbonyl PEG-based linker (e.g. aminooxy-PEG), a carboxyl and/or active ester reactive PEG-based linker (e.g. amine PEG), a click-reagent PEG-based linker (e.g. alkyne PEG, azide-PEG); a copper-free click chemistry PEG-based linker (e.g. DBCO-PEG, BCN-PEG); a Cu-catalyzed click chemistry PEG-based linker (e.g. propargyl-PEG); a fluorescent PEG labeling and homobifunctional PEG-based linker (e.g. bis-PEG-acid, bis-PEG-NHS, bis-PEG-PFP, bis-propargyl-PEG, amine-PEG-amine, azido-PEG-azide, bromo-PEG-bromide), a lipid PEG, a metal surface binding and thiol reactive PEG-based linker (e.g. Thiol PEG, Bromo-PEG, Mal PEG), PEG-PAsp, PEG-PGlu, PEG-b-PMPMC, PEG-PGLA, 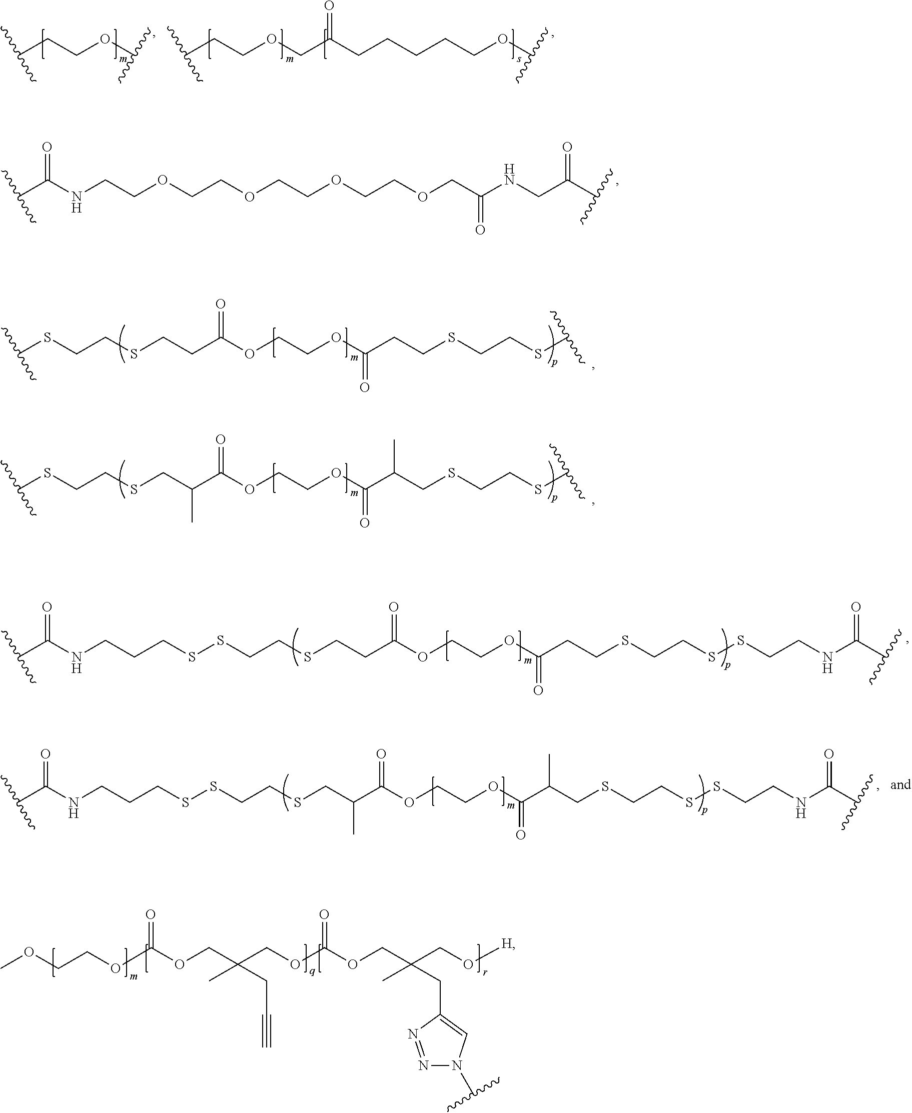
wherein:
m is an integer selected from 1 to 5;
p is an integer selected from 1 to 3;
q is an integer selected from 1 to 5;
r is an integer selected from 1 to 5; and
s is an integer selected from 1 to 5.

10. The compound of one of claim 1, 2, or 4-9, wherein one or more L is further covalently linked to C2.

11. The compound of claim 10, wherein C2 comprises one or more of an anti-vascular endothelial growth factor (VEGF) agent, a modulator of the complement cascade, a non-steroidal anti-inflammatory drug (NSAID), an angiotensin-converting enzyme (ACE) inhibitor, a peroxisome proliferator-activated receptor (PPAR) modulator, a renin inhibitor, a corticosteroid, an antibody, a soluble receptor, a biological molecule, a nuclear receptor protein (e.g. LXR), a Liver X receptor (LXR) agonist, a steroid (e.g. fluocinolone acetonide, dexamethasone), a fatty acid mimic (FAM) molecule, and an agent that modulates autophagy.

12. The compound of claim 11, wherein the PPAR modulator is selected from a PPAR-α modulator, a PPAR-β/δ modulator, and a PPAR-γ modulator.

13. The compound of claim 11 or 12, wherein the PPAR modulator is selected from a PPAR agonist, a PPAR antagonist, a PPAR partial agonist, and a PPAR inverse agonist.

14. The compound of claim 11, wherein the non-steroidal anti-inflammatory drug (NSAID) is selected from indomethacin (Indocin), ibuprofen, diclofenac (Voltaren), acemetacin, diclofenac, naproxen, ketorolac, carprofen, etodolac, fentiazac, flurbiprofen, ketoprofen, oxaprozin, pranoprofen, sulindac, suxibuzone, celecoxib (Celebrex®), fenoprofin (Nalfon), ketorolac tromethamine, tolmetin, diflunisal, meclofenamate sodium, mefenamic acid, etoricoxib, meloxicam, nabumetone, and piroxicam, optionally wherein the NSAID is indomethacin (Indocin), optionally wherein the NSAID is ibuprofen.

15. The compound of claim 11 or 14, wherein C1 comprises bindarit or an analogue thereof and C2 is or comprises an NSAID.

16. The compound of claim 15, wherein C1, C2, and/or L is deuterated.

17. The compound of claim 16, wherein the C1, C2, and/or L is deuterated at one or more sites different from the about 3, about 4, about 5, about 6, about 7, or about 8 sites of C1, C2, and/or L that binds and/or interacts with the pocket of the beta barrel domain.

18. The compound of claim 10 or 11, wherein C2 comprises one or more of bindarit or an analogue thereof, an anti-angiogenic agent, optionally a biological anti-angiogenic agent, (e.g. angiopoietin and placental growth factor), an anti-VEGF agent, optionally a low molecular weight anti-VEGF agent, a nutraceutical, optionally resveratrol, one or more peptides, optionally taurine, carnosine or aspartame, or one or more proteins, optionally an anti-VEGF antibody or a soluble receptor.

19. The compound of claim 18, wherein the anti-angiogenic agent is selected from Axitinib (Inlyta), Bevacizumab (Avastin), Cabozantinib (Cometriq), Everolimus (Afinitor, Zortress), Lenalidomide (Revlimid), Pazopanib (Votrient), Ramucirumab (Cyramza), Regorafenib (Stivarga), Sorafenib (Nexavar), Sunitinib (Sutent), Thalidomide (Synovir, Thalomid), Vandetanib (Caprelsa), Ziv-aflibercept (Zaltrap), Aflibercept (Eylea), Ranibizumab (Lucentis), Brolucizumab, Abicipar (a DARPin), KSI-301 Antibody Biopolymer Conjugate (Kodiak sciences (e.g. for extended release in ophthalmology), OPT-302 (Opthea) (VEGF-C/D trap molecule), Faricimab (anti-VEGF & anti-angiopoietin-2), DE-122, and anti-endoglin (Tracon & Santen) (e.g. for anti-angiogenesis in ophthalmology).

20. The compound of claim 18, wherein the nutraceutical agent is selected from nutraceutical agents include, resveratrol, taurine, carnosine, quercetin, curcumin, glycyrrhizin, naringin, folate, glutathione, n-acetylcysteine, alpha lipoic acid, quercetin, zinc, vitamin a, vitamin c, vitamin d, vitamin k2, vitamin b, vitamin b6, vitamin b9, vitamin b12, vitamin e, magnesium, bromelain, cannabidiolic acid, cannabigerolic acid, cannabidiol, cannabigerol, cannabinol, nigeIIa sativa/thymoquinone, selenium, curcumin, piperine, astaxanthin, and sulforaphane.

21. The compound of claim 18, wherein the peptide is selected from a monopeptide (e.g. taurine or carnosine) and a dipeptide (e.g. aspartame and glutamine-taurine).

22. The compound of claim 18, wherein the protein is selected from an anti-VEGF antibody (e.g. Ranibizumab) or a soluble receptor (e.g. solVEGFR1 [sol flt-1] or solVEGFR2).

23. The compound of claim 18, wherein the low molecular weight anti-VEGF agent is selected from thalidomide (Synovir, Thalomide), an mTOR inhibitor (e.g. temsirolimus, everolimus (Afinitor, Zortress), tacrolimus or rapalogues (e.g. rapamycin), or corticosteroids (dexamethasone, Fluocinolone acetonide, triamcinolone), aflibercept, and a tyrosine kinase inhibitor.

24. The compound of claim 23, wherein the tyrosine kinase inhibitor is selected from Vatalanib (PTK787), lapatinib, Sunitinib (Sutent), Sorafenib (Nexavar), Ramucirumab (Cyramza), Nintedanib, Lenvatinib, Anlotinib, Fruquintinib, Apatinib, Pazopanib (Votrient), Axitinib (Inlyta), Cabozantinib (Cometriq), Vandetanib (Caprelsa), and Regorafenib (Stivarga).

25. The compound of any one of claims 1-24, wherein C2 comprises a polyphenol, optionally wherein the polyphenol is selected from catechin, resveratrol, hesperidin, chlorogenic acid, flavonols, isoflavones, tannins, quercetin, and anthocyanins.

26. The compound of claim 25, wherein C2 comprises resveratrol.

27. The compound of claim 11, wherein the biological molecule is an antibody, an antibody fragment, a soluble receptor (e.g. solVEGFR1 [sol flt-1] or solVEGFR2), a DARPin, a microRNA (miRNA), Bevacizumab (Avastin), Ziv-aflibercept (Zaltrap), Aflibercept (Eylea), Ranibizumab (Lucentis), Brolucizumab (Beovu), Abicipar (AbbVie, a DARPin), KSI-301 Antibody Biopolymer Conjugate (Kodiak sciences) (e.g. for extended release in ophthalmology), OPT-302 (Opthea) (VEGF-C/D trap molecule), Faricimab (anti-VEGF & anti-angiopoietin-2), and DE-122, and anti-endoglin (Tracon & Santen) (e.g. for anti-angiogenesis in ophthalmology).

28. The compound of any one of claims 18-27, wherein bindarit or the analogue thereof comprises a group selected from:

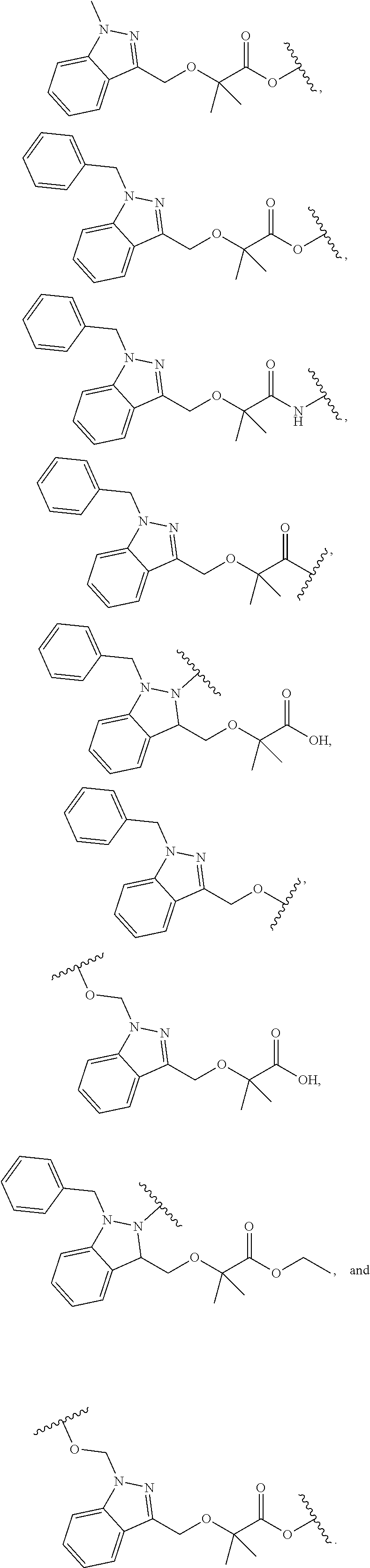

29. The compound of any one of claims 1, 2, and 4-28 wherein the compound of formula (I) is a compound of formula (If), formula (Ig), formula (Ih), formula (Ii), formula (Ij), formula (Ik), formula (Im), formula (In), or a pharmaceutically acceptable salt thereof:

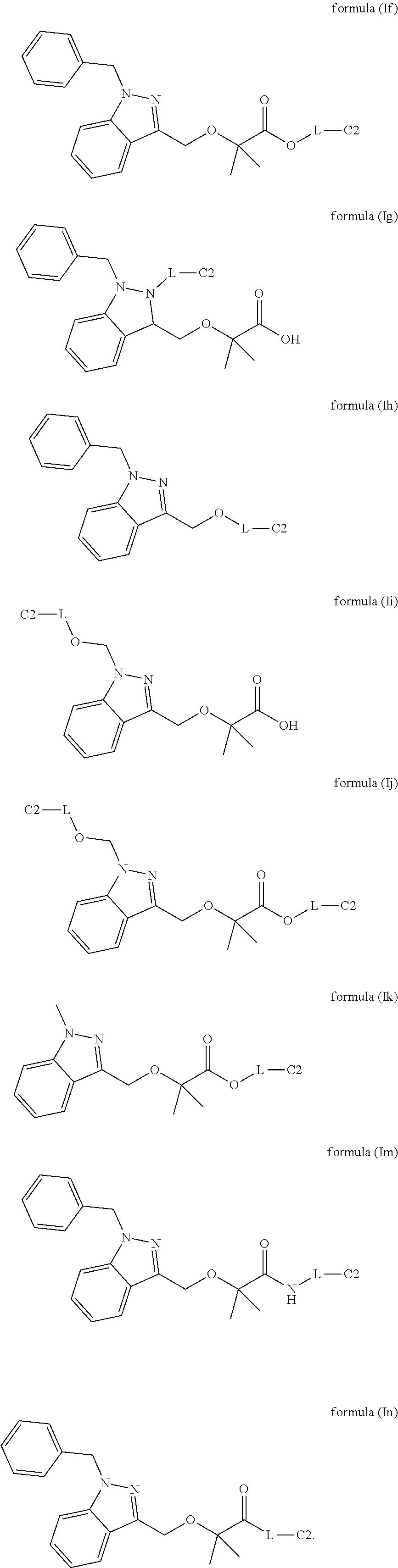

30. The compound of claim 1, wherein the compound is a compound of formula (IV), formula (V), formula (VI), or formula (VII), or a pharmaceutically acceptable salt thereof:

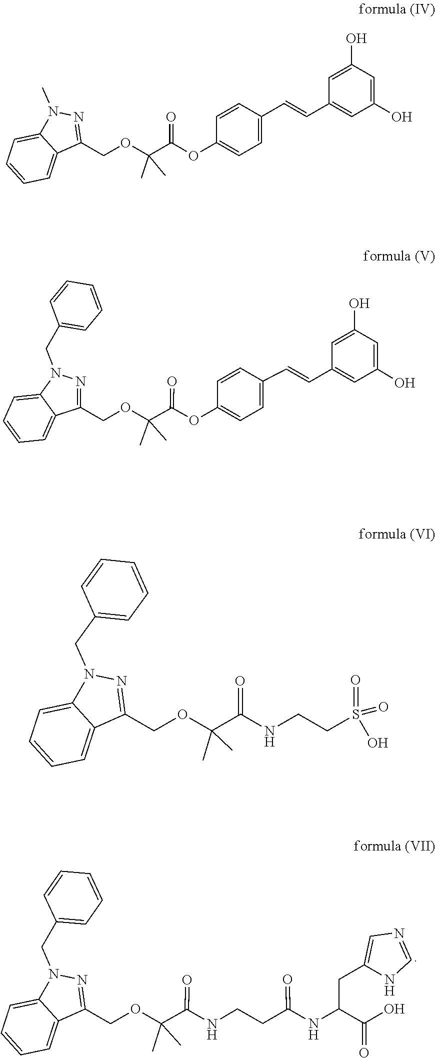 [SMILES: Cn1nc(COC(C)(C)C(=O)Oc2ccc(/C=C/c3cc(O)cc(O)c3)cc2)c2ccccc21 ; CC(C)(OCc1nn(Cc2ccccc2)c2ccccc12)C(=O)Oc1ccc(/C=C/c2cc(O)cc(O)c2)cc1 ; CC(C)(OCc1nn(Cc2ccccc2)c2ccccc12)C(=O)NCCS(=O)(=O)O ; CC(C)(OCc1nn(Cc2ccccc2)c2ccccc12)C(=O)NCCC(=O)NC(Cc1cnc[nH]1)C(=O)O]

31. The compound of claim 1, wherein the compound of formula (VII) is a compound of formula (VIla) or formula (VIIb), optionally a compound of formula (VIIa), or a pharmaceutically acceptable salt thereof:

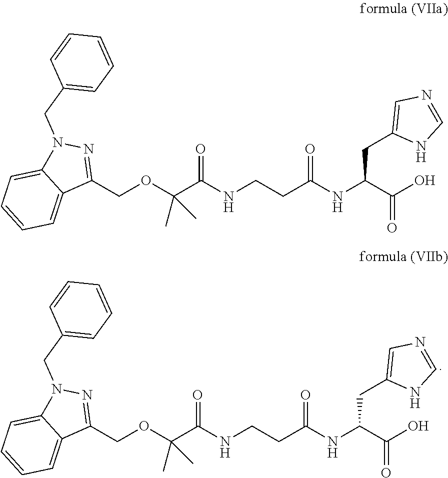 [SMILES: CC(C)(OCc1nn(Cc2ccccc2)c2ccccc12)C(=O)NCCC(=O)N[C@@H](Cc1cnc[nH]1)C(=O)O ; CC(C)(OCc1nn(Cc2ccccc2)c2ccccc12)C(=O)NCCC(=O)N[C@H](Cc1cnc[nH]1)C(=O)O]

32. The compound of any one of claims 1-26, wherein the compound of formula (I) is a macrocycle.

33. The compound of claim 32, wherein the compound of formula (I) is a compound of formula (IIIa):

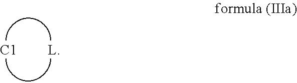

34. The compound of claim 33, wherein the compound of formula (I) is a compound of formula (Iic):

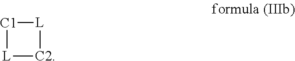

35. The compound of any one of claim 1, 2, 4-32, or 34 wherein the compound of formula (I) is a homodimer.

36. The compound of any one of claim 1, 2, 4-32, or 34 wherein the compound of formula (I) is a heterodimer.

37. The compound of claim 1, wherein the compound of formula (I) is of any one of formulas 1001 to 1005, or a pharmaceutically acceptable salt thereof:

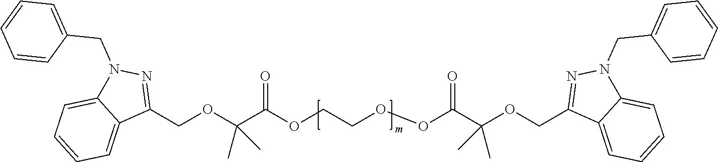

| Compound no. | m |
|---|---|
| 1001 | 1 |
| 1002 | 2 |
| 1003 | 3 |
| 1004 | 4 |
| 1005 | 5 |

38. The compound of claim 1, wherein:
C1 is selected from bindarit or an analogue thereof and bindarit ethyl ester or an analogue thereof;
L comprises one or more of a bond, —(CH₂CH₂O)—, —C(O), —(CH₂)—, —O—, —C(O)O—, —OC(O)—, —C(O)NR^{a}—, —NR^{a}C(O)—, —S—, —S—S—, —SCH₂CH₂S—, —NR^{a}—, —S(O)—, —S(O)₂—, —CR^{a}₂—, —C(O)S—, —SC(O)—, —C(O)NR^{a}SO₂—, —SO₂NR^{a} C(O)—, —OC(O)—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl:
L is further covalently linked to C2;
C2 is selected from PTK787, aflibercept, Brolucizumab, Triamcinolone, Dexamethasone, OPT-302, and DE-122; and
n is 1.

39. The compound of claim 38, wherein L independently comprises one or more of a PEG-based linker, polyester, polypropylene, polyolefin, polyurethane, Alhydrogel, vitamin K, caffeine, menadione, benzoic acid, cholesterol, PVP, β-cyclodextrin, 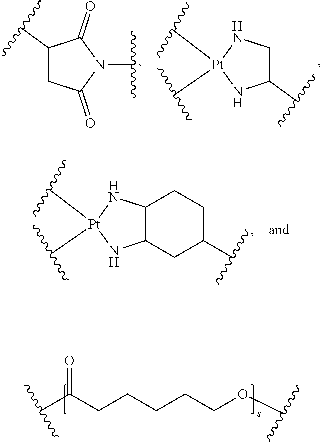
wherein s is 1 to 5.

40. The compound of claim 39, wherein the PEG-based linker comprises one or more of an amine reactive PEG-based linker (e.g. Bis-PEG-acid, Bis-PEG-NHS, Boc-PEG, Fmoc-PEG, PEG Acid, PEG Aldehyde, PEG NHS ester, PEG Phosphonate, PEG PFP ester, PEG Tosylate), a biotinylated PEG-based linker (e.g. PEG-biotin), a branched PEG-based linker; a reactive carbonyl PEG-based linker (e.g. aminooxy-PEG), a carboxyl and/or active ester reactive PEG-based linker (e.g. amine PEG), a click-reagent PEG-based linker (e.g. alkyne PEG, azide-PEG); a copper-free click chemistry PEG-based linker (e.g. DBCO-PEG, BCN-PEG); a Cu-catalyzed click chemistry PEG-based linker (e.g. propargyl-PEG); a fluorescent PEG labeling and homobifunctional PEG-based linker (e.g. bis-PEG-acid, bis-PEG-NHS, bis-PEG-PFP, bis-propargyl-PEG, amine-PEG-amine, azido-PEG-azide, bromo-PEG-bromide), a lipid PEG, a metal surface binding and thiol reactive PEG-based linker (e.g. Thiol PEG, Bromo-PEG, Mal PEG), PEG-PAsp, PEG-PGlu, PEG-b-PMPMC, PEG-PGLA, 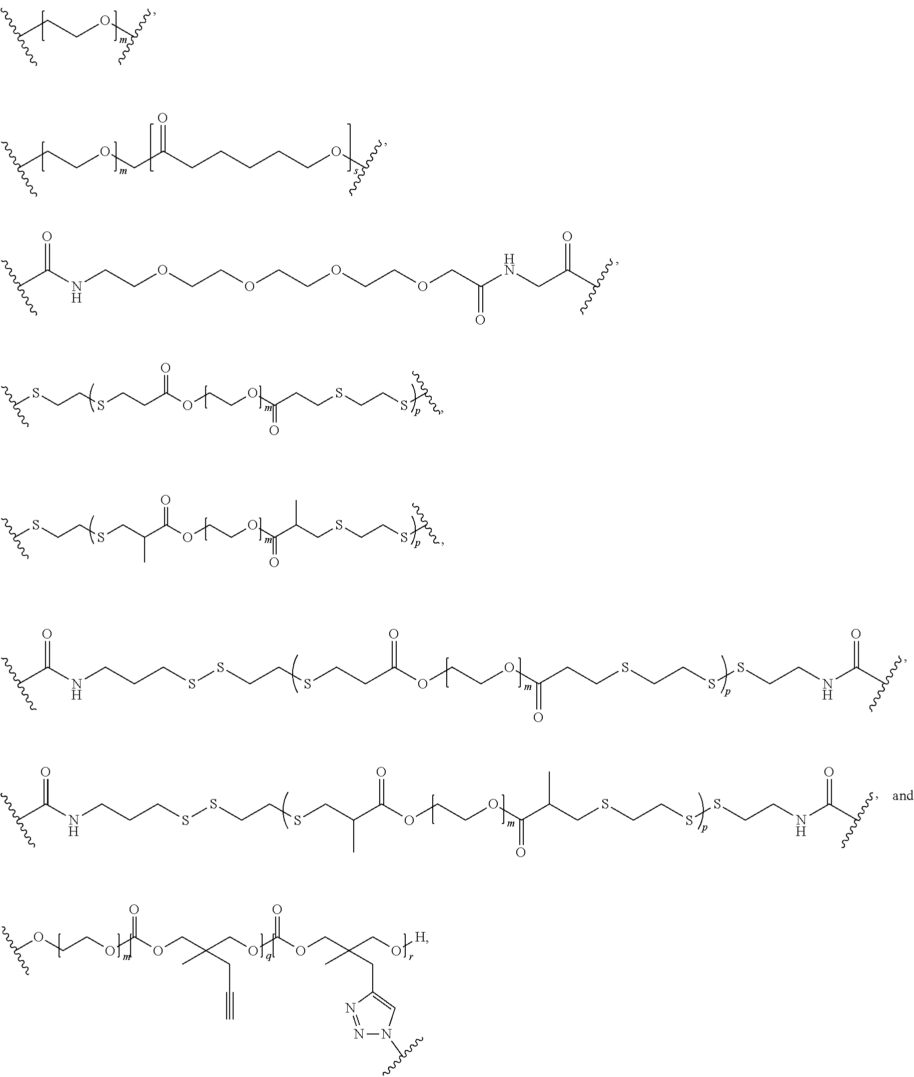
wherein:
m is an integer selected from 1 to 5;
p is an integer selected from 1 to 3;
q is an integer selected from 1 to 5;
r is an integer selected from 1 to 5; and
s is an integer selected from 1 to 5.

41. A pharmaceutical composition comprising one or more compounds of any one of claims 1 to 40, or a pharmaceutically acceptable salt, thereof, and a pharmaceutically acceptable carrier.

42. The pharmaceutical composition of claim 41, wherein the pharmaceutical composition comprises two or more compounds, wherein at least one compound is a homodimer and at least one compound is a heterodimer.

43. The pharmaceutical composition of claim 42, wherein the molar ratio of the homodimer to the heterodimer is selected from between about 1:99 to about 99:1.

44. The pharmaceutical composition of any one of claims 41-43, wherein the pharmaceutical composition is formulated for extended release.

45. The pharmaceutical composition of any one of claims 41-44, wherein the pharmaceutical composition is formulated for ocular delivery.

46. A method for treating or preventing an ocular disease or disorder, comprising administering to a subject in need thereof a pharmaceutical composition comprising an effective amount of a compound of any one of claims 1-40 and a pharmaceutically acceptable carrier or excipient.

47. The method of claim 46, wherein the ocular disease or disorder is one or more of dry age-related macular degeneration (AMD), reticular drusenoid disease (RPD), white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), diffuse subretinal fibrosis (DSF)), LORDs, and central serous retinopathy (CSR).

48. The method of claim 46 or 47, wherein the ocular disease or disorder is one or more of Vogt-Kayanagi-Harada disease (VKH); Sarcoid uveitis; Ocular histoplasmosis and/or Presumed Ocular Histoplasmosis Syndrome; Autoimmune uveitis; Uveitis associated with systemic diseases, e.g. lupus, Crohn's disease, rheumatoid arthritis, and other diseases of known immune origin; Posterior uveitis (including that which may not yet be diagnosed); Anterior uveitis (e.g. iritis); Bechet's disease; Polyarteritis nodosa; Wegener granulomatosis, and optic neuritis, optionally wherein the optic neuritis is primary or secondary to systemic (i.e. non-ocular) disease.

49. The method of any one of claims 46-48, wherein the method further comprises reducing the subject's expression of one or more of CD64, IDO, SOCS1, CXCL10 Marco, Nos2, 1112b, Ptgs2 (Cox2), MCP-1 (CCL2), MCP-2 (CCL8), MCP-3 (CCL7), 1123a (H23p19), Idol, Adipoq, Ccl20, IL17 (subtype a)Ccl5, CD163, Cx3Cr1, Faslg, Gfap, Csf2, Icaml, Ifng, 1110, 1112b, 1113, 1117, 1118, 1M b, 1122, 114, 116, Klf4, Mrd, Myd88, Nlrp3, Pparv, Tgfb1, Tlr4, Tnf, Vcaml, Ccl2, Ccl5, Ccl7, Ccr2, Socsl, Socs3, Statl, Stat3, and Stat6.

50. The method of any one of claims 46-49, wherein the subject is not undergoing treatment with and/or is unresponsive and/or is substantially unresponsive and/or is partially responsive to one or more of an anti-factor D antibody (e.g. lampalizumab (Genentech)), an anti-β-amyloid (anti-Aβ) antibody (e.g. GSK933776 (GSK)), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), Zimura (Iveric), an anti-C3 complement antibody (e.g. APL-2 (Apellis) or related compounds thereof, NGM621 (NGM Biopharmaceutical)), doxycycline (ORACEA), emixustat hydrochloride, rapalogues (e.g. sirolimus (RAPAMUNE), metformin or analogues thereof, glatiramer acetate (COPAXONE), and an NSAID (e.g. ibuprofen, indomethacin).

51. The method of any one of claims 46-50, wherein the subject has evidence of intermediate AMD as confirmed by the presence of at least 1 large druse greater than about 125 μm in diameter and/or pigment.

52. The method of any one of claims 46-51, wherein the subject has early stage dry macular degeneration as evidenced by small drusen and/or medium-sized drusen and/or large drusen and/or reticular pseudodrusen, and/or late AMD and/or dry AMD and/or atrophic AMD as evidence by nascent, incomplete, emerging or evolving geographic atrophy and/or complete geographic atrophy.

53. The method of any one of claims 46-51, wherein the subject has early, intermediate, or late stage dry macular degeneration as evidenced by a large number of medium-sized drusen, pigment, and/or atrophy.

54. The method of any one of claims 46-51, wherein the subject has early AMD with drusen, intermediate AMD with drusen and/or pigment, or late dry macular degeneration as evidenced first by nascent, incomplete, emerging, evolving geographic atrophy, and complete geographic atrophy that continues to expand over time.

55. The method of any one of claims 46-51, wherein the subject has reticular pseudodrusen, or early or late stage dry AMD as evidenced by subretinal drusenoid deposits.

56. The method of any one of claims 46-55, wherein the subject has a variant of AMD.

57. The method of claim 56, wherein the variant of AMD is selected from polypoidal choroidal vasculopathy (PCV) and retinal angiomatous proliferation (RAP).

58. The method of any one of claims 46-57, wherein the subject has no evidence of prior or active choroidal neovascularization (CNV).

59. The method of any one of claims 46-57, wherein the subject has evidence of prior or active choroidal neovascularization (CNV).

60. The method of any one of claims 46-59, wherein the subject has one or more disease features suggesting the subject is at risk for CNV such as sub- or intra-retinal fluid, sub-RPE fluid, non-exudative CNV, and a double-layer sign.

61. The method of any one of claims 46-60, wherein the subject has one or more well-demarcated GA lesions of a total area of about 0.2 to 2.0 to about 20 mm² using en face imaging in one or more eye, and/or a diameter of below about 200 μm, between about 200 and about 250 μm, or greater than about 250 μm, optionally measured using cross-sectional OCT.

62. The method of any one of claims 46-61, wherein the subject has a best-corrected visual acuity score of greater than about 35 letters or a Snellen VA equivalent of about 20/400 or better.

63. The method of any one of claims 46-62, wherein the subject has a GA lesion of less than one disc area up to more than 10 disc areas, or less than about 200 μm diameter up to more than about 500 μm.

64. The method of claim 46, wherein the ocular disease or disorder is a diabetic eye disease.

65. The method of claim 64, wherein the diabetic eye disease is diabetic retinopathy or diabetic macular edema (DME).

66. The method of claim 46, wherein the ocular disease or disorder is a cataract, glaucoma, or optic neuropathy.

67. The method of any one of claims 64-66, wherein the subject has type 1 or type 2 diabetes.

68. The method of claim 67, wherein the subject has a blood glucose level that exceeds normal levels.

69. The method of any one of claims 64-68, wherein the method further comprises reducing NFkb translocation to the nucleus in eye cells of the subject.

70. The method of any one of claims 64-69, wherein the method further comprises reducing translocation of, or activity of, nuclear receptors, to or within the nucleus in the eye cells of the subject, wherein reducing translocation or activity results in the alteration of transcriptional activity.

71. The method of any one of claims 64-70, wherein the method further comprises normalizing cellular stress in cells under hypoxic, oxidative, superoxide, nitrosyl or glycemic stress.

72. The method of any one of claims 64-71, wherein the method further comprises administering one or more additional therapeutic agents.

73. The method of claim 72, wherein the one or more additional therapeutic agents is selected from an anti-factor D antibody (e.g. lampalizumab (Genentech)), an anti-β-amyloid (anti-Aβ) antibody (e.g. GSK933776 (GSK)), a corticosteroid (e.g. fluocinolone acetonide), MC-1101 (MacuCLEAR), a CD34+ stem cell therapy, an anti-VEGF antibody (e.g. Ranibizumab), brimonidine (Alphagan), an anti-C5 complement antibody (e.g. LFG316 (Novartis), doxycycline (ORACEA), emixustat hydrochloride, sirolimus (RAPAMUNE), glatiramer acetate (COPAXONE), and an NSAID (e.g. ibuprofen, indomethacin).

74. The method of claim 72, wherein the one or more additional therapeutic agents is selected from an anti-VEGF agent, an ACE inhibitor, a PPAR-gamma agonist or partial agonist, a renin inhibitor, a steroid, an agent that modulates nuclear receptor activity or gene transcription, a rapalogue, metformin or analogues thereof, an agent that modulates autophagy, a semapimod, a MIF inhibitor, a CCR2 inhibitor, CKR-2B, a 2-thioimidazole, CAS 445479-97-0, CCX140, clodronate, a clodonate-liposome preparation and gadolinium chloride.

75. The method of claim 72, wherein the one or more additional therapeutic agents is one or more of an anti-vascular endothelial growth factor (VEGF) agent, a modulator of the complement cascade, an angiotensin-converting enzyme (ACE) inhibitor, a peroxisome proliferator-activated receptor (PPAR)-gamma agonist, a renin inhibitor, a corticosteroid, and an agent that modulates autophagy.

76. The method of claim 72, wherein the one or more additional therapeutic agents is a nutraceutical agent, optionally wherein the nutraceutical agent is selected from one or more of resveratrol, taurine, carnosine, quercetin, curcumin, glycyrrhizin, naringin, vitamin A, vitamin B, vitamin C, vitamin E, folate, and vitamin D.

77. The method of claim 72, wherein the one or more additional therapeutic agents is a fatty acid agent, optionally wherein the fatty acid agent is selected from one or more of polyunsaturated fatty acid (PUFA), monounsaturated fatty acid (MUFA), or a saturated fatty acid (SFA).

78. A compound of formula of formula (V), or a pharmaceutically acceptable salt thereof:

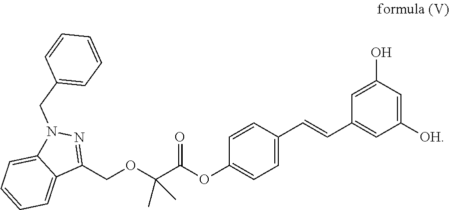 [SMILES: CC(C)(OCc1nn(Cc2ccccc2)c2ccccc12)C(=O)Oc1ccc(/C=C/c2cc(O)cc(O)c2)cc1]

79. A compound of formula (VI), or a pharmaceutically acceptable salt thereof:

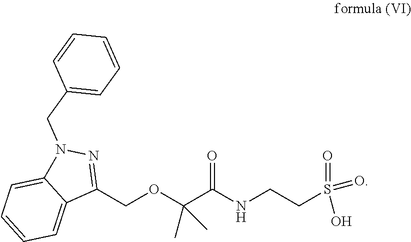 [SMILES: CC(C)(OCc1nn(Cc2ccccc2)c2ccccc12)C(=O)NCCS(=O)(=O)O]

80. A compound of formula (VIIa), or a pharmaceutically acceptable salt thereof: 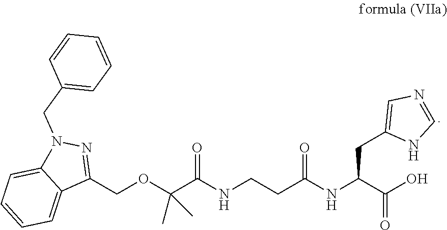 [SMILES: CC(C)(OCc1nn(Cc2ccccc2)c2ccccc12)C(=O)NCCC(=O)N[C@@H](Cc1cnc[nH]1)C(=O)O]
